(19) United States
(12) Patent Application Publication
(10) Pub. No.: US 2022/0218707 A1
(43) Pub. Date: Jul. 14, 2022

(54) USE OF COMPOSITION OF IMATINIB OR DERIVATIVE THEREOF IN PREPARATION OF DRUG FOR PREVENTING, TREATING, AND CONTROLLING ADDICTION RELAPSE

(71) Applicant: Wuhan University, Wuhan (CN)

(72) Inventors: Yanqin LI, Wuhan (CN); Shimin ZHU, Wuhan (CN); Jiawei RUAN, Wuhan (CN); Xinyu ZHANG, Wuhan (CN); Mingzhu CHEN, Wuhan (CN)

(21) Appl. No.: 17/709,358

(22) Filed: Mar. 30, 2022

Related U.S. Application Data
(63) Continuation-in-part of application No. PCT/CN2020/119253, filed on Sep. 30, 2020.

(30) Foreign Application Priority Data
Sep. 30, 2019 (CN) 201910939100.3
Sep. 30, 2019 (CN) 201910939113.0
Sep. 30, 2019 (CN) 201910939707.1

(51) Int. Cl. A61K 31/506; A61P 25/36; A61P 25/32

(52) U.S. Cl. CPC A61K 31/506; A61P 25/34; A61P 25/32; A61P 25/36

(57) ABSTRACT

A method for prevention and treatment of addictions and control of relapse comprising administering a patient in need thereof a composition comprising imatinib or a derivative thereof, and an addictive substance or an analgesic agent; wherein the addictive substance comprises: (1) narcotic drugs, (2) psychotropic drugs, (3) ethanol, tobacco, volatile organic solvents, and other substances leading to addictions in organisms, and analogues thereof; and (4) nicotine or an analogue thereof.

### CROSS-REFERENCE TO RELAYED APPLICATIONS

This application is a continuation-in-part of International Patent Application No. PCT/CN2020/119253 with an international filing date of Sep. 30, 2020, designating the United States, now pending, and further claims foreign priority benefits to Chinese Patent Application No. 201910939100.3 filed Sep. 30, 2019, to Chinese Patent Application No. 201910939113.0 filed Sep. 30, 2019, and to Chinese Patent Application No. 201910939707.1 filed Sep. 30, 2019. The contents of all of the aforementioned applications, including any intervening amendments thereto, are incorporated herein by reference.

### BACKGROUND

The disclosure relates to the technical field of medicine, and particularly to use of imatinib or a derivative thereof, in combination with an addictive substance and an addictive behavior, in the preparation of a compound or combined preparation for preventing and treating addiction and controlling relapse.

The drug problem is an urgent global public health problem having immeasurable impacts on society and patients. Also, major diseases and casualties caused by substance dependence, such as tobacco and alcohol addiction emerge endlessly. The addiction to neuropsychiatric drugs and analgesic drugs limits their use and brings irreversible adverse consequences to society and patients. Internet addiction, gambling, game addiction and other behavioral addictions bring more adverse consequences to people's lives. Drug addiction can lead to serious consequences; however, the trend of drug abuse is still increasing, and the underlying main cause is “addiction”. Once addiction, any substance or behavior is difficult to quit. So far, no solution to this problem is available in the world, and there is currently a lack of effective drugs.

The disclosure relates to the development of new therapeutic agents against addiction. The disclosure adopts a new concept, and finds that imatinib mesylate, a specific and effective drug for detoxification and addiction treatment, has good effects in addiction treatment when used in combination with an addictive substance or an addictive behavior or in the form of a compound preparation prepared with the addictive substance or addictive behavior. It is anticipated that the helpless addiction treatment by lifelong replacement can be solved by course-based cure and the relapse can be prevented, providing a complete and effective drug treatment system for detoxification and addiction treatment.

Imatinib or its derivative imatinib mesylate, also known as Gleevec or ST1571, is a phenylaminopyrimidine derivative, developed by Novartis for the treatment of chronic myeloid leukemia (CIVIL) and approved by the US FDA in 2001. It is known as the first new anti-cancer drug targeting the tumorigenesis mechanism of human beings. Imatinib mainly targets c-Kit, and has a weak effect on the proto-oncogene c-Abl and the platelet-derived growth factor receptor.

In addition, smoking is one of the most serious public health problems in the world today. Tobacco addiction is a chronic and dependent disease. Nicotine, as a main alkaloid component in tobacco, can enter the reward center in the brain and highly bind to the nicotinic acetylcholine receptor, to stimulate the release of dopamine, producing a pleasant feeling. Repeated and persistent smoking behavior leads to nicotine addiction. However, if a smoker quits smoking for a long time, the nicotine in the body is gradually removed, so that the acetylcholine receptor increases, and the transmission of neutral signal is disturbed, causing the feeling of irritation, nausea, headache, and other withdrawal reactions, and thus a strong willing of smoking.

In the process of smoking cessation, the withdrawal symptoms are severe and the relapse rate is extremely high. Once addiction, it is difficult to quit. There are many alternatives to smoking cessation, such as electronic cigarettes, chewable tablets, and nicotine transdermal patches, as well as drugs for smoking cessation such as Bupropion, clonidine, and nortriptyline, they can all lessens the nicotine dependence to a certain extent. However, they have a less sustainable effect and tend to produce side effects. Among them, bupropion and nortriptyline may cause the occurrence of epilepsy and other mental disorders; and the use of clonidine will also have obvious side effects such as sedation and dry mouth. Therefore, there is still a lack of effective drugs for nicotine addiction control and relapse prevention.

The disclosure relates to the development of new therapeutic agents against nicotine addiction. The disclosure adopts a new concept, and finds that imatinib mesylate, a specific and effective drug for smoking cessation and addiction treatment, has good effects in nicotine addiction treatment and can prevent relapse, when used in combination with nicotine or an analogue thereof or in the form of a compound preparation prepared with nicotine or an analogue thereof, providing a complete and effective drug treatment system for smoking cessation treatment.

Moreover, the commonly used analgesics in clinic are mainly opioid analgesics represented by morphine, non-steroidal anti-inflammatory analgesics represented by aspirin and other analgesics. These drugs achieve an analgesic effect by acting on the central or peripheral nervous system, and peripheral tissue inflammation to selectively reduce and eliminate various pains.

The opioid analgesics mainly exert analgesic effect by exciting the opioid receptors. The opioid receptors have a high distribution density in the medial thalamus, spinal glial area, cerebral ventricle and other sites, are associated with the transmission of pain stimuli and the integration and perception of pain signals, and include μ, δ, and κ receptors, all of which are G protein-coupled receptors. The site of analgesic action of the μ receptor is the brain, spinal cord and periphery, the site of analgesic action of the δ receptor is the spinal cord, and the site of analgesic action of the κ receptor is the spinal cord and periphery. Therefore, the opioid analgesics such as morphine exhibit a multi-site analgesic effect, and blocks the peripheral pain transmission and central pain perception, resulting in a potent analgesic effect. Therefore, the opioid analgesics are useful for pains caused by various reasons, but are currently only used in severe cancer pains and short-term applications where other analgesics are ineffective, mainly because consecutively repeated use tends to cause addiction and tolerance, limiting the use of these drugs. Classic opioids suffer from serious addiction and tolerance, are often only used as analgesics for the treatment of severe cancer pains and sharp pains, and are therapeutic agents against severe pain in the three-step treatment of cancers, which have good analgesic effect. New opioids such as oxycodone hydrochloride cause different degrees of addiction when used in moderate and severe cancer pain and chronic non-cancer pain, leading to substance abuse. The severe side effects, addiction, greatly limit their use in clinic.

The non-steroidal anti-inflammatory analgesics represented by aspirin mainly exert an analgesic effect on the periphery, have a weak central analgesic effect, and mainly used for pain caused by tissue damage or inflammation. The non-steroidal anti-inflammatory analgesics relieve pain by inhibiting cyclooxygenase (COX) in peripheral lesions to reduce the synthesis of PGS. They have a good analgesic effect for moderate pains, but a less desirable effect for acute sharp pains, sharp pains caused by severe traumas, smooth muscle colic, and other severe pains.

Other analgesics mainly act on receptors other than opioid receptors, such as NMDA receptor, NK1 receptor, purine and pyrimidine receptors, and cannabinoid receptor, and have a less potent analgesic effect. For example, rotundine has a better effect on chronic persistent dull pain, and the analgesic effect is weaker than pethidine, but stronger than antipyretic antipyretic-analgesic and anti-inflammatory drugs; and lappaconitine can be used as an alternative for mild to moderate pain. However, they cannot meet the current needs in the treatment of severe pain.

Intense pain such as cancer pain, burns, stab wounds, etc., is caused by a variety of factors, and the pain-causing mechanism is complex. The use of exclusively peripheral analgesics cannot prevent the occurrence of severe pains, and central analgesia has to be used. Opioid analgesics such as morphine have powerful analgesic effects and are effective for various pains. They have an irreplaceable position in clinic; however, side effects such as addiction and tolerance greatly limit their use. At present, no desirable drug or compound that can be modified to have a potent analgesic effect and prevent serious side effects such as addiction and tolerance is found, and no desirable method that can eliminate opioid addiction and tolerance and treatment that can reduce or delay the occurrence of drug addiction and tolerance are available in clinic. This makes it difficult to treat severe pains. Moreover, although opioids have a potent analgesic effect, the occurrence of serious side effects such as addiction and tolerance also greatly limit the scope of indications to which opioids with potent analgesic effect are applicable. Therefore, finding a drug or treatment with good analgesic effect and less side effects has become a major problem that needs to be solved urgently in pain treatment and pharmaceutical research in clinic at present.

### SUMMARY

In view of the problems existing in addiction treatment, a first object of the disclosure is to provide new use of imatinib or a derivative thereof in addiction treatment, where imatinib or a derivative thereof is used in combination with an addictive substance or an addictive behavior or prepared with the addictive substance or addictive behavior into a compound preparation to prevent or relieve the mental addiction and withdrawal symptoms after addiction and cessation, and prevent relapse.

In view of the problems existing in the treatment of addiction to nicotine or an analogue thereof in addictive substances, a second object of the disclosure is to provide new use and a new preparation and dosage form of imatinib or a derivative thereof in the treatment of addiction to nicotine or an analogue thereof, where imatinib or a derivative thereof is used in combination with the addictive substance nicotine or prepared with the addictive substance nicotine into a compound preparation to prevent or relieve the mental addiction and withdrawal symptoms after addiction and cessation, and prevent relapse.

In view of the side effects addiction and tolerance existing in analgesia with analgesic drugs, a third object of the disclosure is to provide new use of imatinib or a derivative thereof in analgesic drugs, where imatinib or a derivative thereof is used in combination with an analgesic drug at a ratio of 2:1 or prepared with the analgesic drug at a ratio of 2:1 into a compound preparation to prevent or relieve the addiction or tolerance to the analgesic drug without affecting the analgesic effect of the drug, and to widen the scope of indications to which the analgesic drug is applicable.

To achieve the first object of the disclosure, the following studies are carried out, including

treatment or prevention of addiction, mechanism, dose-effect relationship, and control of relapse after withdrawal, with imatinib or a derivative thereof administered in combination with or in the form of a compound preparation with an addictive substance or an addictive behavior.

To demonstrate that imatinib or a derivative thereof, when administered in combination with or in the form of a compound preparation with an addictive substance (morphine, cocaine, and alcohol), can prevent or treat addiction formed when the addictive substance is administered alone or during an addictive behavior and have a dose-dependent effect (where in the combined administration, imatinib or a derivative thereof, and the addictive substance are administered through different routes at an interval of 30 min; and in the administration in the form of a compound preparation, imatinib or a derivative thereof is mixed with the addictive substance and then administered), the following experiments are carried out in the disclosure.

(1) 60 min after acute morphine administration, the changes of c-kit activity in the mesolimbic dopamine system, including VTA, nucleus accumbens, amygdala, hippocampus, and prefrontal cortex are observed, the changes in phosphorylation level of c-kit are observed by immunohistochemistry combined with western-blot, and the distribution of activated cells is observed by immunofluorescence co-labeling to determine the new molecular mechanism of morphine addiction and the mechanism of action of imatinib mesylate in controlling addiction. Further, a drug-induced CPP model is used to observe the prevention for development of drug-included CPP, the dose-effect and the mechanism of action of imatinib mesylate. 1, 5, 10, 20, and 30 mg/kg of imatinib mesylate are intraperitoneally given to experimental rats; after 30 min, 5 mg/kg, and 10 mg/kg of morphine (i.e., combined administration) are subcutaneously injected. Alternatively, 1, 5, 10, 20, and 30 mg/kg of imatinib mesylate are respectively prepared with 5 mg/kg, and 10 mg/kg of morphine into a mixed injection and then administered. The classic conditioned place preference of rats for evaluating addiction is used to determine the influence of imatinib mesylate on the development of morphine-induced conditioned place preference in rats.

(2) Acute administration and a drug-induced CPP model are used to observe the prevention for development of cocaine-induced CPP, the dose-effect and the mechanism of action of imatinib mesylate.

(3) Acute administration and a drug-induced CPP model are used to observe the prevention for development of ethanol-induced CPP, the dose ratio and the mechanism of action of imatinib mesylate. The dose of ethanol is 0.5, and 0.75 g/kg; and the dose of imatinib mesylate is 1, 5, 10, 15, 20, and 30 mg/kg.

(4) A drug-induced CPP model is used to observe the dose-effect of imatinib mesylate of 1, 5, 10, 20, and 30 mg/kg on the drug-seeking behavior after morphine addiction in rats after induction by unconditioned stimulus. After the development of morphine-induced CPP in rats, the rats are exposed to a CPP chamber, and then intraperitoneally administered with 1, 5, 10, 20, and 30 mg/kg of imatinib mesylate; and the CPP score is tested on the first day after drug intervention to observe the influence on the drug-seeking behavior, and the CPP score is further tested on the 7th day after drug intervention.

(5) A drug-induced CPP model is used to observe the dose-effect of imatinib mesylate of 1, 5, 10, 20, and 30 mg/kg on the drug-seeking behavior after cocaine addiction in rats after induction by unconditioned stimulus.

(6) A drug-induced CPP model is used to observe the dose-effect of imatinib mesylate of 1, 5, 10, 15, 20, and 30 mg/kg on the drug-seeking behavior after ethanol addiction in rats after induction by unconditioned stimulus.

(7) A drug-induced CPP model is used to observe the effect of 30 mg/kg imatinib mesylate administered in combination with environmental clue or with unconditioned stimulus (different doses), or directly on the drug-seeking behavior and relapse after morphine addiction in rats. After the development of morphine-induced CPP in rats, the rats are induced with environmental clue or unconditioned stimulus, or no induction is performed, and then imatinib mesylate is given. The induction with environmental clue is to place the addicted rats in a drug-paired side of a CPP chamber, to induce the addiction memory, and then imatinib mesylate is given. The induction by unconditioned stimulus is to given morphine (3, 4 mg/kg) that is, morphine and imatinib mesylate are used in combination, and a group with drug combination and a group with compound preparation are included. No induction means that the rats are not transferred to a CPP chamber, and not given morphine, but directly treated with imatinib mesylate. The CPP score is tested on the 1st day after the drug intervention to observe the effect on the drug-seeking behavior, and the CPP score is further tested on the 7th day after drug intervention. On the 9th day after the intervention, all rats are challenged with a low dose (3 mg/kg) of morphine and the effect on relapse is observed.

(8) A drug-induced CPP model is used to observe the effect of 30 mg/kg imatinib mesylate administered in combination with environmental clue or substance clue (various doses of cocaine), or directly on the drug-seeking behavior and relapse after cocaine addiction in rats.

(9) A drug-induced CPP model is used to observe the effect of imatinib mesylate of 15 and 30 mg/kg administered in combination with environmental clue or substance clue (various doses of ethanol), or directly on the drug-seeking behavior and relapse after ethanol addiction in rats.

(10) A drug-induced CPP model is used to observe the effect of different doses of imatinib mesylate on withdrawal symptoms upon combined administration of imatinib mesylate and morphine or administration of a compound preparation of imatinib mesylate and morphine, after the development of CPP.

(11) A drug-induced CPP model is used to observe the effect of different doses of imatinib mesylate on withdrawal symptoms upon combined administration of imatinib mesylate and cocaine or administration of a compound preparation of imatinib mesylate and cocaine, after cocaine-induced CPP is developed.

(12) A drug-induced CPP model is used to observe the effect of different doses of imatinib mesylate on withdrawal symptoms upon combined administration of imatinib mesylate and ethanol or administration of a compound preparation of imatinib mesylate and ethanol, after the development of CPP.

(13) A sensitization model is used to explore the effect of different doses of imatinib mesylate on the development and expression of morphine sensitization in rats.

(14) A sensitization model is used to explore the effect of different doses of imatinib mesylate on the development and expression of cocaine sensitization in rats.

(15) A sensitization model is used to explore the effect of different doses of imatinib mesylate on the development and expression of ethanol sensitization in rats.

(16) A drug-induced CPP model is used to explore the dose-effect of imatinib mesylate on the development of conditioned place preference in rats on high-sugar and high-fat food.

(17) A drug-induced CPP model is used to explore the dose-effect of imatinib mesylate on the reconsolidation and relapse after the development of conditioned place preference in rats on high-sugar and high-fat food.

(18) A rat gambling task is used to explore the dose-effect of imatinib mesylate on the gambling behavior.

After acute morphine or cocaine administration, the results of immunohistochemistry, western-blot and immunofluorescence co-labeling show that the c-kit receptor, and then downstream ERK, AKT, and PKMzeta signaling molecules in neurons in nucleus accumbens instead of in other brain regions are specifically activated; imatinib mesylate inhibits the drug-seeking behavior by blocking the c-kit receptor and its signaling pathway, thereby preventing morphine addiction and controlling relapse. Also, imatinib or a derivative thereof administered in combination with or in the form of a compound preparation with morphine at a certain dose ratio can prevent and treat morphine addiction and relapse after withdrawal.

Imatinib mesylate has different blocking effects on the development of morphine- and cocaine-induced CPP, and memory reconsolidation after development. Conditioned place preference and sensitization are developed in rats not receiving imatinib mesylate; and after imatinib mesylate is administered, the development of conditioned place preference and sensitization can be prevented when the dose of imatinib mesylate is in the range of (10-30 mg/kg) and at a ratio to the dose of morphine or cocaine upon addiction of 2:1 or greater, regardless of administration in combination or in the form of a compound preparation. After conditioned place preference and sensitization are developed, the direct administration of imatinib mesylate of 20, and 30 mg/kg can block the drug-seeking behavior of rats after addiction, but the rats are still primed, that is, the relapse cannot be controlled. When induced by environmental clue, imatinib mesylate of 20 and 30 mg/kg can block the drug-seeking behavior of rats after addiction, and only 30 mg/kg can prevent the relapse. When induced by unconditioned stimulus, the relapse can be effectively controlled, and the drug-seeking behavior can be inhibited and prevented from being primed only when the effective ratio of dose of morphine and cocaine to that used for training CPP is less than 1:3. When the dosage of morphine or cocaine is 3 mg/kg, 20 and 30 mg/kg of imatinib mesylate can prevent the drug-seeking behavior and relapse in rats after addiction. When the dosage of morphine or cocaine is 4 mg/kg, 20 and 30 mg/kg of imatinib mesylate can only inhibit the drug-seeking behavior, and cannot prevent relapse.

After acute ethanol administration, the results of immunohistochemistry, western-blot and immunofluorescence co-labeling show that the c-kit receptor in neurons in nucleus accumbens instead of in other brain regions is specifically activated, and imatinib mesylate (10, 15, 20, and 30 mg/kg) have different inhibition on the development of ethanol-induced conditioned place preference and memory reconsolidation after development. Conditioned place preference and sensitization are developed in rats not receiving imatinib mesylate; and after imatinib mesylate is administered, the development of conditioned place preference can be prevented when the dose ratio of imatinib mesylate to ethanol is greater than or equal to 1:50, regardless of administration in combination or in the form of a compound preparation. After conditioned place preference is developed, the direct administration of imatinib mesylate can block the drug-seeking behavior of rats after addiction when the dose ratio of imatinib mesylate to ethanol is 1:50 or greater; but the rats are still primed, that is, the relapse cannot be controlled. When induced by environmental clue, imatinib mesylate at a dose ratio to ethanol of 1:50 or greater can block the drug-seeking behavior of rats after addiction, and only a dose of 30 mg/kg can prevent the relapse. When induced by unconditioned stimulus, drug-seeking behavior and relapse in rats after addiction can be controlled when the dose of ethanol is at a ratio to the dose for training CPP of 1:3 or less, and the dose of imatinib mesylate is at a ratio to the dose of ethanol for training CPP of 1:50 or greater. When the dose of ethanol as unconditioned stimulus is 0.5 g/kg, 20 and 30 mg/kg of imatinib mesylate can only inhibit the drug-seeking behavior, and cannot prevent relapse. Therefore, imatinib mesylate inhibits the drug-seeking behavior by blocking the c-kit receptors, to achieve the effect of preventing alcohol addiction and controlling relapse. Similarly, imatinib or a derivative thereof administered in combination with or in the form of a compound preparation with ethanol at a certain dose ratio can prevent and treat ethanol addiction and relapse after withdrawal.

For an addictive behavior, the effect of imatinib mesylate was similar to that for morphine, indicating that the c-kit receptor is universal as a target for addiction treatment with a drug.

In clinical use, the equivalent dose conversion between human beings and animals based on body surface area can refer to “Experimental Methodology of Pharmacology” edited by Professor Xu Shuyun.

For example: the dose for rats is X mg/kg, and the clinical dose for adults is:

Clinical dose for adults=X mg/kg×0.2 kg/0.018=11.1X mg/day. Therefore, the doses (1, 5, 10, 20, and 30 mg/kg) of imatinib mesylate for rats can be converted into the clinical doses for adults, that is, 11, 55, 110, 220, and 330 mg/day.

According to the issued data of clinical trial of the marketed drug Gleevec imatinib (Gleevec®), 400 mg/day/70 kg has few side effects during clinical use. Therefore, 100-400 mg/day/70 kg is used as a clinical dose of imatinib mesylate for use with an addictive substance at a certain ratio. This dose is in a safe range of clinical dose at present (FIG. 22).

Based on the above studies, the disclosure provides the following technical solutions:

1. A pharmaceutical preparation in which imatinib mesylate and a derivative thereof are administered in combination with or in the form a compound preparation with an addictive substance at a certain ratio, for the prevention and treatment of various addictions, and the control of relapse.

2. Use of imatinib mesylate and a derivative thereof in combination with an addictive behavior, where (1) use of imatinib mesylate and a derivative thereof compounded with or in combination with an addictive food can prevent and treat addiction, and control relapse; and (2) after a gambling behavior is induced by environmental clue, the administration of imatinib mesylate and a derivative thereof can improve the gambling behavior.

3. The addictive substance refers to (1) narcotic drugs, including opioids, cocaine, and cannabis, where opioids include opium derived from natural sources, the active ingredient morphine extracted therefrom heroin products obtained by processing the active ingredient, and synthetic products with similar effects of opioids; (2) psychotropic drugs, including sedative hypnotics, anxiolytic barbiturates, central stimulant amphetamine, and hallucinogen lysergide; and (3) addictive substances such as ethanol, tobacco and volatile organic solvents; and where when they are used as an unconditioned stimulus, the dose is at a ratio to the dose for training CPP of 1:3 or less.

4. The addictive behavior refers to all addictive behaviors such as (1) additive food, including high-fat food, sweets, chocolate and other delicious foods; and (2) gambling addiction and Internet addiction.

5. The dose of imatinib or a derivative thereof is 100-400 mg/day.

6. Imatinib mesylate used for the treatment and prevention of relapse of substance addiction needs to be used in combination or in the form of a compound preparation with the addictive substance.

7. Imatinib mesylate used for the treatment and prevention of relapse of behavior addiction needs to be used in combination or in the form of a compound preparation with exposure to an addictive behavior-related clue.

8. The ratio means that imatinib mesylate is effective in the prevention and treatment of addiction and the control of relapse only when imatinib mesylate is at a certain ratio to the dose of a substance for developing addiction, where imatinib mesylate is effective only when the dose ratio of imatinib mesylate to morphine or cocaine is 2:1 or greater; and imatinib mesylate is effective only when the dose ratio of imatinib mesylate to ethanol is 1:50 or greater.

9. Imatinib or a derivative thereof can be used in combination with the addictive substance or addictive behavior within the dose range or in the form of a compound single preparation prepared with the addictive substance or addictive behavior within the dose range.

10. The administration in combination or in the form of a compound preparation means that imatinib or a derivative thereof is used in combination with opioids at a certain ratio, or used in the form of an injection, an infusion, pills, an subcutaneous implant, a tablet, a powder, granules, a capsule, a powder, an oral liquid, a sustained-release preparation, a tincture, a suppository, a patch and other dosage forms prepared with opioids at a certain ratio; and the administration in combination or in the form of a compound preparation at a certain ratio also includes all pharmaceutical preparations or combinations at such a ratio useful for addiction treatment.

To achieve the second object of the disclosure, the following studies are carried out, including

(1) Dose ratio-effect and mechanism of imatinib or a derivative thereof administered in combination with or in the form of a compound preparation with the addictive substance nicotine in the treatment or prevention of addiction

To confirmed that imatinib or a derivative thereof administered in combination with or in the form of a compound preparation with nicotine can prevent or treat addiction developed when nicotine is administered alone and have a dose ratio-dependent effect, the following experiments are carried out in the disclosure. The rats are intraperitoneally administered with 1, 5, 10, 20, or 30 mg/kg imatinib mesylate, and then subcutaneously injected with 0.25, or 5 mg/kg nicotine. Alternatively, 1, 5, 10, 20, or 30 mg/kg imatinib mesylate is prepared into a mixed injection with 0.25, or 5 mg/kg nicotine, and then administered. The classic conditioned place preference (CPP) model of rats for evaluating addiction is used to determine the dose ratio-effect of imatinib mesylate in the prevention of development of nicotine-induced CPP in rats. Further, 60 min after acute nicotine administration, the changes of c-kit activity in the mesolimbic dopamine system including VTA, nucleus accumbens, amygdala, hippocampus, and prefrontal cortex and the changes in c-kit phosphorylation level are observed, and the downstream activated target molecules are determined by multicolor immunofluorescence co-labeling, to determine the new molecular mechanism of nicotine addiction and the mechanism of action of imatinib mesylate in controlling addiction.

(2) A drug-induced CPP model is used to observe the dose-effect of imatinib mesylate of 1, 5, 10, 20, and 30 mg/kg on the drug-seeking behavior after nicotine addiction in rats after induction by unconditioned stimulus. After the development of nicotine-induced CPP in rats, the rats are changed with a small dose of nicotine, and then intraperitoneally administered with 1, 5, 10, 20, or 30 mg/kg imatinib mesylate (combined administration); or a mixture of imatinib mesylate and nicotine (compound preparation) is administered, the CPP score is tested on the first day after drug intervention to observe the effect on the drug-seeking behavior, and the CPP score is further tested on the 7th day after drug intervention.

(3) A drug-induced CPP model is used to observe the effect of 30 mg/kg imatinib mesylate administered in combination with environmental clue or unconditioned stimulus (different doses), or directly on the drug-seeking behavior and relapse after nicotine addiction in rats. After the development of nicotine-induced CPP in rats, induction with environmental clue or with unconditioned stimulus, or no induction is performed, and imatinib mesylate is given. The induction with environmental clue is to place the addicted rats in a drug-paired side of a CPP chamber, to induce the addiction memory, and then imatinib mesylate is given. The induction by unconditioned stimulus is to given nicotine (0.1, and 0.15 mg/kg), that is, nicotine and imatinib mesylate are used in combination, and a group with drug combination and a group with compound preparation are included. No induction means that the rats are not transferred to a CPP chamber, and not given with nicotine, but directly treated with imatinib mesylate. The CPP score is tested on the 1st day after the drug intervention to observe the effect on the drug-seeking behavior, and the CPP score is further tested on the 7th day after drug intervention. On the 9th day after the intervention, all rats are challenged with a low dose (0.1 mg/kg) of nicotine and the effect on relapse is observed.

(4) A drug-induced CPP model is used to observe the effect of different doses of imatinib mesylate on withdrawal symptoms upon combined administration of imatinib mesylate with nicotine or administration of a compound preparation of imatinib mesylate with nicotine, after nicotine-induced CPP is developed.

To confirm that the administration of imatinib or a derivative thereof in combination with or in the form of a compound preparation with the addictive substance nicotine can treat nicotine withdrawal symptoms and have a dose ratio-dependent effect, the following experiments are carried out in the disclosure. A CPP model is used. After the rats become addicted to nicotine at a dose of 0.25 or 0.5 mg/kg, 1, 5, 10, 20, or 30 mg/kg of imatinib mesylate is intraperitoneally injected on the following day, and 0.1 mg/kg of nicotine is subcutaneously injected 30 min later; or 1, 5, 10, 20, or 30 mg/kg of imatinib mesylate is prepared into a mixed injection with 0.15 mg/kg of nicotine and then administered. After 60 min, a spontaneous activity detecting apparatus is used and the dose-effect of imatinib mesylate in the treatment of nicotine withdrawal symptoms according to the changes of locomotor activity in rats after nicotine withdrawal.

(5) In clinical use, the dose of imatinib or a derivative thereof used with nicotine is 100-400 mg/day.

A CPP model is used, and imatinib mesylate at a dose ratio to nicotine of 40:1 or greater can control nicotine addiction and relapse and alleviate the withdrawal symptoms after nicotine addiction, regardless of administration in combination or in the form of a compound preparation, where the minimum dose of imatinib mesylate is 10 mg/kg. In clinical use, the equivalent dose conversion between human beings and animals based on body surface area can refer to “Experimental Methodology of Pharmacology” edited by Professor Xu Shuyun.

For example: the dose for rats is X mg/kg, and the clinical dose for adults is:

Clinical dose for adults=X mg/kg×0.2 kg/0.018=11.1X mg/day. Therefore, the doses (1, 5, 10, 20, and 30 mg/kg) of imatinib mesylate for rats can be converted into the clinical doses for adults, that is, 11, 55, 110, 220, and 330 mg/day.

According to the issued data of clinical trial of the marketed drug Gleevec imatinib (Gleevec®), 400 mg/day/70 kg has few side effects during clinical use. Therefore, 100-400 mg/day/70 kg is used as a clinical dose of imatinib mesylate.

Based on the above studies, the disclosure provides the following technical solutions:

use of imatinib or a derivative thereof with nicotine or an analogue thereof in the preparation of drugs including drugs for the prevention and treatment of nicotine addiction, and for the control of relapse. The drug is used in the form of a combination or a compound preparation of imatinib or a derivative thereof with nicotine or an analogue thereof. The ratio of imatinib or a derivative thereof to nicotine or an analogue thereof is 40:1 or greater based on the active ingredients. Clinically, the dose of imatinib or a derivative thereof is 100-400 mg/day. When the nicotine addiction memory is aroused, the dose used needs to be at a ratio to the dose for developing addiction of 3:10 or less based on the active ingredient, then imatinib or a derivative thereof can effectively prevent and treat nicotine addiction and control relapse. The drug is suitably prepared into the following dosage forms, including an injection, an infusion, a subcutaneous implant, pills, a tablet, a powder, granules, a capsule, a powder, an oral liquid, a sustained-release preparation, a tincture, a suppository, and a patch.

To achieve the third object of the disclosure, the following studies are carried out, including

(1) Dose ratio-effect and mechanism of imatinib or a derivative thereof administered in combination with or in the form of a compound preparation with morphine in the treatment or prevention of opioid addiction

To confirm that imatinib or a derivative thereof administered in combination with or in the form of a compound preparation with morphine can prevent or treat addiction developed when morphine is administered alone and have a dose ratio-dependent effect, the following experiments are carried out in the disclosure. A pain model is used. After morphine analgesia, the mice are immediately transferred to a conditioned place preference (CPP) training device. Whether CPP is formed after morphine analgesia is observed to confirmed that morphine analgesia can lead to addiction, and the dose ratio-effect of imatinib mesylate on the prevention of CPP development after morphine analgesia is observed. In the dose ratio-effect experiment of imatinib mesylate with morphine, the rats are intraperitoneally administered with 1, 5, 10, 20, or 30 mg/kg of imatinib mesylate, and then subcutaneously injected with 5, or 10 mg/kg of morphine; or 1, 5, 10, 20, or 30 mg/kg of imatinib mesylate is prepared into a mixed injection with 5, or 10 mg/kg of morphine into a mixed injection and then administered. The classic conditioned place preference (CPP) model of rats for evaluating addiction is used to determine the dose ratio-effect of imatinib mesylate in the prevention of development of morphine-induced CPP in rats. Further, 60 min after acute morphine administration, the changes of c-kit activity in the mesolimbic dopamine system including VTA, nucleus accumbens, amygdala, hippocampus, and prefrontal cortex are observed, the changes in phosphorylation level of c-kit are observed by immunohistochemistry combined with western-blot, the distribution of activated cells is observed by immunofluorescence co-labeling and the downstream activated target molecules are determined by multicolor immunofluorescence co-labeling, to determine the new molecular mechanism of morphine addiction and the mechanism of action of imatinib mesylate in controlling addiction.

The results show that in the pain model, CPP is developed in mice after morphine analgesia, and imatinib mesylate at a different dose ratio to morphine (2:1 or greater) has different inhibitions on the development of morphine-induced CPP. In mice not receiving imatinib mesylate, conditioned place preference is developed. Regardless of administration in combination or in the form of a compound preparation, after imatinib mesylate is administered, 10, 20, and 30 mg/kg, but not 1 and 5 mg/kg, of imatinib mesylate can prevent the development of CPP trained by 5 mg/kg of morphine; and 20 and 30 mg/kg, but not 1, 5, and 10 mg/kg, of imatinib mesylate can prevent the development of CPP trained by 10 mg/kg of morphine, indicating that imatinib mesylate at a dose ratio to morphine of 2:1 or greater can prevent morphine addiction. After acute morphine administration, the results of immunohistochemistry, western-blot and multi-color immunofluorescence co-labeling show that the c-kit receptor, and then multiple downstream signaling molecules such as PKC, PI3K-AKT, ERK, and other regulatory kinases, protein expressions, gene expressions, and regulation and initiation of the process of morphine reward, memory and neuroplasticity in neurons in nucleus accumbens instead of in other brain regions are specifically activated. Imatinib mesylate inhibits multiple signaling molecules such as PKC, PI3K-AKT, ERK, and other regulatory kinases, protein expressions, gene expressions, and regulation and initiation of the process of morphine reward, memory and neuroplasticity by blocking the c-kit receptor, to achieve the effect of preventing morphine addiction.

(2) Dose ratio-effect and mechanism of imatinib or a derivative thereof administered in combination with or in the form of a compound preparation with morphine in the treatment or prevention of opioid addiction

To confirm that imatinib or a derivative thereof administered in combination with or in the form of a compound preparation with morphine can prevent or treat tolerance developed when morphine is administered alone and have a dose ratio-dependent effect, the following experiments are carried out in the disclosure. A hot-plate pain model is used. The rats are intraperitoneally administered with 1, 5, 10, 20, or 30 mg/kg of imatinib mesylate, and then subcutaneously injected with 5, or 10 mg/kg of morphine; or 1, 5, 10, 20, or 30 mg/kg of imatinib mesylate is prepared into a mixed injection with 5, or 10 mg/kg of morphine into a mixed injection and then administered. The dose ratio-effect of imatinib mesylate on the prevention and treatment of development of morphine tolerance in rats is determined according to a heat pain threshold.

The results show that in the hot-plate pain model, imatinib mesylate at a different dose ratio to morphine (2:1 or greater) has different inhibition on the development of morphine tolerance. In mice not receiving imatinib mesylate, morphine tolerance is developed. Regardless of administration in combination or in a compound preparation, after imatinib mesylate is administered, 10, 20, and 30 mg/kg, but not 1, and 5 mg/kg, of imatinib mesylate can prevent the development of morphine tolerance induced by 5 mg/kg morphine; and 20 and 30 mg/kg, but not 1, 5, and 10 mg/kg, of imatinib mesylate can prevent the development of CPP trained by 10 mg/kg of morphine, indicating that imatinib mesylate at a dose ratio to morphine of 2:1 or greater can prevent morphine addiction.

(3) Imatinib or a derivative thereof administered in combination with or in the form of a compound preparation with morphine at a certain dose ratio can prevent the side effects associated with central analgesia of morphine without affecting the analgesic effect.

To confirm that imatinib or a derivative thereof administered in combination with or in the form of a compound preparation with morphine at a certain dose ratio have no impact on the analgesic effect, the following experiments are carried out in the disclosure. In an experiment with combined administration, 1, 5, 10, 20, or 30 mg/kg of imatinib mesylate is intraperitoneally administered, and then 10 mg/kg of morphine is subcutaneously injected 30 min later. In an experiment with administration in the form of a compound preparation, 1, 5, 10, 20, or 30 mg/kg of imatinib mesylate is respectively mixed with 10 mg/kg of morphine, and then subcutaneously injected immediately. The classical pain models (hot-plate pain model, acetic acid induced writhing model, formalin induced pain model, and CFA-induced chronic plantar pain model) are used to observe effects of imatinib mesylate administered in combination with or in the form of a compound preparation with morphine at a certain dose ratio on various test indices.

The results show that regardless of the dose ratio, imatinib mesylate administered in combination with or in the form of a compound preparation with morphine has no obvious impact on various test indices.

(4) In clinical use, the dose of imatinib or a derivative thereof used with morphine is 100-400 mg/day.

A morphine tolerance model is used; and regardless of administration in combination or in the form of a compound preparation, imatinib mesylate at a dose ratio to morphine of 2:1 or greater can prevent the development of morphine tolerance, and increase the heat pain threshold in rats; however, a dose ratio lower than 2:1 has no such an effect, where the minimum dose of imatinib mesylate is 10 mg/kg. A pain model is used, CPP is development after morphine analgesia, and imatinib mesylate at a different dose ratio to morphine (2:1 or greater) has different inhibition on the development of morphine tolerance; however, a dose ratio lower than 2:1 has no such an effect, where the minimum dose of imatinib mesylate is 10 mg/kg. These suggest that a composition of imatinib mesylate with morphine at a dose ratio of 2:1 or greater can prevent the occurrence of morphine addiction, and also the occurrence of the side effect tolerance, where the minimum dose of imatinib mesylate is 10 mg/kg. In clinical use, the equivalent dose conversion between human beings and animals based on body surface area can refer to “Experimental Methodology of Pharmacology” edited by Professor Xu Shuyun.

For example: the dose for rats is X mg/kg, and the clinical dose for adults is:

Clinical dose for adults=X mg/kg×0.2 kg/0.018=11.1X mg/day. Therefore, the doses (1, 5, 10, 20, and 30 mg/kg) of imatinib mesylate for rats can be converted into the clinical doses for adults, that is, 11, 55, 110, 220, and 330 mg/day.

According to the issued data of clinical trial of the marketed drug Gleevec imatinib (Gleevec®), 400 mg/day/70 kg has few side effects during clinical use. Therefore, 100-400 mg/day/70 kg is used as a clinical dose of imatinib mesylate for use with opioid analgesics at a certain ratio.

Based on the above studies, the disclosure provides the following technical solutions:

use of imatinib or a derivative thereof and an analgesic agent in the preparation of drugs. The use includes: (1) use of a composition comprising imatinib or a derivative thereof and an analgesic agent in the preparation of drugs for the treatment of pain; and (2) use of a composition comprising imatinib or a derivative thereof and an analgesic agent in the preparation of drugs for preventing tolerance to and the side effect addiction to the analgesic drug.

In the use, the ratio of imatinib or a derivative thereof to the analgesic agent in the composition is 2:1 or greater based on the active ingredients. Clinically, the dose of imatinib or a derivative thereof is 100-400 mg/day.

The analgesic agent includes addictive opioids that act on the central analgesia system to produce analgesic effects, such as morphine, codeine, pethidine, fentanyl, methadone, oxycodone, hydromorphone, nalbuphine, and marijuana, and various non-opioid addictive compounds or their salts.

The pain refers to the indications and other various types of acute and chronic pain that can be suitably treated by the administration of the analgesic drug alone.

According to the composition comprising imatinib or a derivative thereof and an analgesic agent, imatinib or a derivative thereof and the analgesic agent can be administered simultaneously, separately or sequentially.

The drug is suitably prepared into the following dosage forms, including an injection, an infusion, a subcutaneous implant, pills, a tablet, a powder, granules, a capsule, a powder, an oral liquid, a sustained-release preparation, a tincture, a suppository, and a patch.

### Beneficial Effects of the Disclosure

Firstly, for the composition of imatinib or a derivative thereof and an addictive substance according to the disclosure, imatinib or a derivative thereof and the addictive substance are prepared into various compound preparations or combined preparations and used for addiction treatment for the first time from the perspective of clinically safe dose, dose ratio, new intervention method and new mechanism of action, thus well meeting the clinical needs, and achieving a great advancement in the addiction prevention and control on this basis. Imatinib and the addictive substance have no interactive groups in their molecular structures, so the administration of imatinib or a derivative thereof in combination with or in the form of a compound preparation with the addictive substance is feasible, effective, highly safe, and highly clinically controllable in addiction treatment, making a substantial progress in addiction treatment. The dose of imatinib mesylate (100-400 mg/day/70 kg) used in the disclosure has few side effects during clinical use, is within a safe dose range for clinical use, has good clinical effectiveness and safety, and can be widely used in the clinical treatment of patients with various acute and chronic addiction, to solve the bottleneck problem currently existing in clinical addiction treatment. Compared with the existing research, the disclosure possesses the following substantial progress and differences:

New dose: With respect to the dose-effect, it is found in the disclosure that a low dose of 5 mg/kg can be used in the treatment and prevention of morphine tolerance in rats, but not in the prevention and treatment of addiction and relapse after withdrawal; and a dose of 10 mg/kg or higher can prevent and treat opioid tolerance and addiction, and can also successfully prevent addiction to opioid and other substances and relapse after withdrawal. This typical dose-effect is similarly exemplified in the clinical use of the analgesic-antipyretic drug aspirin. For example, a low dose of aspirin is used for antithrombus, a medium dose provides an antipyretic and analgesic effect, and a high dose provides an anti-inflammatory and anti-rheumatic effect. The antithrombotic, antipyretic and analgesic, and anti-inflammatory and anti-rheumatic effects of the drug are completely different and have substantial differences. Therefore, the dose-effect of the disclosure (10 mg/kg or higher for rats, equivalent dose for other species, and 100 mg/day or higher for clinical use) is a substantial new discovery of imatinib mesylate in the addiction treatment. Administration of imatinib mesylate in this dose range in combination with or in the form of various compound preparations with an addictive drug to prevent and treat addiction and relapse after withdrawal indicates usefulness of the drug in different dose ranges to different indications, making the disclosure have substantially different applications and advancement over earlier inventions.

New ratio: The disclosure finds that imatinib mesylate is effective in the prevention and treatment of addiction and drug-induced CPP only when there is a certain dose ratio. Imatinib mesylate is effective only when the dose ratio to morphine or cocaine is 2:1 or greater; and imatinib mesylate is effective only when the dose ratio to ethanol is 1:50 greater. In addition, a certain dose ratio is also needed for unconditioned induction. For example, the effective ratio of the dose of morphine, cocaine, and ethanol to the dose for training CPP is 1:3 or less.

New treatment strategy: In the disclosure, a new intervention method, that is, drug treatment and intervention after an unconditioned cue-induced reconsolidation process, is used. This intervention method is easier to operate in clinical practice and has more effective therapeutic effect.

New mechanism: The disclosure has significantly different mechanism of action.

Imatinib mesylate inhibits multiple signaling molecules such as PKC, PI3K-AKT, ERK, and other regulatory kinases, protein expressions, gene expressions, and regulation and initiation of the process of morphine reward, memory and neuroplasticity by blocking the c-kit receptor, thereby blocking the various effects of morphine and achieving the effect of preventing and treating morphine addiction.

Secondly, for the composition of imatinib or a derivative thereof and nicotine or an analogue thereof, a combination or a compound preparation of imatinib or a derivative thereof with nicotine at a dose ratio of 40:1 or greater can effectively control nicotine addiction and relapse and alleviate the withdrawal symptoms after addiction compared with the traditional single drug, thus effectively solving the problem of nicotine addiction treatment in clinical patients. It is also found that when the dose ratio of imatinib mesylate to nicotine is less than 40:1, nicotine addiction cannot be controlled; and when the dose ratio of imatinib mesylate to nicotine is 40:1 or greater, nicotine addiction can be successfully prevented, and the nicotine withdrawal symptoms can be alleviated. This typical dose-effect is similarly exemplified in the clinical use of the antipyretic drug aspirin. For example, a low dose of aspirin is used for antithrombus, a medium dose provides an antipyretic and analgesic effect, and a high dose provides an anti-inflammatory and anti-rheumatic effect. The antithrombotic, antipyretic and analgesic, and anti-inflammatory and anti-rheumatic effects of the drug against clinical indications are completely different and have substantial differences. Moreover, the disclosure finds that in the treatment of relapse of nicotine addiction with imatinib mesylate, if the ratio of the nicotine addiction memory arousing dose to the dose for training nicotine addiction is 3:10 or greater, the dose of imatinib mesylate for preventing nicotine addiction cannot prevent and treat relapse of nicotine addiction; and if the ratio is less than 3:10, the dose of imatinib mesylate for preventing nicotine addiction can prevent and treat relapse of nicotine addiction. Therefore, the dose ratio-effect of imatinib mesylate in treatment (where dose ratio of imatinib mesylate to nicotine in rats is 40:1 or higher, equivalent dose is used for other species, and the clinical dose is 100 mg/day or higher) and the dose ratio-effect in arousing nicotine addiction memory (the ratio of the dose of arousing nicotine addiction memory in rats to the dose for training nicotine addiction is 3:10 or less, and equivalent dose is used for other species) are substantially new discoveries of imatinib mesylate in the treatment of nicotine addiction. Administration of imatinib mesylate in combination with or in the form of various compound preparations with nicotine in this range of dose ratio to prevent and treat nicotine addiction and relapse after withdrawal indicates usefulness of the drug in different dose ranges to different indications.

1) Substantial progress in dose: The disclosure finds that the effective dose ratio of imatinib mesylate for preventing and treating nicotine addiction and relapse needs to be greater than or equal to 40:1, and the ratio of the nicotine addiction memory arousing dose to the dose for developing nicotine addiction memory needs to be less than 3:10.

2) As for the treatment strategy, after imatinib or a derivative thereof and nicotine or an analogue thereof are combined or prepared into a compound preparation at a certain ratio, the treatment mode and mechanism are completely different, leading to a more effective therapeutic effect, easy operation and convenient administration, avoiding the inconvenience in administration of the drug to patients. Thus, the applicability is much better.

3) Compared with the existing drugs for the treatment of nicotine addiction, the disclosure has significantly different mechanism of action. Imatinib mesylate inhibits multiple signaling molecules such as PKC, PI3K-AKT, ERK, and other regulatory kinases, protein expressions, gene expressions, and regulation and initiation of the process of nicotine reward, memory and neuroplasticity by blocking the c-kit receptor, thereby blocking the various effects of nicotine and achieving the effect of preventing and treating addiction and relapse.

In the disclosure, imatinib or a derivative thereof and the addictive substance nicotine or an analogue thereof are proposed to be prepared into various compound preparations or combined preparations at a certain ratio and used for addiction and relapse treatment for the first time from the perspective of dose ratio, new intervention method and new mechanism of action, thus well meeting the clinical needs, and achieving a great advancement in the addiction prevention and control on this basis. Imatinib and nicotine have no interactive groups in their molecular structures, so the administration of imatinib or a derivative thereof in combination with or in the form of a compound preparation with nicotine proposed in the disclosure is feasible, effective, highly safe, and highly clinically controllable in nicotine treatment, making a substantial progress in nicotine addiction treatment.

Thirdly, for the composition of imatinib or a derivative thereof and an analgesic agent according to the disclosure, compared with the traditional single agent, a combination or a compound preparation of imatinib or a derivative thereof with the analgesic agent at a dose ratio of 2:1 or greater can effectively exert an analgesic effect of the analgesic agent and also effectively prevent the serious side effects of opioids, without affecting the analgesic effect of opioids. This effectively solves the problem existing in clinical pain treatment, and effectively prevents the side effects, thus further widening the scope of clinical indications to which addictive analgesics are applicable. Imatinib or a derivative thereof in combination with or in the form of various compound preparations (not limited to liquids) with the addictive analgesic agent in a new range of dose ratio (that is, the lowest clinical dose is 100 mg/day, and the highest dose is not more than 400 mg/day) provides a combined drug or preparation of various specifications at a ratio that has a potent analgesic effect, can prevent addiction and tolerance side effect in the treatment of pain with opioids, and is effective for various pains. Moreover, the dose of imatinib mesylate (100-400 mg/day/70 kg) used in the disclosure has few side effects during clinical use, is within a safe dose range for clinical use, has good clinical effectiveness and safety, and can be widely used in the clinical treatment of various acute and chronic pains, to solve the bottleneck problem currently existing in clinical pain treatment.

It is found in the disclosure that a low dose of 5 mg/kg of imatinib mesylate can be used in the treatment and prevention of tolerance, but not in the prevention and treatment of addiction and relapse after withdrawal; and a dose of 10 mg/kg or higher can prevent and treat opioid tolerance and addiction, and can also successfully prevent addiction to opioid and other substances and relapse after withdrawal, without affecting the analgesic effect of morphine. Imatinib mesylate at a clinical dose of 100-400 mg/day/70 kg and at a dose ratio to morphine of less than 2:1 cannot be control nicotine addiction; and when the dose ratio of imatinib mesylate to morphine is 2:1 or greater, opioid addiction and tolerance can be successfully prevented, without affecting the analgesic effect of morphine. This typical dose-effect is similarly exemplified in the clinical use of the second type of antipyretic drug aspirin. For example, a low dose of aspirin is used for antithrombus, a medium dose provides an antipyretic and analgesic effect, and a high dose provides an anti-inflammatory and anti-rheumatic effect. The antithrombotic, antipyretic and analgesic, and anti-inflammatory and anti-rheumatic effects of the drug against clinical indications are completely different and have substantial differences. Therefore, the dose ratio-effect in the disclosure (where dose ratio of imatinib mesylate to opioids in rats is 2:1 or higher, equivalent dose is used for other species, and the clinical dose is 100 mg/day or higher) is a substantial new discovery of imatinib mesylate in the pain treatment and in the prevention of the side effect addiction. Use of a combination or various compound preparations of imatinib mesylate with opioids in this range of dose ratio to prevent and treat the side effect addiction occurring in use of opioid analgesics indicates usefulness of the drug in different dose ranges to different indications, making the disclosure have substantially different applications and advancement over earlier inventions.

Compared with the existing drugs for the treatment of addiction, the disclosure has significantly different mechanism of action. Imatinib mesylate inhibits multiple signaling molecules such as PKC, PI3K-AKT, ERK, and other regulatory kinases, protein expressions, gene expressions, and regulation and initiation of the process of morphine reward, memory and neuroplasticity by blocking the c-kit receptor, thereby blocking the various effects of morphine and achieving the effect of preventing and treating addiction to addictive analgesics.

In the disclosure, imatinib or a derivative thereof and opioids are proposed to be prepared into various compound preparations or combined preparations for preventing addiction side effect for the first time from the perspective of dose ratio and new mechanism of action, thus well meeting the clinical needs, and avoiding unnecessary tragedies caused by abuse. A great advancement is achieved in the safe medication of analgesics on this basis. Imatinib and morphine have no interactive groups in their molecular structures. Therefore, use of imatinib or a derivative thereof in combination with or in the form of a compound preparation with morphine for pain treatment and for the prevention of addiction and tolerance side effects of opioids proposed in the disclosure is feasible, which widens the scope of indications to which opioid analgesics are applicable without affecting the analgesic effect of opioids.

### BRIEF DESCRIPTION OF THE DRAWINGS

FIGS. 1A-1G show new molecular mechanism of imatinib mesylate to prevent morphine addiction.

FIGS. 2A-2D show the dose-effect of imatinib mesylate used with morphine at a certain ratio on the development of addiction in rats, in which FIG. 2A: combined administration of imatinib mesylate with morphine in the prevention of development of CPP, where the dose of morphine for training CPP is 5 mg/kg; FIG. 2B: administration of a compound preparation of imatinib mesylate with morphine in the prevention of development of CPP, where the dose of morphine for training CPP is 5 mg/kg; FIG. 2C: combined administration of imatinib mesylate with morphine in the prevention of development of CPP, where the dose of morphine for training CPP is 10 mg/kg; and FIG. 2D: administration of a compound preparation of imatinib mesylate with morphine in the prevention of development of CPP, where the dose of morphine for training CPP is 10 mg/kg.

FIGS. 3A-3B show a new molecular mechanism of imatinib mesylate to prevent cocaine addiction, in which FIG. 3A: immunohistochemistry shows that imatinib mesylate significantly inhibits the increase of c-Kit in nucleus accumbens, without obvious affecting other brain regions; and FIG. 3B: seven-color immunofluorescence co-labeling shows that after acute alcohol administration, seven key active molecules, including c-Kit, ERK, AKT, and PKC, are co-activated in neurons in nucleus accumbens, and the PDGF activity has no change.

FIGS. 4A-4D show the dose-effect of imatinib mesylate used with cocaine at a certain ratio on the development of addiction in rats, in which FIG. 4A: combined administration of imatinib mesylate with cocaine in the prevention of development of CPP, where the dose of cocaine for training CPP is 5 mg/kg; FIG. 4B: administration of a compound preparation of imatinib mesylate with cocaine in the prevention of development of CPP, where the dose of cocaine for training CPP is 5 mg/kg; FIG. 4C: combined administration of imatinib mesylate with cocaine in the prevention of development of CPP, where the dose of cocaine for training CPP is 10 mg/kg; and FIG. 4D: administration of a compound preparation of imatinib mesylate with cocaine in the prevention of development of CPP, where the dose of cocaine for training CPP is 10 mg/kg.

FIGS. 5A-5B show a new molecular mechanism of imatinib mesylate to prevent ethanol addiction, in which FIG. 5A: immunohistochemistry shows that imatinib mesylate significantly inhibits the increase of c-Kit in nucleus accumbens, without obvious affecting other brain regions; and FIG. 5B: seven-color immunofluorescence co-labeling shows that after acute alcohol administration, seven key active molecules, including c-Kit, ERK, AKT, and PKC, are co-activated in neurons in nucleus accumbens, and the PDGF activity has no change.

FIGS. 6A-6D show the dose-effect of imatinib mesylate used with ethanol at a certain ratio on the development of addiction in rats, in which FIG. 6A: combined administration of imatinib mesylate with ethanol in the prevention of development of CPP, where the dose of ethanol for training CPP is 0.5 g/kg; FIG. 6B: administration of a compound preparation of imatinib mesylate with ethanol in the prevention of development of CPP, where the dose of ethanol for training CPP is 0.5 g/kg; FIG. 6C: combined administration of imatinib mesylate with ethanol in the prevention of development of CPP, where the dose of ethanol for training CPP is 0.75 g/kg; and FIG. 6D: administration of a compound preparation of imatinib mesylate with ethanol in the prevention of development of CPP, where the dose of morphine for training CPP is 0.75 g/kg.

FIGS. 7A-7D show the dose-effect of imatinib mesylate used with morphine at a certain ratio on the drug-seeking behavior after the development of morphine addiction in rats, in which FIG. 7A: inhibition of imatinib mesylate administered in combination with morphine on the drug-seeking behavior, where the dose of morphine for training CPP is 5 mg/kg; FIG. 7B: inhibition of imatinib mesylate administered in the form of a compound preparation with morphine on the drug-seeking behavior, where the dose of morphine for training CPP is 5 mg/kg;

FIG. 7C: inhibition of imatinib mesylate administered in combination with morphine on the drug-seeking behavior, where the dose of morphine for training CPP is 10 mg/kg; and FIG. 7D: inhibition of imatinib mesylate administered in the form of a compound preparation with morphine on the drug-seeking behavior, where the dose of morphine for training CPP is 10 mg/kg.

FIGS. 8A-8D show the dose-effect of imatinib mesylate used with cocaine at a certain ratio on the drug-seeking behavior after the development of cocaine addiction in rats, in which FIG. 8A: inhibition of imatinib mesylate administered in combination with cocaine on the drug-seeking behavior, where the dose of cocaine for training CPP is 5 mg/kg; FIG. 8B: inhibition of imatinib mesylate administered in the form of a compound preparation with cocaine on the drug-seeking behavior, where the dose of cocaine for training CPP is 5 mg/kg; FIG. 8C: inhibition of imatinib mesylate administered in combination with cocaine on the drug-seeking behavior, where the dose of morphine for training CPP is 10 mg/kg; and FIG. 8D: inhibition of imatinib mesylate administered in the form of a compound preparation with cocaine on the drug-seeking behavior, where the dose of cocaine for training CPP is 10 mg/kg.

FIGS. 9A-9D show the dose-effect of imatinib mesylate used with ethanol at a certain ratio on the drug-seeking behavior after the development of ethanol addiction in rats, in which FIG. 9A: inhibition of imatinib mesylate administered in combination with ethanol on the drug-seeking behavior, where the dose of ethanol for training CPP is 0.5 g/kg; FIG. 9B: inhibition of imatinib mesylate administered in the form of a compound preparation with ethanol on the drug-seeking behavior, where the dose of ethanol for training CPP is 0.5 g/kg; FIG. 9C: inhibition of imatinib mesylate administered in combination with ethanol on the drug-seeking behavior, where the dose of ethanol for training CPP is 0.75 g/kg; and FIG. 9D: inhibition of imatinib mesylate administered in the form of a compound preparation with ethanol on the drug-seeking behavior, where the dose of ethanol for training CPP is 0.75 g/kg.

FIGS. 10A-10F show the dose-effect of imatinib mesylate administered with conditioned stimulus or unconditioned stimulus at a certain ratio or directly on the drug-seeking behavior and memory reconsolidation after withdrawal after the development of morphine addiction in rats, where A FIG. 10A: effect of imatinib mesylate administered in combination with unconditioned stimulus, that is, morphine at a dose of 3 mg/kg, on the drug-seeking behavior and relapse, in which the dose of morphine for training CPP is 10 mg/kg; FIG. 10B: effect of imatinib mesylate administered in the form a compound preparation with unconditioned stimulus, that is, morphine at a dose of 3 mg/kg, on the drug-seeking behavior and relapse, in which the dose of morphine for training CPP is 10 mg/kg; FIG. 10C: effect of imatinib mesylate administered in combination with unconditioned stimulus, that is, morphine at a dose of 5 mg/kg, on the drug-seeking behavior and relapse, in which the dose of morphine for training CPP is 10 mg/kg; FIG. 10D: effect of imatinib mesylate administered in the form a compound preparation with unconditioned stimulus, that is, morphine at a dose of 5 mg/kg, on the drug-seeking behavior and relapse, in which the dose of morphine for training CPP is 10 mg/kg; FIG. 10E: effect of imatinib mesylate on the drug-seeking behavior and relapse after induction with conditioned stimulus, in which the dose of morphine for training CPP is 10 mg/kg; and FIG. 10F: effect of imatinib mesylate administered directly on the drug-seeking behavior and relapse, in which the dose of morphine for training CPP is 10 mg/kg.

FIGS. 11A-11F show the dose-effect of imatinib mesylate administered with conditioned stimulus or with unconditioned stimulus at a certain ratio or directly on the drug-seeking behavior and memory reconsolidation after withdrawal after the development of cocaine addiction in rats, where FIG. 11A: effect of imatinib mesylate administered in combination with unconditioned stimulus, that is, cocaine at a dose of 3 mg/kg, on the drug-seeking behavior and relapse, in which the dose of cocaine for training CPP is 10 mg/kg; FIG. 11B: effect of imatinib mesylate administered in the form a compound preparation with unconditioned stimulus, that is, cocaine at a dose of 3 mg/kg, on the drug-seeking behavior and relapse, in which the dose of cocaine for training CPP is 10 mg/kg; FIG. 11A: effect of imatinib mesylate administered in combination with unconditioned stimulus, that is, cocaine at a dose of 5 mg/kg, on the drug-seeking behavior and relapse, in which the dose of cocaine for training CPP is 10 mg/kg; FIG. 11D: effect of imatinib mesylate administered in the form of a compound preparation with unconditioned stimulus, that is, cocaine at a dose of 5 mg/kg, on the drug-seeking behavior and relapse, in which the dose of cocaine for training CPP is 10 mg/kg; FIG. 11E: effect of imatinib mesylate on the drug-seeking behavior and relapse after induction with conditioned stimulus, in which the dose of cocaine for training CPP is 10 mg/kg; and FIG. 11F: effect of imatinib mesylate administered directly on the drug-seeking behavior and relapse, in which the dose of cocaine for training CPP is 10 mg/kg.

FIGS. 12A-12F show the dose-effect of imatinib mesylate administered with conditioned stimulus or unconditioned stimulus at a certain ratio or directly on the drug-seeking behavior and memory reconsolidation after withdrawal after the development of ethanol addiction in rats, where FIG. 12A: effect of imatinib mesylate administered in combination with unconditioned stimulus, that is, ethanol at a dose of 0.25 g/kg, on the drug-seeking behavior and relapse, in which the dose of ethanol for training CPP is 0.75 g/kg; FIG. 12B: effect of imatinib mesylate administered in the form a compound preparation with unconditioned stimulus, that is, ethanol at a dose of 0.25 g/kg, on the drug-seeking behavior and relapse, in which the dose of ethanol for training CPP is 0.75 g/kg; FIG. 12C: effect of imatinib mesylate administered in combination with unconditioned stimulus, that is, ethanol at a dose of 0.5 g/kg, on the drug-seeking behavior and relapse, in which the dose of ethanol for training CPP is 0.75 g/kg; FIG. 12D: effect of imatinib mesylate administered in the form a compound preparation with unconditioned stimulus, that is, ethanol at a dose of 0.5 g/kg, on the drug-seeking behavior and relapse, in which the dose of ethanol for training CPP is 0.75 g/kg; FIG. 12E: effect of imatinib mesylate on the drug-seeking behavior and relapse after induction with conditioned stimulus, in which the dose of ethanol for training CPP is 0.75 g/kg; and FIG. 12F: effect of imatinib mesylate administered directly on the drug-seeking behavior and relapse, in which the dose of morphine for training CPP is 0.75 g/kg.

FIGS. 13A-13D show the effect of imatinib mesylate used with morphine at a certain ratio on the addiction withdrawal reactions in rats, in which FIG. 13A: effect of imatinib mesylate administered in combination with low-dose morphine on withdrawal symptoms (jump times) after the development of morphine-induced CPP; FIG. 13B: effect of imatinib mesylate administered in the form of a compound preparation with low-dose morphine on withdrawal symptoms (jump times) after the development of morphine-induced CPP; FIG. 13C: effect of imatinib mesylate administered in combination with low-dose morphine on withdrawal symptoms (weight loss) after the development of morphine-induced CPP; and FIG. 13D: effect of imatinib mesylate administered in the form of a compound preparation with low-dose morphine on withdrawal symptoms (weight loss) after the development of morphine-induced CPP.

FIGS. 14A-14B show the effect of imatinib mesylate used with cocaine at a certain ratio on the addiction withdrawal reactions in rats, in which FIG. 14A: effect of imatinib mesylate on withdrawal symptoms after the development of cocaine-induced CPP (time spent in the closed arms); and FIG. 14B: effect of imatinib mesylate on withdrawal symptoms after the development of cocaine-induced CPP (closed arm entries).

FIGS. 15A-15B show the effect of imatinib mesylate used with ethanol at a certain ratio on the addiction withdrawal reactions in rats, in which FIG. 15A: effect of imatinib mesylate on withdrawal symptoms after the development of ethanol-induced CPP (time spent in the closed arms); and FIG. 15B: effect of imatinib mesylate on withdrawal symptoms after the development of ethanol-induced CPP (closed arm entries).

FIGS. 16A-16C show the dose-effect of imatinib mesylate used with an opioid addictive substance at a certain ratio on the development and expression of morphine sensitization in rats, in which FIG. 16A: effect of imatinib mesylate administered in combination with morphine on the development of morphine sensitization; FIG. 16B: effect of imatinib mesylate administered in the form of a compound preparation with morphine on the development of morphine sensitization; and FIG. 16C: effect of imatinib mesylate on the expression of morphine sensitization.

FIGS. 17A-17C show the dose-effect of imatinib mesylate used with an opioid addictive substance at a certain ratio on the development and expression of cocaine sensitization in rats, in which FIG. 17A: effect of imatinib mesylate administered in combination with cocaine on the development of cocaine sensitization; FIG. 17B: effect of imatinib mesylate administered in the form of a compound preparation with cocaine on the development of cocaine sensitization; and FIG. 17C: effect of imatinib mesylate on the expression of cocaine sensitization.

FIGS. 18A-18C show the dose-effect of imatinib mesylate used with an ethanol at a certain ratio on the development and expression of ethanol sensitization in rats, in which FIG. 18A: effect of imatinib mesylate administered in combination with ethanol on the development of ethanol sensitization; FIG. 18B: effect of imatinib mesylate administered in the form of a compound preparation with ethanol on the development of ethanol sensitization; and FIG. 18C: effect of imatinib mesylate on the expression of ethanol sensitization.

FIG. 19 shows the dose-effect of imatinib mesylate used with high-sugar and high-fat food at a certain ratio on the addiction to high-sugar and high-fat food in rats, in which the bars from left to right represent high-sugar and high-fat food+physiological saline, high-sugar and high-fat food+imatinib mesylate 1, 5, 10, 20, and 30 mg/kg respectively.

FIGS. 20A-20C show the dose-effect of imatinib mesylate used with high-sugar and high-fat food at a certain ratio on the food-seeking behavior after the development of addiction to high-sugar and high-fat food and the reconsolidation after withdrawal in rats, in which FIG. 20A: effect of imatinib mesylate on the high-sugar and high-fat food seeking behavior and relapse after induction with unconditioned stimulus; FIG. 20B: effect of imatinib mesylate on the high-sugar and high-fat food seeking behavior and relapse after induction with environmental clue; and FIG. 20C: effect of direct administration of imatinib mesylate on the high-sugar and high-fat food seeking behavior and relapse, where the bars from left to right represent high-sugar and high-fat food+physiological saline, high-sugar and high-fat food+imatinib mesylate 1, 5, 10, 20, and 30 mg/kg respectively.

FIGS. 21A-21B show the dose-effect of imatinib mesylate on the gambling task of rats, in which FIG. 21A: effect of imatinib mesylate on the gambling behavior after induction with environmental clue; and FIG. 21B: effect of direct administration of imatinib mesylate on the gambling behavior.

FIG. 22 shows the conversion between the dose of imatinib mesylate and the dose for clinical use.

FIGS. 23A-23D show the effect of imatinib mesylate used with nicotine at a certain dose ratio on the development of nicotine addiction in rats, in which FIG. 23A: effect of imatinib mesylate administered in combination with 0.25 mg/kg of nicotine on the development of nicotine addiction in rats; FIG. 23B: effect of imatinib mesylate administered in combination with 0.5 mg/kg of nicotine on the development of nicotine addiction in rats; FIG. 23C: effect of imatinib mesylate administered in the form of a compound preparation with 0.25 mg/kg of nicotine on the development of nicotine addiction in rats; and FIG. 23D: effect of imatinib mesylate administered in the form of a compound preparation with 0.5 mg/kg of nicotine on the development of nicotine addiction in rats.

FIGS. 24A-24B show a new molecular mechanism of imatinib mesylate in preventing nicotine addiction, in which FIG. 24A: immunohistochemical detection and analysis of c-kit; and FIG. 24B: detection and analysis by Opal/TSA staining of multiple markers.

FIGS. 25A-25D show the effect of imatinib mesylate on the drug-seeking behavior in rats after nicotine addiction after unconditioned stimulus-induced re-arousal of memory, in which FIG. 25A: effect of imatinib mesylate administered in combination with nicotine on the unconditioned stimulus-induced drug-seeking behavior in rats after nicotine addiction, where the dose of nicotine for training CPP is 0.25 mg/kg; FIG. 25B: effect of imatinib mesylate administered in the form of a preparation compound with nicotine on the unconditioned stimulus-induced drug-seeking behavior in rats after nicotine addiction, where the dose of nicotine for training CPP is 0.25 mg/kg; FIG. 25C: effect of imatinib mesylate administered in combination with nicotine on the unconditioned stimulus-induced drug-seeking behavior in rats after nicotine addiction, where the dose of nicotine for training CPP is 0.5 mg/kg; and FIG. 25D: effect of imatinib mesylate administered in the form of a preparation compound with nicotine on the unconditioned stimulus-induced drug-seeking behavior in rats after nicotine addiction, where the dose of nicotine for training CPP is 0.5 mg/kg.

FIGS. 26A-26G show the effect of imatinib mesylate administered after induction with unconditioned stimulus (in combination, or in the form of a compound preparation), after induction with environmental clue, or directly on the drug-seeking behavior and relapse after withdrawal in rats after nicotine addiction, in which FIG. 26A: effect of imatinib mesylate administered in combination with nicotine on the unconditioned stimulus-induced drug-seeking behavior and relapse in rats after nicotine addiction, where the dose of nicotine for training CPP is 0.5 mg/kg, and the dose for induction is 0.1 mg/kg; FIG. 26B: effect of imatinib mesylate administered in the form of a compound preparation with nicotine on the unconditioned stimulus-induced drug-seeking behavior and relapse in rats after nicotine addiction, where the dose of nicotine for training CPP is 0.5 mg/kg, and the dose for induction is 0.1 mg/kg; FIG. 26C: effect of imatinib mesylate administered in combination with nicotine on the unconditioned stimulus-induced drug-seeking behavior and relapse in rats after nicotine addiction, where the dose of nicotine for training CPP is 0.5 mg/kg, and the dose for induction is 0.15 mg/kg; FIG.

FIG. 26D: effect of imatinib mesylate administered in the form of a compound preparation with nicotine on the unconditioned stimulus-induced drug-seeking behavior and relapse in rats after nicotine addiction, where the dose of nicotine for training CPP is 0.5 mg/kg, and the dose for induction is 0.15 mg/kg; FIG. 26E: effect of imatinib mesylate on the environmental clue-induced drug-seeking behavior and relapse in rats after nicotine addiction, where the dose of nicotine for training CPP is 0.5 mg/kg; FIG. 26F: effect of imatinib mesylate administered directly on the drug-seeking behavior and relapse in rats after nicotine addiction, where the dose of nicotine for training CPP is 0.5 mg/kg; and FIG. 26G: imatinib mesylate on the nicotine analogue-induced drug-seeking behavior and relapse in rats after nicotine addiction, where the dose of nicotine for training CPP is 0.5 mg/kg, and the dose for induction is equivalent to 0.15 mg/kg.

FIGS. 27A-27B show the effect of imatinib mesylate used with nicotine at a certain dose ratio on the nicotine withdrawal symptoms, in which FIG. 27A: effect of imatinib mesylate administered in combination with nicotine on the nicotine withdrawal symptoms in rats; and FIG. 27A: effect of imatinib mesylate administered in the form of a compound preparation with nicotine on the nicotine withdrawal symptoms in rats.

FIG. 28 shows the conversion between the dose of imatinib mesylate and the dose for clinical use.

FIG. 29 shows the smoking stopping effect of imatinib mesylate.

FIGS. 30A-30D show the effect of imatinib mesylate used with morphine at a certain dose ratio on the development of morphine addiction in rats, in which FIG. 30A: effect of imatinib mesylate administered in combination with 5 mg/kg of morphine on the development of morphine addiction in rats; FIG. 30B: effect of imatinib mesylate administered in combination with 10 mg/kg of morphine on the development of morphine addiction in rats; FIG. 30C: effect of imatinib mesylate administered in the form of a compound preparation with 5 mg/kg of morphine on the development of morphine addiction in rats; and FIG. 30D: effect of imatinib mesylate administered in the form of a compound preparation with 10 mg/kg of morphine on the development of morphine addiction in rats.

FIGS. 31A-31G show a new molecular mechanism of imatinib mesylate in preventing morphine addiction, in which FIG. 31A: immunohistochemical detection and analysis of c-kit;

FIG. 31B: western-blot of c-kit; FIG. 31C: two-marker immunofluorescence detection and analysis of c-kit and ERK; FIG. 31D: detection and analysis of c-kit and Akt by double-labeling immunofluorescence; FIG. 31E: detection and analysis of c-kit and PKCzeta by double-labeling immunofluorescence; and FIG. 31F: detection and analysis by Opal/TSA staining of multiple markers.

FIGS. 32A-32B show the effect of imatinib mesylate used with morphine at a certain dose ratio on the development of morphine addiction during morphine analgesia, in which FIG. 32A: effect of imatinib mesylate administered in the form of a compound preparation with 10 mg/kg of morphine on the development of morphine addiction in mice; and FIG. 32B: effect of imatinib mesylate administered in the form of a compound preparation with 15 mg/kg of morphine on the development of morphine addiction in mice.

FIGS. 33A-33D show the effect of imatinib mesylate used with morphine at a certain dose ratio on the morphine tolerance in rats, in which FIG. 33A: effect of imatinib mesylate administered in combination with 5 mg/kg of morphine on the morphine tolerance in rats; FIG. 33B: effect of imatinib mesylate administered in combination with 10 mg/kg of morphine on the morphine tolerance in rats; FIG. 33C: effect of imatinib mesylate administered in the form of a compound preparation with 5 mg/kg of morphine on the morphine tolerance in rats; and FIG. 33D: effect of imatinib mesylate administered in the form of a compound preparation with 10 mg/kg of morphine on the morphine tolerance in rats.

FIGS. 34A-34B show the effect of imatinib mesylate used with morphine at a certain dose ratio on the central analgesic effect of morphine in rats, in which FIG. 34A: effect of imatinib mesylate administered in combination with morphine on the central analgesic effect; and FIG. 34B: effect of imatinib mesylate administered in the form of a compound preparation with morphine on the central analgesic effect.

FIGS. 35A-35B show the effect of imatinib mesylate used with morphine at a certain dose ratio on the analgesic effect of morphine in acute visceral pain in mice, in which FIG. 35A: effect of imatinib mesylate administered in combination with morphine on the analgesic effect of morphine in acute visceral pain; and FIG. 35B: effect of imatinib mesylate administered in the form of a compound preparation with morphine on the analgesic effect of morphine in acute visceral pain.

FIGS. 36A-36B show the effect of imatinib mesylate used with morphine at a certain dose ratio on the analgesic effect of morphine in acute inflammatory pain in rats, in which FIG. 36A: effect of imatinib mesylate administered in combination with morphine on the analgesic effect of morphine in acute inflammatory pain; and FIG. 36B: effect of imatinib mesylate administered in the form of a compound preparation with morphine on the analgesic effect of morphine in acute inflammatory pain.

FIGS. 37A-37B show the effect of imatinib mesylate used with morphine at a certain dose ratio on the analgesic effect of morphine in chronic inflammatory pain in rats, in which FIG. 37A: effect of imatinib mesylate administered in combination with morphine on the analgesic effect of morphine in chronic inflammatory pain; and FIG. 36B: effect of imatinib mesylate administered in the form of a compound preparation with morphine on the analgesic effect of morphine in chronic inflammatory pain.

FIG. 38 shows the conversion between the dose of imatinib mesylate and the dose for clinical use.

### DETAILED DESCRIPTION

The disclosure is further described below in detail with reference to examples, but implementations of the disclosure are not limited thereto.

Examples 1 to 18 provide use of a composition of imatinib or a derivative thereof and an addictive substance in the addiction treatment.

The addictive substance used in the following examples is morphine or cocaine. Morphine is widely representative, and those skilled in the art can reproduce similar research results with other addictive substances having similar mechanism of action to morphine. Materials and reagents involved in the examples are commercially available, unless otherwise specified.

### Example 1

Effect of imatinib mesylate on development of morphine addiction in rats and molecular mechanism

### Experiment 1: New Molecular Mechanism of Imatinib Mesylate in Preventing Morphine Addiction

Acute morphine administration.

Animal groups and treatment: The test rats were randomly divided into four groups, including physiological saline+physiological saline group, physiological saline+imatinib mesylate group, morphine+physiological saline group, morphine+imatinib mesylate group (n=10).

Imatinib (30 mg/kg) or normal saline (1 mL/kg) was injected intraperitoneally, morphine (10 mg/kg) was subcutaneously injected half an hour later, and the brain tissue was collected by heart perfusion 1 hr later. The changes of c-kit activity in the mesolimbic dopamine system, including VTA, nucleus accumbens, amygdala, hippocampus, and prefrontal cortex, the changes in phosphorylation level of c-kit were observed by immunohistochemistry combined with western-blot, the distribution of activated cells was observed by immunofluorescence co-labeling and the downstream activated target molecules were determined by multicolor immunofluorescence co-labeling, to determine the new molecular mechanism of morphine addiction and the mechanism of action of imatinib mesylate in controlling addiction.

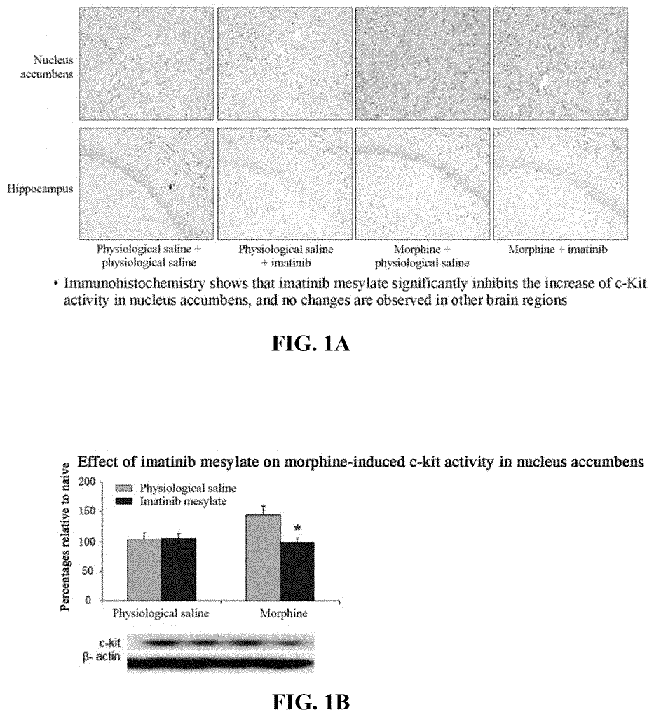

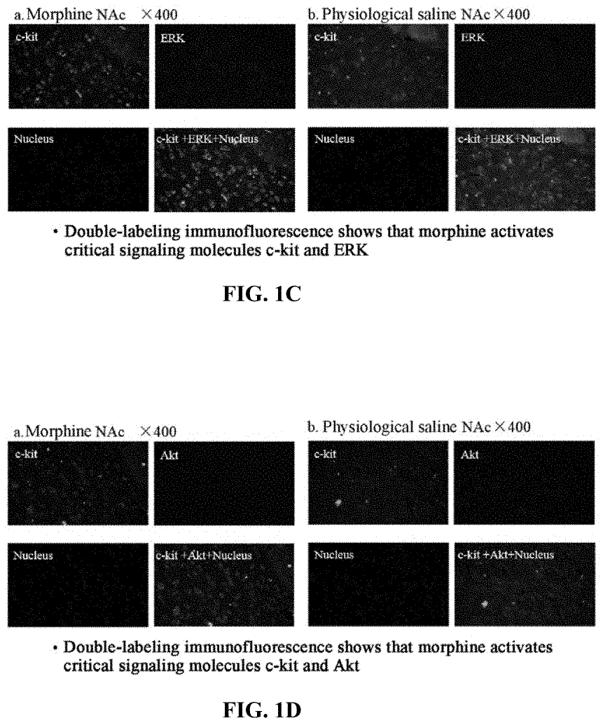

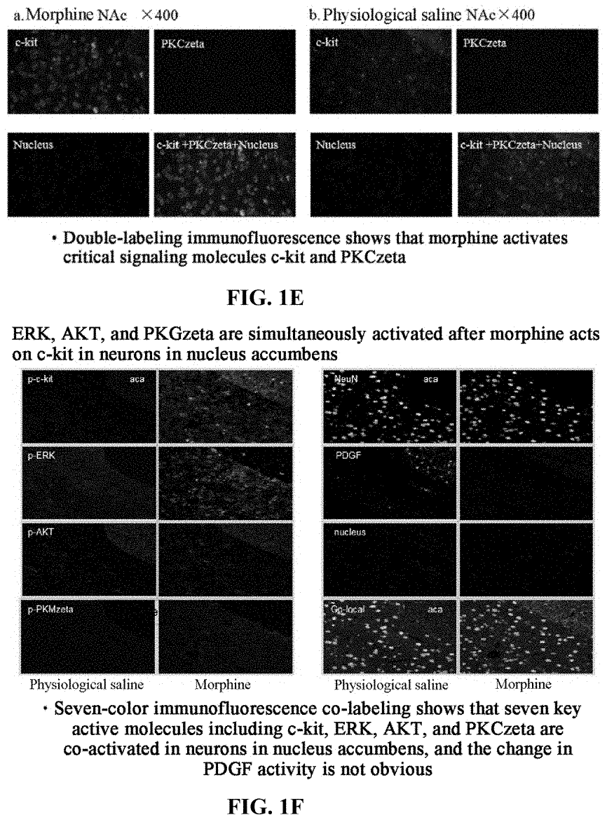

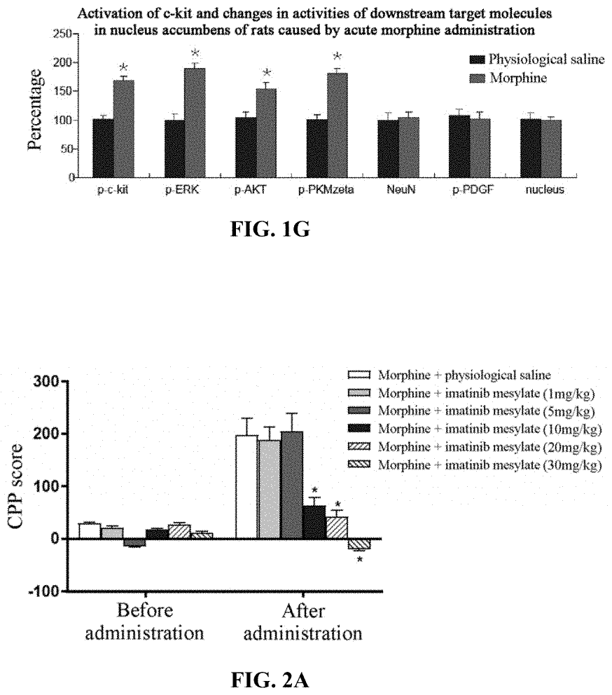

The results are shown in FIGS. 1A-1G. After acute morphine administration, the results of immunohistochemistry, western-blot and multi-color immunofluorescence co-labeling show that the c-kit receptor, and then multiple downstream signaling molecules such as PKC, PI3K-AKT, ERK, and other regulatory kinases, protein expressions, gene expressions, and regulation and initiation of the process of morphine reward, memory and neuroplasticity in neurons in nucleus accumbens instead of in other brain regions are specifically activated. The results suggest that the c-kit receptor in nucleus accumbens is a brain region specifically activated by acute morphine administration. Imatinib mesylate inhibits multiple signaling molecules such as PKC, PI3K-AKT, ERK, and other regulatory kinases, protein expressions, gene expressions, and regulation and initiation of the process of morphine reward, memory and neuroplasticity by blocking the c-kit receptor, to achieve the effect of preventing morphine addiction.

### 2: Effect of Imatinib Mesylate on the Development of Morphine Addiction in Rats

I. Materials

Drug: morphine (Qinghai Pharmaceutical), imatinib mesylate (Selleck Chemicals).

Test animals: SPF-grade male SD rats weighing 180-220 g, provided by Hunan SJA Laboratory Animal Co., Ltd, Animal Certificate No.: 43004700040706, and Production License No.: SCXK (Hunan) 2016-0002. Rat feed: purchased from the Laboratory Animal Center of Wuhan University. All animals were kept in an SPF-grade environment in the Laboratory Animal Center of Wuhan University, at a temperature of 23±2° C. and a humidity of 50±5%, and with light irradiation in a period from 6: 00-18:00 and alternating light and dark period of 12 hrs. Rats were allowed to free access to food and drinking water when they were reared, and habituated for 1 week before the experiment (the same below).

Test instrument: Conditional place preference (CPP) test apparatus (developed by the Institute of Chinese Materia Medica, China Academy of Chinese Medical Sciences). The test was automatically controlled by a computer. The apparatus is a three-compartmented CPP chamber, inducing two side compartments and a middle compartment, where the three compartments are separated by removable partitions and are black internally and externally. The compartments A and B are located at two sides of the middle box and have the same size. 9 yellow light-emitting diodes are provided on a side wall of the compartment A to form a square array, and the bottom plate is formed of stainless steel strips. The bottom plate of the compartment B is formed of a stainless steel mesh. The data regarding time of rats spent in each compartment and the number of entries can be transmitted to the computer, and the behavioral data is automatically collected and recorded.

II. Test Method:

Establishment of morphine-induced CPP model

Baseline test: On day 1, the partitions were removed to open the passages between the three compartments, and the CPP program on the computer was started. the rats were transferred to the middle compartment and allowed to move freely in the three compartments for 15 min. The time spent in each compartment was simultaneously recorded. According to the test results, some rats were excluded, and the remaining rats were grouped, and assigned to a drug-paired side or a nondrug-paired side.

After the baseline test, the rats were divided into 24 groups (n=10) according to their scores:

| Administration in | Administration in the form of |
|---|---|
| combination | a compound preparation |
| Morphine (5 mg/kg) + | Morphine (5 mg/kg) + |
| physiological saline | physiological saline |
| Morphine (5 mg/kg) + | Morphine (5 mg/kg) + |
| imatinib mesylate | imatinib mesylate |
| (1, 5, 10, 20, and 30 mg/kg) | (1, 5, 10, 20, and 30 mg/kg) |
| Morphine (10 mg/kg) + | Morphine (10 mg/kg) + |
| physiological saline | physiological saline |
| Morphine (10 mg/kg) + | Morphine (10 mg/kg) + |
| imatinib mesylate | imatinib mesylate |
| (1, 5, 10, 20, and 30 mg/kg) | (1, 5, 10, 20, and 30 mg/kg) |

CPP training: On days 2 to 9, the passages between the three compartment were closed. On days 2, 4, 6, and 8, rats in the groups receiving administration in combination were injected with different doses of imatinib mesylate (1, 5, 10, 20, and 30 mg/kg, i.p.), and then injected with morphine (5, and 10 mg/kg, s.c.) 30 min later, and transferred to the drug-paired side for 45 min. Rats in the control group were injected with normal saline (1 mL/kg, i.p.)+morphine (5, and 10 mg/kg, s.c.) at the corresponding time points, and transferred to the nondrug-paired side for 45 min. In the group receiving administration in the form of a compound preparation, different doses of imatinib mesylate were mixed with morphine and injected, and then the rats were transferred to the drug-paired side for 45 min. In the control group, physiological saline (1 mL/kg) and morphine (5, and 10 mg/kg) were mixed and injected (s.c.), and transferred to the nondrug-paired side for 45 min. On days 3, 5, 7, and 9, the rats in groups receiving administration in combination and in the control group were all injected with physiological saline (1 mL/kg, i.p.)+physiological saline (1 mL/kg, s.c.). The rats in groups receiving administration in the form of a compound preparation and in the control group were all injected with physiological saline (2 mL/kg, i.p.). The rats in the treatment group were transferred to the nondrug-paired side, the rats in the control group were transferred to the drug-paired side, and the time is 45 min in each case. The drug-paired side for each rat was constant. The rats in each group were returned to the rearing cage after the test.

Test of morphine-induced CPP: On day 10, CPP was tested. Similar to the baseline test, the passages between the three compartments were opened and the rats did not receive any injection. The CPP program on the computer was started. The rats were transferred to the middle compartment, and allowed to move freely in the compartments for 15 min. The time spent in each compartment was simultaneously recorded. CPP score is the difference between the time spent in the drug-paired side and the time spent in the nondrug-paired side. The CPP scores tested before and after training in the CPP test apparatus were compared to determine whether CPP is developed in the rats.

III. Experimental Results

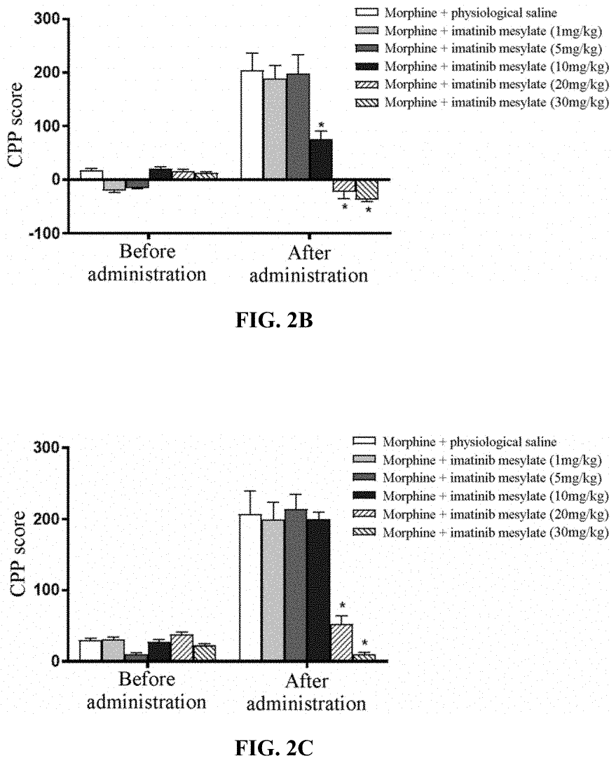

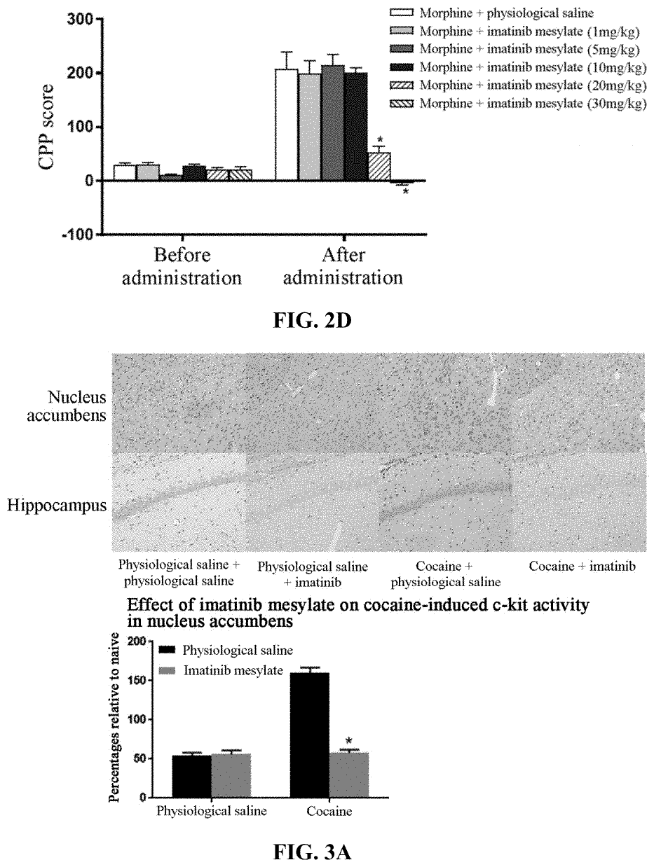

The results are shown in FIGS. 2A-2D, regardless of administration in combination or in the form of a compound preparation, the CPP scores in the treatment groups with imatinib mesylate have significant difference from those in the control groups, and are dose dependent. After intraperitoneal injection of imatinib mesylate (10, 20, and 30 mg/kg i.p.), CPP cannot be developed in rats receiving training of CPP with 5 mg/kg of morphine; in contrast, CPP still exists after injection of imatinib mesylate (1, and 5 mg/kg i.p.). After intraperitoneal injection of imatinib mesylate (20, and 30 mg/kg i.p.), CPP cannot be developed in rats receiving training of CPP with 10 mg/kg of morphine; in contrast, CPP still exists after injection of imatinib mesylate (1, 5, and 10 mg/kg i.p.). CPP still exists in rats without treatment. The results indicate that imatinib mesylate can inhibit the development of morphine addiction in rats only when the dose ratio of imatinib mesylate to morphine for training is 2:1 or greater.

### Example 2

Effect of imatinib mesylate on development of cocaine addiction in rats and molecular mechanism

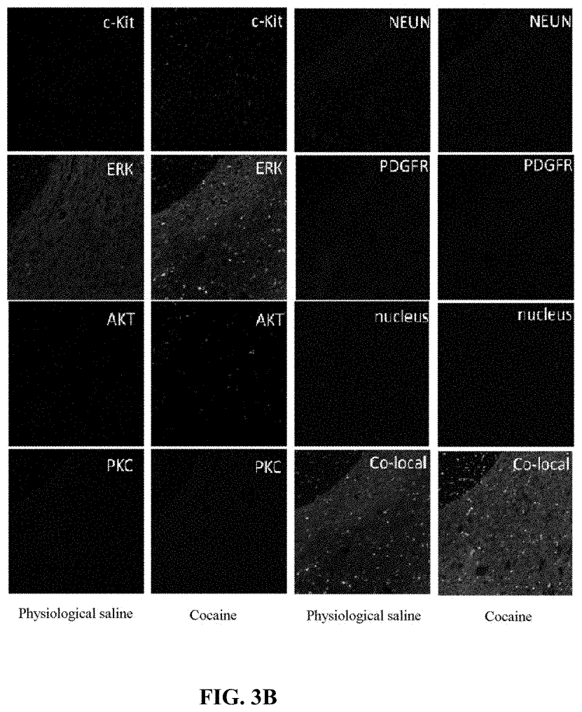

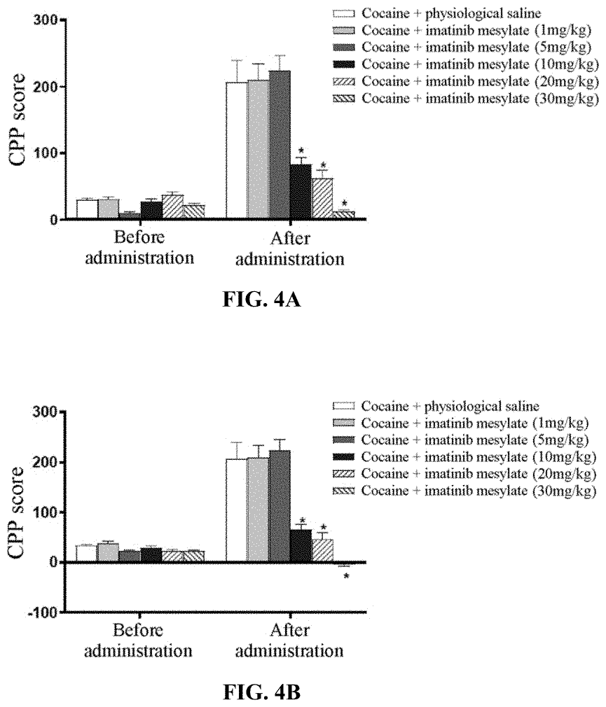

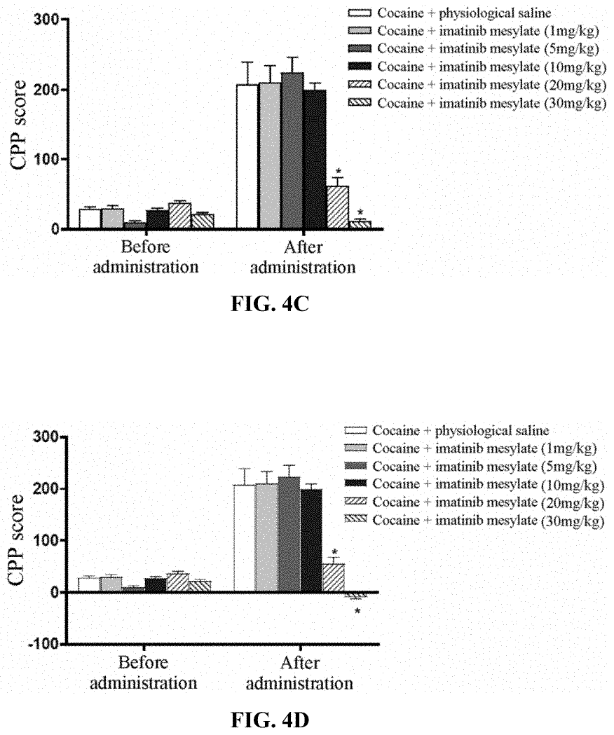

As described in tests 1 and 2 in Example 1, the addiction substance was cocaine in place of morphine, and similar results are obtained. (FIGS. 3A-3B and FIGS. 4A-4D).

### Example 3

Effect of imatinib mesylate on development of ethanol addiction in rats and molecular mechanism

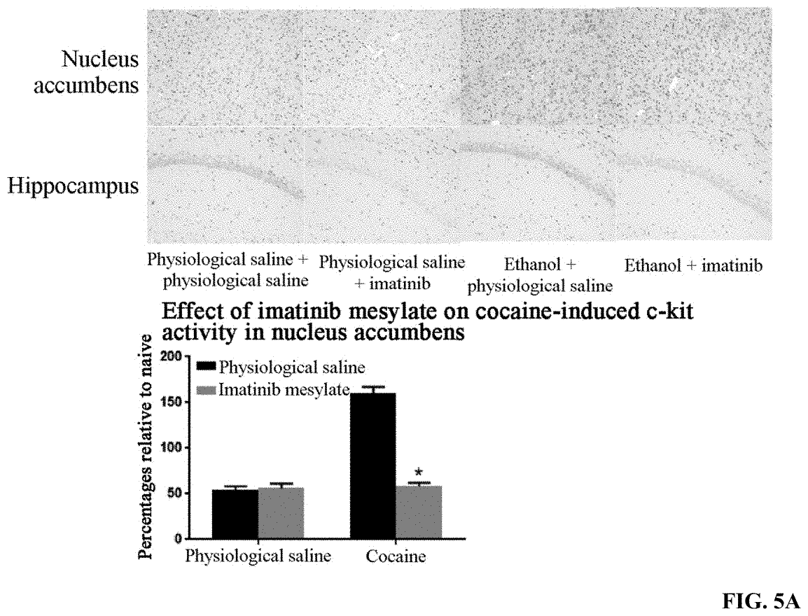

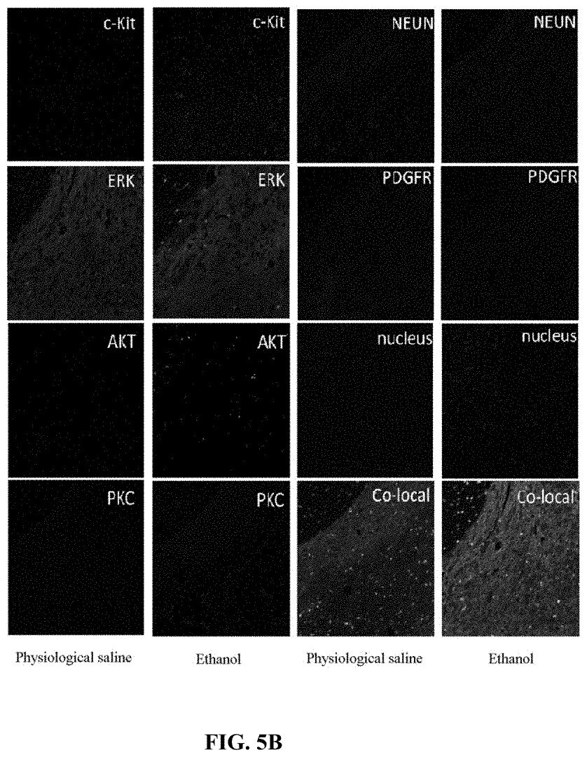

Test 3: The dose of ethanol was 0.75 g/kg, the dose of imatinib mesylate was 30 mg/kg, and the test procedure was the same as that in test 2 in Example 1. Similar results to those in test 2 in Example 1 were obtained. (FIGS. 5A-5B).

Test 4: The dose of ethanol was 0.5, or 0.75 g/kg; the dose of imatinib mesylate is 1, 5, 10, 15, 20, or 30 mg/kg; and the test procedure was the same as that in test 1 in Example 1.

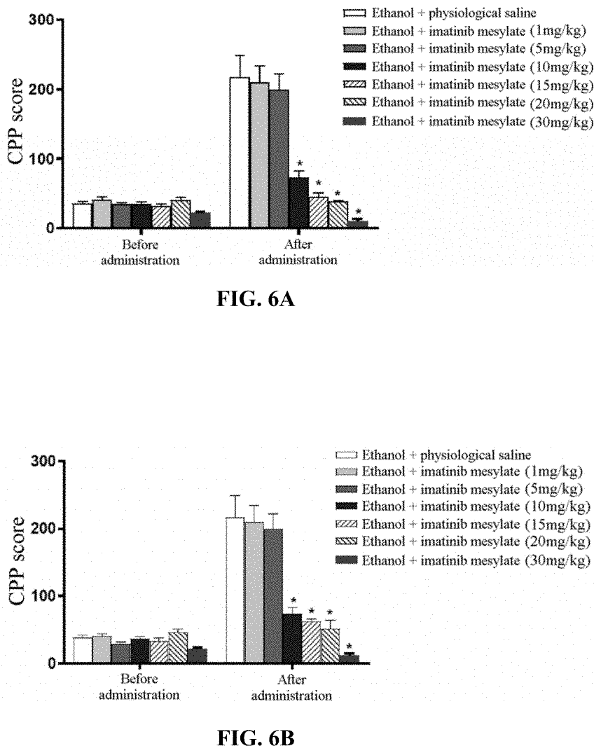

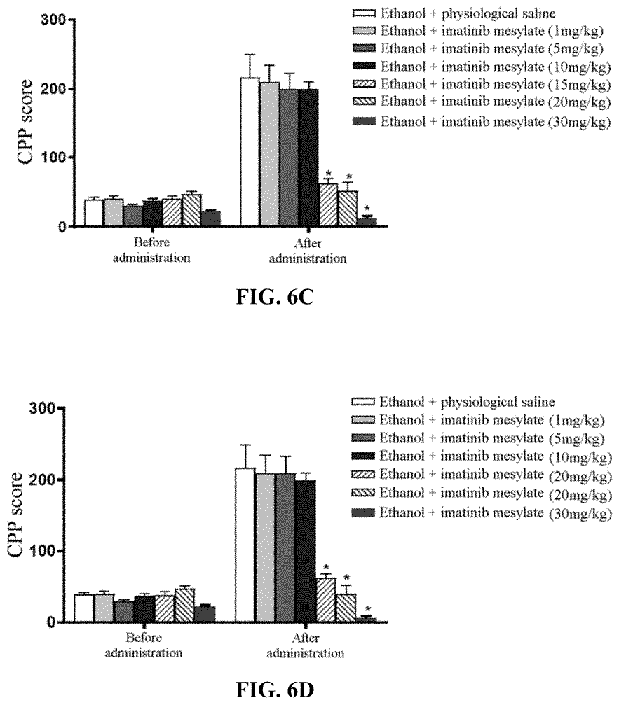

The results are shown in FIGS. 6A-6D. When the dose of ethanol for training CPP is 0.5 g/kg, 1, and 5 mg/kg of imatinib mesylate have no significant effect on the development of CPP; in contrast, 10, 15, 20, and 30 mg/kg of imatinib mesylate can significantly relieve CPP and show a dose dependent effect; and CPP is developed in the group without treatment. When the dose of ethanol for training CPP is 0.75 g/kg, 1, 5, and 10 mg/kg of imatinib mesylate have no significant effect on the development of CPP; in contrast, 15, 20, and 30 mg/kg of imatinib mesylate can significantly relieve CPP and show a dose dependent effect; and CPP is developed in the group without treatment. The results indicate that imatinib mesylate can prevent the development of ethanol addiction when the ratio of dose of imatinib mesylate to the dose of ethanol for training CPP is 1:50 or greater.

### Example 4

Effect of imatinib mesylate on drug-seeking behavior in rats after nicotine addiction after unconditioned stimulus-induced re-arousal of memory

By establishing a morphine-induced CPP model, the effect of various doses (1, 5, 10, 20, and 30 mg/kg; i.p.) of imatinib mesylate on the drug-seeking behavior after morphine addiction was investigated.

I. Material

Drug: morphine (Qinghai Pharmaceutical), imatinib mesylate (Novartis PharmaStein AG).

Test animals: SPF-grade male SD rats weighing 180-220 g, provided by Hunan SJA Laboratory Animal Co., Ltd, Animal Certificate No.: 420110200001490, and Production License No.: SCXK (Hunan) 2017-0067. Rat feed: purchased from the Laboratory Animal Center of Wuhan University.

Test instrument: the same as that in Example 1.

II. Experimental Method:

(1) Establishment of Morphine-Induced CPP Model

Baseline test: On day 1, the passages between three compartments were opened and the CPP program on the computer was started. The rats were transferred to the middle compartment, and allowed to move freely in the three compartments for 15 min. The time spent in each compartment was simultaneously recorded.

After the baseline test, the rats were divided into 12 groups (n=10) according to the CPP score:

| Induction with unconditioned | Induction with unconditioned |
|---|---|
| stimulus (where the dose of | stimulus (where the dose of |
| morphine for training CPP is | morphine for training CPP is |
| 5 mg/kg) | 10 mg/kg) |
| Physiological saline | Physiological saline |
| Imatinib mesylate (1, 5, 10, 20, | Imatinib mesylate (1, 5, 10, 20, |
| and 30 mg/kg) | and 30 mg/kg) |

CPP training: On days 2 to 9, the passages between the three compartment were closed. On days 2, 4, 6, and 8, the rats in the treatment groups were subcutaneously injected with morphine (5, or 10 mg/kg), and then transferred to the drug-paired side for 45 min; and the rats in the control group were subcutaneously injected with physiological saline (1 mL/kg) and then transferred to the nondrug-paired side for 45 min. On days 3, 5, 7, and 9, the rats in the treatment groups and control group were all injected with physiological saline (1 mL/kg). The rats in the treatment group were transferred to the nondrug-paired side, the rats in the control group were transferred to the drug-paired side, and the time is 45 min in each case. The drug-paired side for each rat was constant. The rats in each group were returned to the rearing cage after the test.

Test of morphine-induced CPP: The method was the same as that in Example 1.

(2) Test of Effect of Imatinib Mesylate on Drug-Seeking Behavior

On day 11 of the test, the rats were returned to the drug-paired side for 15 min, and then administered with various doses of imatinib mesylate (1, 5, 10, 20, and 30 mg/kg; i.p.) or physiological saline (1 mL/kg, i.p.).

(3) Retest of Morphine-Induced CPP

On days 1 and 7 after the administration of imatinib mesylate, that is, days 12 and 18 of the test, the rats' preference for the drug-paired compartment within 15 min was tested which was similar to the baseline test.

III. Experimental Results

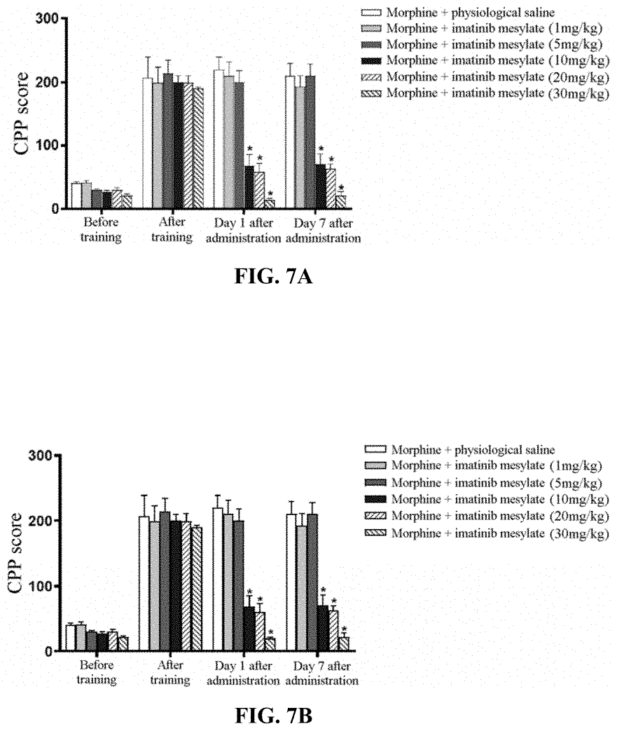

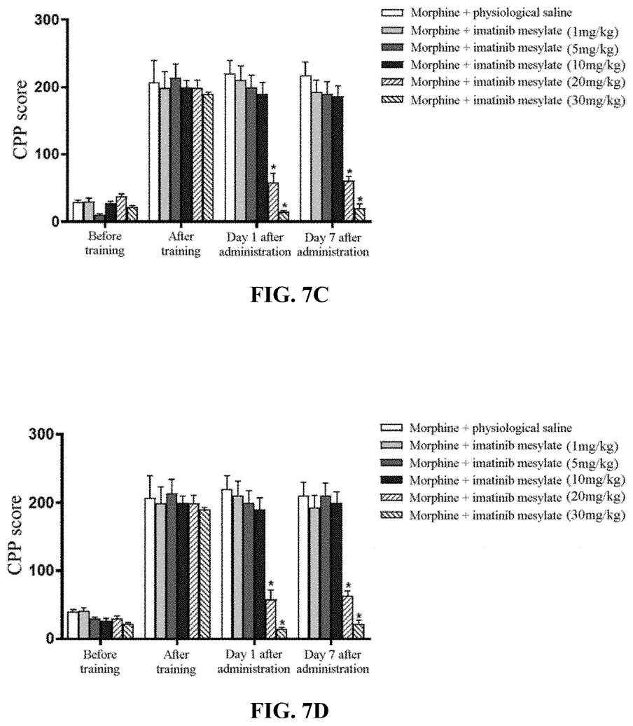

The results are shown in FIGS. 7A-7D. The treatment groups have significant difference from the control groups. For the treatment groups where 5 mg/kg of morphine is used to training CPP, after administration, 1 and 5 mg/kg of imatinib mesylate have no obvious effect; and 10, 20, and 30 mg/kg of imatinib mesylate can significantly relieve CPP and prevent it from being primed in a dose-dependent manner. CPP still exists in rats without treatment. For the groups where 10 mg/kg of morphine is used to training CPP, after administration, 1, 5, and 10 mg/kg of imatinib mesylate have no obvious effect; and 20, and 30 mg/kg of imatinib mesylate can significantly relieve CPP and prevent it from being primed in a dose-dependent manner. CPP still exists in rats without treatment. The results indicate that imatinib mesylate with morphine at a ratio of 2:1 or greater can block the drug-seeking behavior after addiction, suppress the uncontrollable craving, and prevent the relapse.

### Example 5

Effect of imatinib mesylate on drug-seeking behavior in rats after cocaine addiction after unconditioned stimulus-induced re-arousal of memory

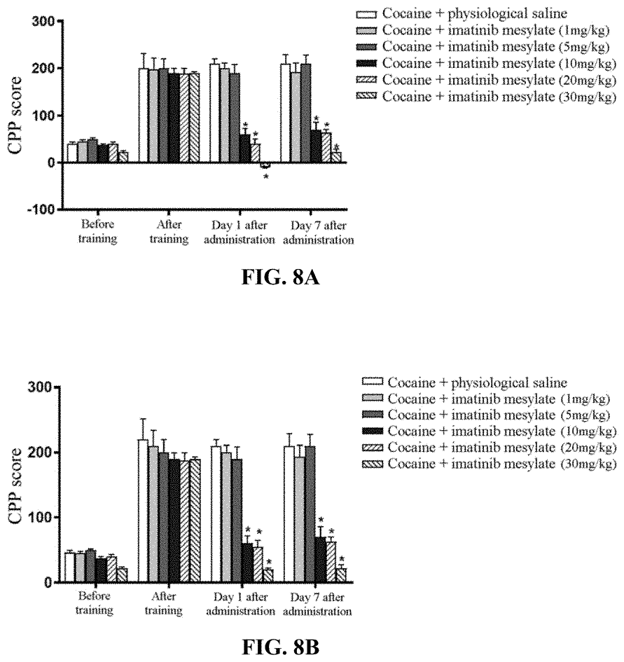

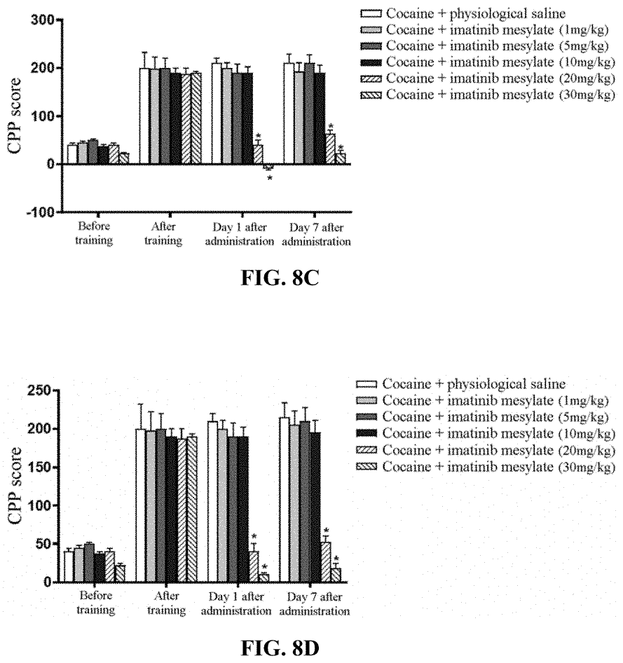

As described in Example 4, the addiction substance was cocaine in place of morphine, and similar results were obtained (FIGS. 8A-8D).

### Example 6

Effect of imatinib mesylate on drug-seeking behavior in rats after ethanol addiction after unconditioned stimulus-induced re-arousal of memory

Groups: A total of 14 groups (n=10) were included.

| Induction with unconditioned | Induction with unconditioned |
|---|---|
| stimulus (where the dose of | stimulus (where the dose of |
| ethanol for training CPP is | ethanol for training CPP is |
| 0.5 g/kg) | 0.75 g/kg) |
| Physiological saline | Physiological saline |
| Imatinib mesylate (1, 5, 10, 15, | Imatinib mesylate (1, 5, 10, 15, |
| 20, and 30 mg/kg) | 20, and 30 mg/kg) |

The rest process was the same as that in Example 3.

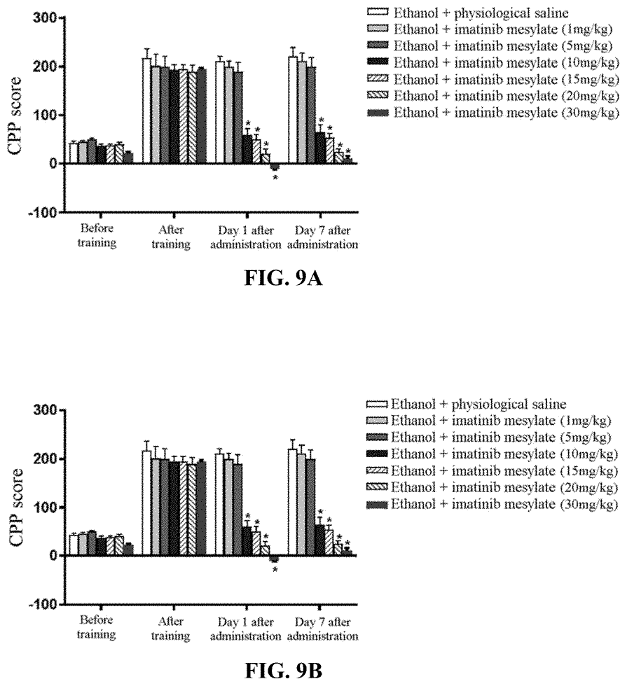

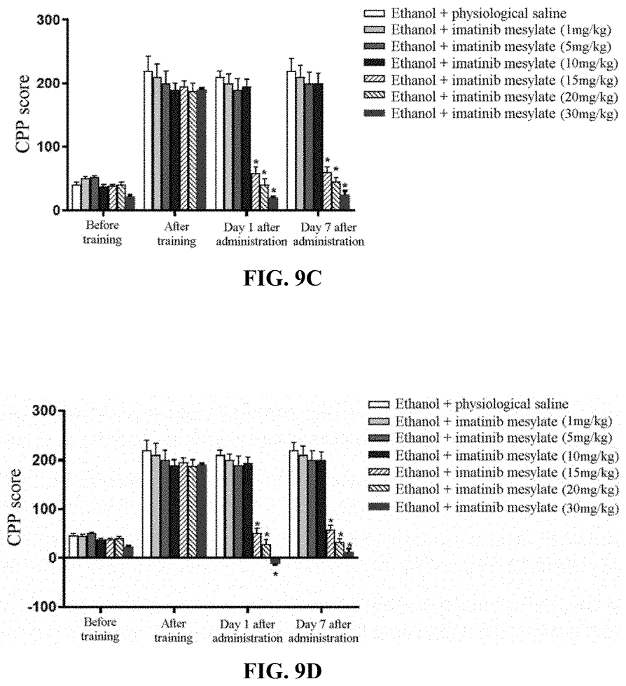

The results are shown in FIGS. 9A-9D. The treatment groups have significant difference from the control groups. For the treatment groups where 0.5 g/kg of ethanol is used to training CPP, after administration, 1, and 5 mg/kg of imatinib mesylate have no obvious effect; and 10, 15, 20, and 30 mg/kg of imatinib mesylate can significantly relieve CPP and prevent it from being primed in a dose-dependent manner. CPP still exists in rats without treatment. For the groups where 0.75 g/kg of ethanol is used to training CPP, after administration, 1, 5, and 10 mg/kg of imatinib mesylate have no obvious effect; and 15, 20, and 30 mg/kg of imatinib mesylate can significantly relieve CPP and prevent it from being primed in a dose-dependent manner. CPP still exists in rats without treatment. The results indicate that imatinib mesylate with ethanol at a ratio of 1:50 or greater can block the drug-seeking behavior after addiction, suppress the uncontrollable craving, and prevent the relapse.

### Example 7

Effect of imatinib mesylate administered after induction with unconditioned stimulus (in combination, or in the form of a compound preparation), after induction with environmental clue, or directly on the drug-seeking behavior after morphine addiction in rats and relapse after withdrawal

By establishing a morphine-induced CPP model, the effect of 30 mg/kg of imatinib mesylate administered directly, after induction with unconditioned stimulus (in combination, or in the form of a compound preparation), and after induction with environmental clue on the drug-seeking behavior after morphine addiction in rats and relapse after withdrawal was explored.

I. Material

Drug: morphine (Qinghai Pharmaceutical), imatinib mesylate (Novartis PharmaStein AG).

Test animals: SPF-grade male SD rats weighing 180-220 g, provided by Hunan SJA Laboratory Animal Co., Ltd, Animal Certificate No.: 420110200001750, Production License No.: SCXK (Hunan) 2017-0012. Rat feed: purchased from the Laboratory Animal Center of Wuhan University.

Test instrument: the same as that in Example 1.

II. Experimental Method:

(1) Establishment of Morphine-Induced CPP Model

Baseline test: On day 1, the passages between three compartments were opened and the CPP program on the computer was started. The rats were transferred to the middle compartment, and allowed to move freely in the three compartments for 15 min. The time spent in each compartment was simultaneously recorded.

After the baseline test, the rats were divided into 24 groups (n=10) according to the CPP score:

| | Induction with | | |
|---|---|---|---|
| | unconditioned | | |
| Induction with | stimulus (ad- | | |
| unconditioned | ministered in | Induction | |
| stimulus (ad- | the form of | with envi- | Direct |
| ministered in | a compound | ronmental | adminis- |
| combination) | preparation) | clue | tration |
| Morphine | Morphine | Physiological | Physiological |
| (induction | (induction | saline | saline |
| with 2, 3, and | with 2, 3, and | | |
| 4 mg/kg) + | 4 mg/kg) + | | |
| physiological | physiological | | |
| saline | saline | | |
| Morphine | Morphine | Imatinib | Imatinib |
| (induction | (induction | mesylate | mesylate |
| with 2, 3, and | with 2, 3, and | (20, and | (20, and |
| 4 mg/kg) + | 4 mg/kg) + | 30 mg/kg) | 30 mg/kg) |
| imatinib | imatinib | | |
| mesylate (20, | mesylate (20, | | |
| and 30 mg/kg) | and 30 mg/kg) | | |

CPP training: On days 2 to 9, the passages between the three compartment were closed. On days 2, 4, 6, and 8, rats in the treatment groups were subcutaneously injected with morphine (10 mg/kg), and then transferred to the drug-paired side for 45 min; and rats in the control group were subcutaneously injected with physiological saline (1 mL/kg) and then transferred to the nondrug-paired side for 45 min. On days 3, 5, 7, and 9, the rats in the treatment groups and control group were all injected with physiological saline (1 mL/kg). The rats in the treatment group were transferred to the nondrug-paired side, the rats in the control group were transferred to the drug-paired side, and the time is 45 min in each case. The drug-paired side for each rat was constant. The rats in each group were returned to the rearing cage after the test.

Test of morphine-induced CPP: The method was the same as that in Example 1.

(2) Test of Effect of Imatinib Mesylate on Drug-Seeking Behavior

On day 11 of the test, for induction with substance clue, the rats in the group receiving administration in combination were injected with morphine (2, 3, or 4 mg/kg; s.c.) or physiological saline (1 mL/kg, s.c.), and then intraperitoneally administered with 30 mg/kg of imatinib mesylate 30 min later; and the rats in the group receiving administration in the form of a compound preparation were administered with a mixture of 30 mg/kg of imatinib mesylate with morphine (2, 3, or 4 mg/kg, i.p.) or physiological saline (1 mL/kg). The rats for induction with environmental clue were returned to the drug-paired side for 15 min, and then intraperitoneally injected with imatinib mesylate of 30 mg/kg or physiological saline of 1 mL/kg. The rats without induction were directly administered with imatinib mesylate (30 mg/kg, i.p.) or physiological saline (1 mL/kg, i.p.). The rats were then returned to the rearing cage, and tested for CPP after 24 hrs.

(3) Retest of Morphine-Induced CPP

On days 1 and 7 after the administration of imatinib mesylate, that is, days 12 and 18 of the test, the rats' preference for the drug-paired compartment within 15 min was tested, which was similar to the baseline test. On day 19, the rats were primed with a small dose of morphine (3 mg/kg, s.c.). After morphine injection, the rats were transferred to the middle compartment, and subjected to CPP tests for 15 min. The rats did not receive any treatment during the non-test period.

III. Experimental Results

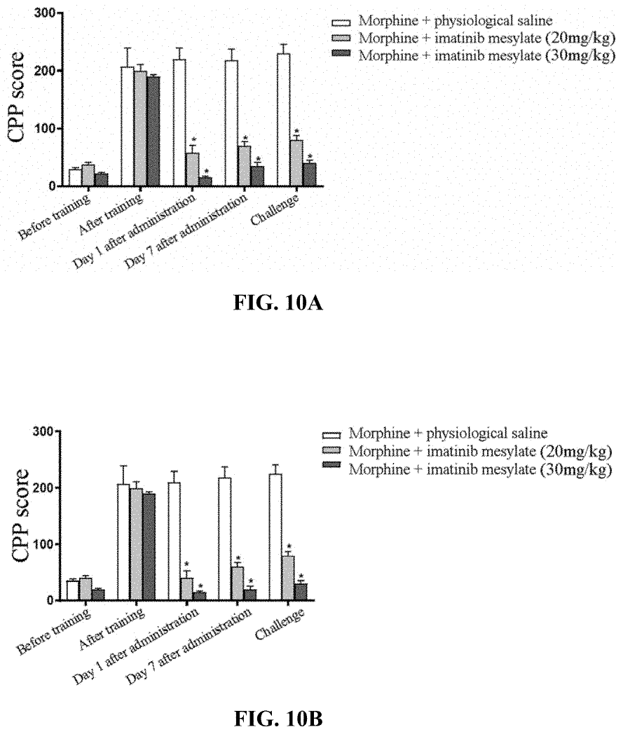

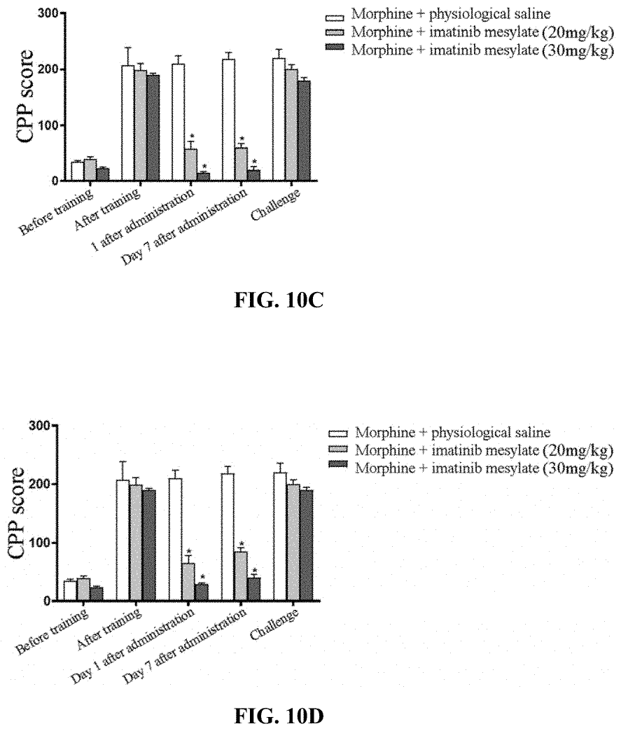

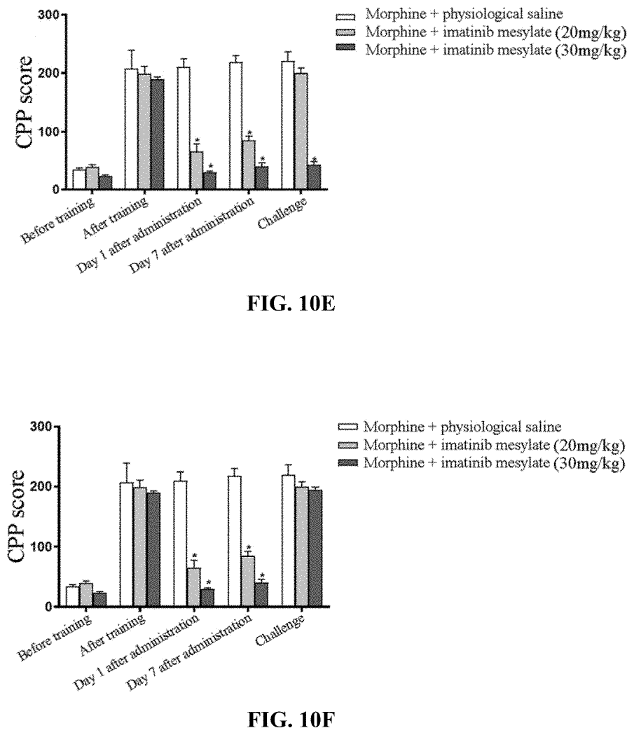

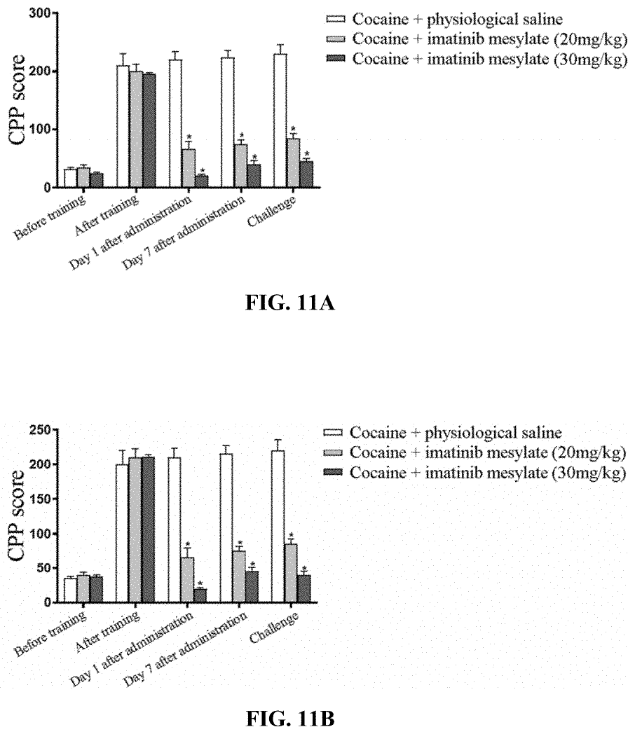

The results are shown in FIGS. 10A-10F. The treatment groups have significant difference from the control groups. The administration of 10, 20, and 30 mg/kg of imatinib mesylate can significantly reduce the CPP in all treatment groups. CPP still exists in rats without treatment. The results indicate that imatinib mesylate administered with morphine at a ratio of 2:1 can block the drug-seeking behavior after addiction, suppress the uncontrollable craving. After challenge with a small dose of morphine, for induction with unconditioned stimulus both in the group receiving administration in combination and in the group receiving administration in the form of a compound preparation, 10, 20, and 30 mg/kg of imatinib mesylate prevent the rats from being primed, when the dose of morphine for induction is 3 mg/kg (including administration in combination and in the form of a compound preparation). For induction with environmental clue, only the rats in the groups with 20 and 30 mg/kg of imatinib mesylate are not primed. The rats in the group receiving direct administration of imatinib mesylate are all primed. The results indicate that relapse can be prevented when administered after induction with unconditioned stimulus (in combination and in the form of a compound preparation). All the doses can inhibit the drug-seeking behavior, prevent the rats from being primed, and well control the relapse only when the ratio of dose of imatinib mesylate used to the dose of morphine for training is 2:1 or greater, and the ratio of dose of morphine for unconditioned stimulus to the dose of morphine for training is 1:3 or less. When administered after induction with environmental clue (in combination and in the form of a compound preparation), the drug-seeking behavior can only be partially prevented, and the effect of preventing relapse is weak. The direct administration of imatinib mesylate can block the drug-seeking behavior only when the dose of imatinib mesylate is as high as 30 mg/kg, and the effect of preventing relapse is extremely weak.

### Example 8

Effect of imatinib mesylate administered after induction with unconditioned stimulus (in combination, or in the form of a compound preparation), after induction with environmental clue, or directly on the drug-seeking behavior after cocaine addiction and relapse after withdrawal in rats

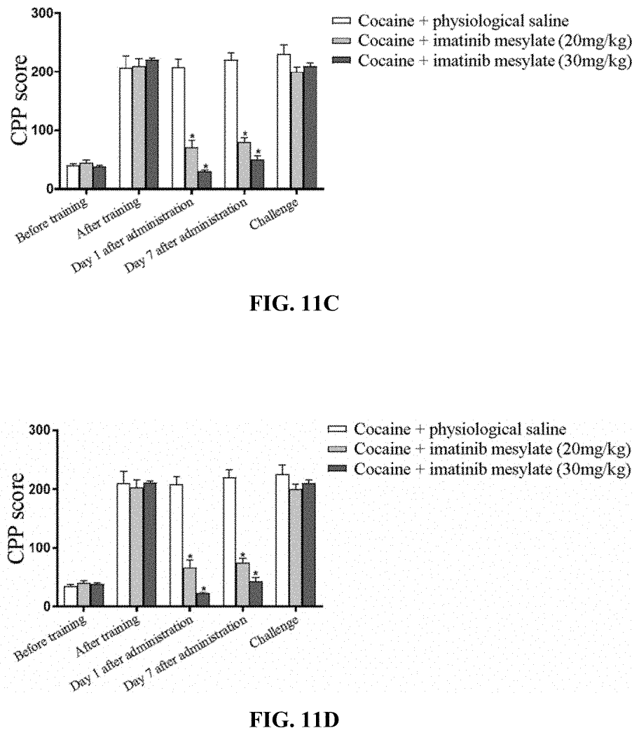

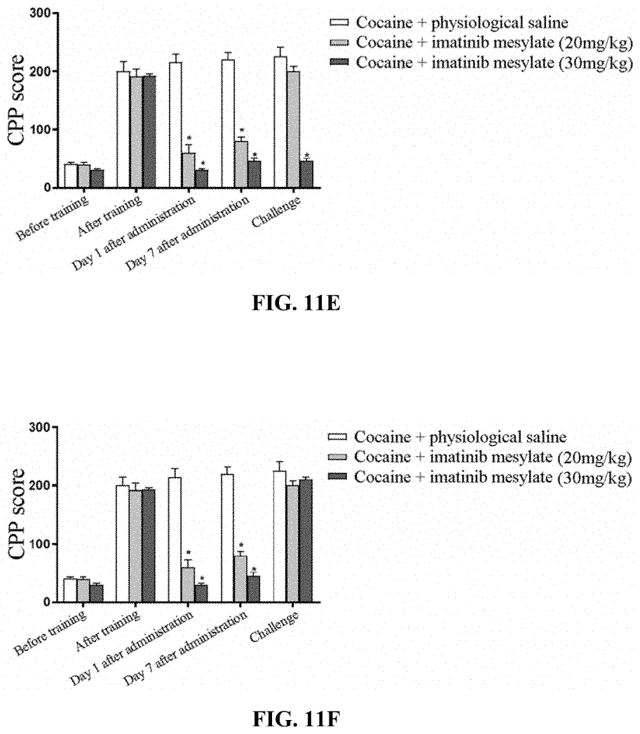

As described in Example 7, the addiction substance was cocaine in place of morphine, and similar results were obtained (FIGS. 11A-11F).

### Example 9

Effect of imatinib mesylate administered after induction with unconditioned stimulus (in combination, or in the form of a compound preparation), after induction with environmental clue, or directly on the drug-seeking behavior after ethanol addiction and relapse after withdrawal in rats

Groups:

| | Induction with | | |
|---|---|---|---|
| Induction with | substance clue | | |
| substance | (administration | Induction | |
| clue (adminis- | in the form | with envi- | Direct |
| tration in | of a compound | ronmental | adminis- |
| combination) | preparation) | clue | tration |
| Ethanol (0.15, | Ethanol (0.15, | Physiological | Physiological |
| 0.25, and 0.5 | 0.25, and 0.5 | saline | saline |
| g/kg) + | g/kg) + | | |
| physiological | physiological | | |
| saline | saline | | |
| Ethanol (0.15, | Ethanol (0.15, | Imatinib | Imatinib |
| 0.25, and 0.5 | 0.25, and 0.5 | mesylate | mesylate |
| g/kg) + | g/kg) + | (15, and | (15, and |
| imatinib | imatinib | 30 mg/kg) | 30 mg/kg) |
| mesylate (15, | mesylate (15, | | |
| and 30 mg/kg) | and 30 mg/kg) | | |

The dose of ethanol for training CPP is 0.75 g/kg, and the dose of ethanol for challenge on day 19 is 0.3 g/kg. The rest process was the same as that in Example 5.

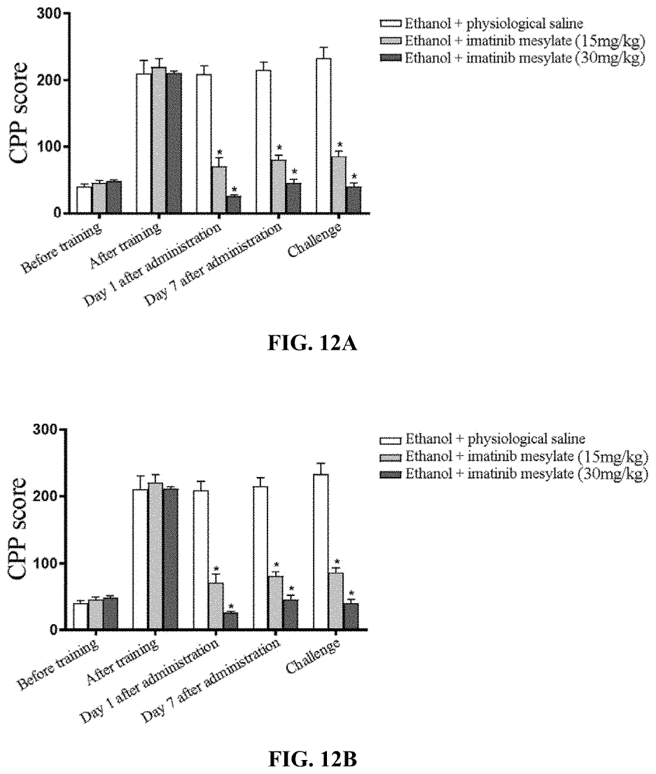

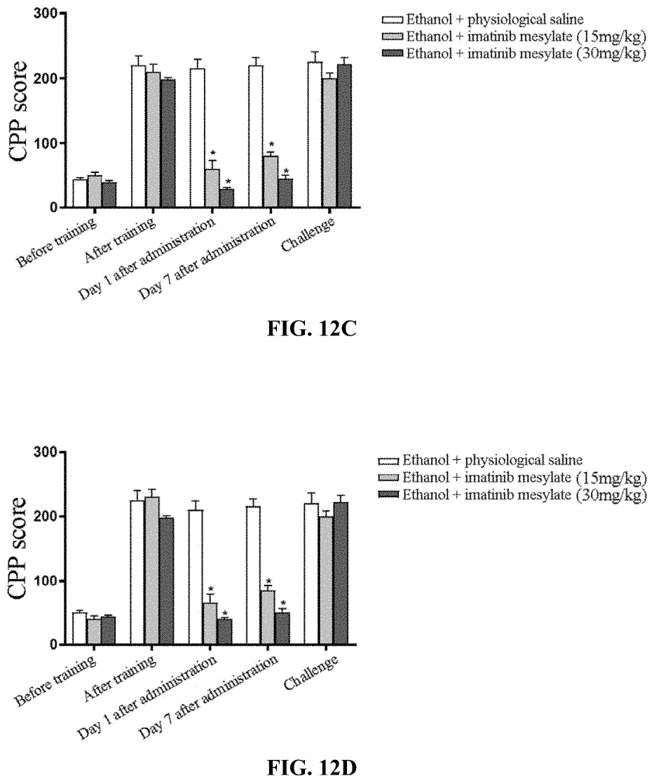

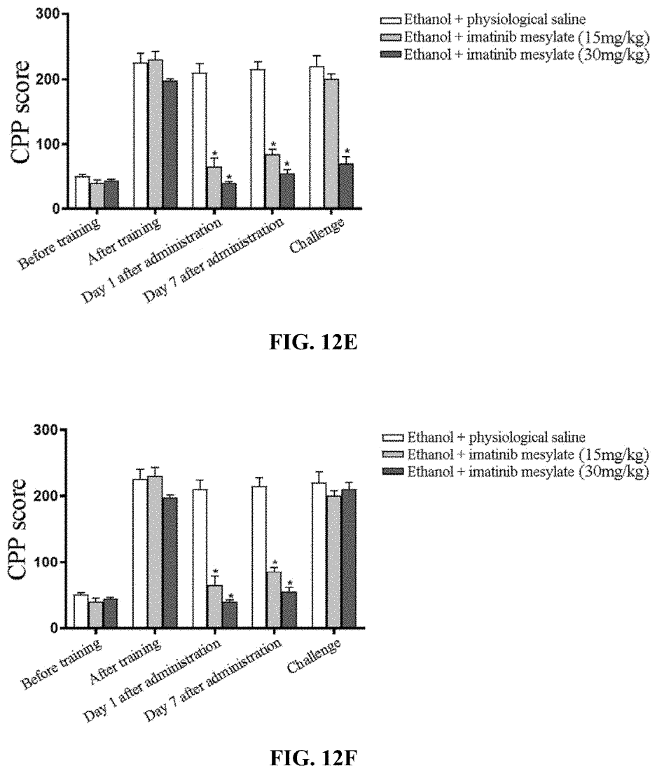

The results are shown in FIGS. 12A-12F. The treatment groups have significant difference from the control groups. Administration of 15, and 30 mg/kg of imatinib mesylate can significantly reduce the CPP in all treatment groups. CPP still exists in rats without treatment. The results indicate that imatinib mesylate administered with ethanol at a ratio of 1:50 can block the drug-seeking behavior after addiction, and suppress the uncontrollable craving. After challenge with a small dose of ethanol, for induction with substance clue both in the group receiving administration in combination and in the group receiving administration in the form of a compound preparation, the rats in groups receiving 15, and 30 mg/kg of imatinib mesylate are not primed, when the dose of ethanol for induction is 0.25 g/kg (including administration in combination and in the form of a compound preparation). For induction with environmental clue, only the rats in the group with 30 mg/kg of imatinib mesylate are not primed. The rats in the group receiving direct administration of imatinib mesylate are all primed. The results indicate that relapse can be prevented when administered after induction with unconditioned stimulus (in combination and in the form of a compound preparation). Imatinib mesylate has this effect only when the ratio of dose of imatinib mesylate used to the dose of ethanol for training is 1:50 or greater, and the ratio of dose of ethanol for unconditioned stimulus to the dose of ethanol for training is 1:3 or less. When administered after induction with environmental clue (in combination and in the form of a compound preparation), only the drug-seeking behavior, but not the relapse, can be prevented. The direct administration of imatinib mesylate can prevent the drug-seeking behavior only when the dose of imatinib mesylate is as high as 30 mg/kg, and cannot prevent relapse.

### Example 10

Effect of imatinib mesylate on withdrawal symptoms in rats after development of morphine-induced CPP

I. Material

Drug: morphine (Qinghai Pharmaceutical), imatinib mesylate (Selleck Chemicals).

Test animals: SPF-grade male SD mice weighing 32-36 g, provided by Hunan SJA Laboratory Animal Co., Ltd, Animal Certificate No.: 42010200001574, Production License No.: SCXK (Hunan) 2016-0002. Rat feed: purchased from the Laboratory Animal Center of Wuhan University.

Test instrument: 5 L transparent beaker, and stopwatch

II. Experimental Method:

(1) Training of Morphine-Induced CPP

The baseline was tested.

Animal groups and treatments: After the baseline was tested, the mice were divided into 12 groups (n=10), including:

| | Administration in the form of |
|---|---|
| Administration in combination | a compound preparation |
| Morphine + physiological saline | Morphine + physiological saline |
| Morphine + imatinib mesylate | Morphine + imatinib mesylate |
| (1.5, 7.5, 15, 30, and 45 mg/kg) | (1.5, 7.5, 15, 30, and 45 mg/kg) |

The rest process was similar to that in Example 5.

(2) Observation of Withdrawal Symptoms

On the day of the test, the mice in the group receiving administration in combination were given different doses of imatinib mesylate (1.5, 7.5, 15, 30, and 45 mg/kg, i.p.). The mice in the control group were injected with physiological saline (1 mL/kg, i.p.). After 0.5 hr, all mice were injected with low-dose morphine (5 mg/kg, s.c.), and then with naloxone (2 mg/kg, s.c.) after 1 hr, and the withdrawal symptoms were observed for half an hour, where the indices observed were jump times and changes in body weight. The mice in the group receiving administration in the form of a compound preparation were subcutaneously administered with a mixed agent of different doses of imatinib mesylate (1.5, 7.5, 15, 30, and 45 mg/kg) or physiological saline (1 mL/kg) with morphine (5 mg/kg), and then with naloxone (2 mg/kg, s.c.) after 1 hr, and the withdrawal symptoms were observed for half an hour, where the indicators observed were jump times and changes in body weight.

III. Experimental Results

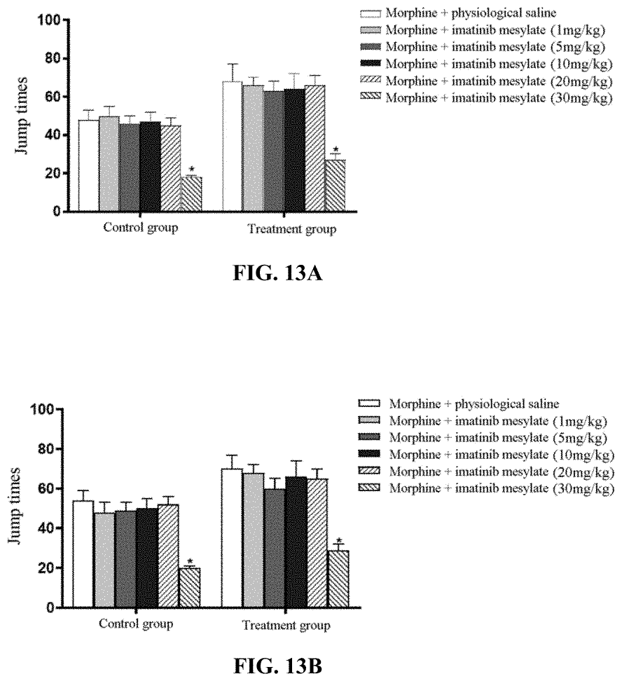

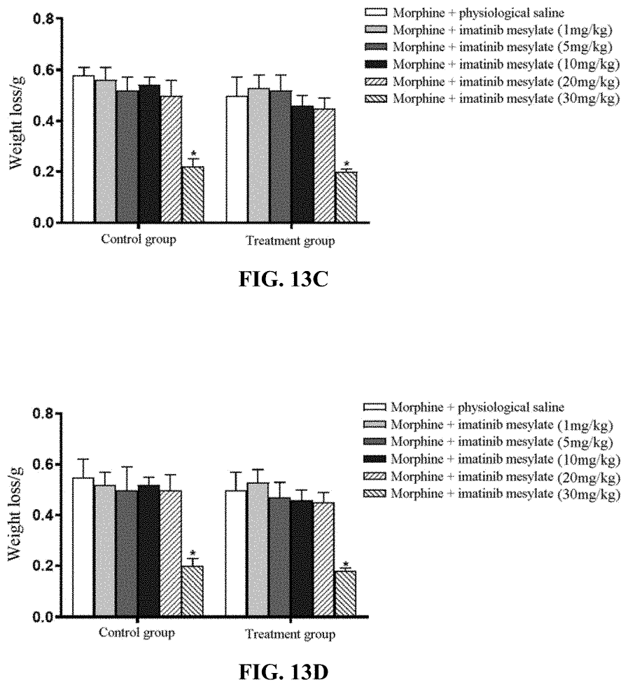

The results are shown in FIGS. 13A-13D. Compared with the control group, regardless of administration in combination or in the form of a compound preparation, 15, 30, and 45 mg/kg of imatinib mesylate can alleviate the withdrawal symptoms in mice.

### Example 11

Effect of imatinib mesylate on withdrawal symptoms in rats after development of cocaine-induced CPP

I. Material

Drug: cocaine (Qinghai Pharmaceutical), imatinib mesylate (Selleck Chemicals).

Test instruments: CPP test apparatus, and elevated plus maze

II. Experimental Method:

(1) Training of Cocaine-Induced CPP

The baseline was tested. After the baseline test, the rats were divided into 12 groups (n=10), including:

| | Administration in the form of |
|---|---|
| Administration in combination | a compound preparation |
| Cocaine + physiological saline | Cocaine + physiological saline |
| Cocaine + imatinib mesylate | Cocaine + imatinib mesylate |
| (1.5, 7.5, 15, 30, and 45 mg/kg) | (1.5, 7.5, 15, 30, and 45 mg/kg) |

The dose of cocaine for training is 10 mg/kg, and the process was the same as the process for training cocaine-induced CPP in Example 5.

(2) Elevated Plus Maze

After the development of CPP, the drug was withdrawn for 72 hrs, and then the elevated plus maze test was performed.

The mice were intraperitoneally injected with 1.5, 7.5, 15, 30, or 45 mg/kg of imatinib mesylate or 1 mL/kg of physiological saline, and then transferred to the center of the elevated maze after 1 hr. The entries of the mice into and the time spent in closed arm was counted.

III. Experimental Results

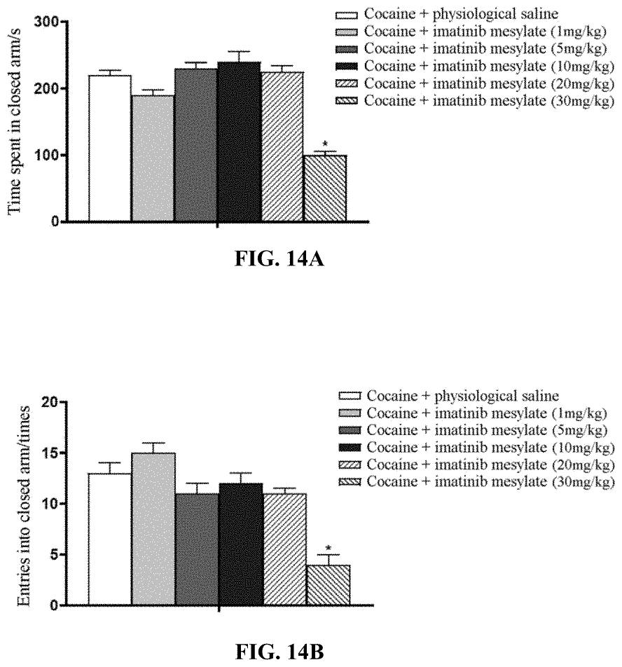

The results are shown in FIGS. 14A-14B. Compared with the control group, the treatment groups have obvious difference. The time spent in the closed arm and the entries into the closed arm of rats dosed with 30 mg/kg are significantly lower than those in the other groups, indicating that the withdrawal symptoms after cocaine addiction can be inhibited when the ratio of dose of imatinib mesylate to the dose of cocaine for training cocaine-induced CPP is 2:1 or greater.

### Example 12

Effect of imatinib mesylate on withdrawal symptoms in rats after development of ethanol-induced CPP

I. Test instruments: CPP test apparatus, and elevated plus maze

II. Experimental Method:

(1) Development of Ethanol-Induced CPP

The baseline was tested. After the baseline test, the rats were divided into 14 groups, including

| | Administration in the form of |
|---|---|
| Administration in combination | a compound preparation |
| Ethanol + physiological saline | Ethanol + physiological saline |
| Ethanol + imatinib mesylate | Ethanol + imatinib mesylate |
| (1.5, 7.5, 15, 30, and 45 mg/kg) | (1.5, 7.5, 15, 30, and 45 mg/kg) |

The dose of ethanol for developing CPP was 0.75 g/kg. The rest process was the same as that in Example 6.

(2) Elevated Plus Maze

After the development of CPP, the drug was withdrawn for 72 hrs, and then the elevated plus maze test was performed.

The mice were intraperitoneally injected with 1.5, 7.5, 15, 30, or 45 mg/kg of imatinib mesylate or 1 mL/kg of physiological saline, and then transferred to the center of the elevated maze after 1 hr. The entries of the mice into and the time spent in closed arm was counted.

III. Experimental Results

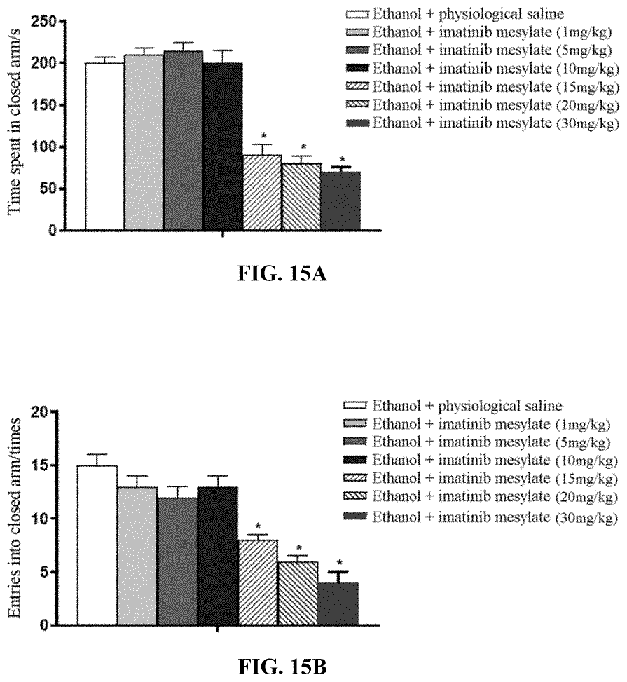

The results are shown in FIGS. 15A-15B. Compared with the control group, regardless of administration in combination or in the form of a compound preparation, 15, 30, and 45 mg/kg of imatinib mesylate can alleviate the withdrawal symptoms in mice.

### Example 13

Dose-effect of imatinib mesylate on development and expression of morphine sensitization in rats

According to the results of Examples 1, 4 and 7, imatinib mesylate used with opioids at a certain dose ratio can inhibit opioid addiction and relapse after withdrawal in rats. In this test, a morphine sensitization model in rats was established to explore the improvement of imatinib mesylate with opioid used at a certain ratio on morphine-induced spontaneous hyperactivity in rats, so as to determine a low-toxicity drug having clear efficacy against spontaneous hyperactivity induced after morphine addiction.

I. Material

Drug: morphine (Qinghai Pharmaceutical), imatinib mesylate (Selleck Chemicals).

Test animals: SPF-grade male SD rats weighing 180-220 g, provided by Hunan SJA Laboratory Animal Co., Ltd, Animal Certificate No.: 4201200001721, Production License No.: SCXK (Hunan) 2017-0012. Rat feed: purchased from the Laboratory Animal Center of Wuhan University.

Test instrument: spontaneous activity detecting apparatus (developed by the Institute of Chinese Materia Medica, China Academy of Chinese Medical Sciences). The test was automatically controlled by a computer. The apparatus includes four spontaneous activity observation chamber, a video synthesizer, a video pattern sampling card, and analysis software. The system performs video tracking of the rat activity and automatically records the rat activity track and activity times. The spontaneous activity evaluation index is the total activity times in a certain period of time (such as 60 min), and an increase in the total activity times indicates an increase in spontaneous activity.

II. Experimental Method:

Establishment of animal model: 1 day before the test (day 0), Baseline activity of the test rats was measured, and the rats were randomly divided into 14 groups (n=10) according to the measurement results.

| Effect on development | Effect on expression |
|---|---|
| Physiological saline + | Physiological saline + |
| physiological saline | physiological saline |
| Morphine + physiological saline | Morphine + physiological saline |
| Morphine + imatinib mesylate | Morphine + imatinib mesylate |
| (1, 5, 10, 20, and 30 mg/kg) | (1, 5, 10, 20, and 30 mg/kg) |

Effect on development: On days 1 to 5 of the test, physiological saline (1 mL/kg) or imatinib mesylate (1, 5, 10, 20, and 30 mg/kg) were intraperitoneally administered, and then morphine (10 mg/kg) was subcutaneously injected 30 min later; or a mixture of imatinib mesylate or physiological saline with morphine was subcutaneously administered, for 5 consecutive days.

Effect on expression: On days 1 to 5 of the test, morphine (10 mg/kg) or physiological saline (1 mL/kg) were subcutaneously injected, for 5 consecutive days. Then the drug was withdrawn for 5 days. On day 11 of the test, the rats were injected with imatinib mesylate (1, 5, 10, 20, and 30 mg/kg, i.p.) or physiological saline (1 mL/kg, i.p.), and then challenged by subcutaneously injecting small-dose morphine (5 mg/kg). The spontaneous activity behavior in each group was detected for 60 minutes, and the changes in the spontaneous activity behavior were observed.

III. Experimental Results

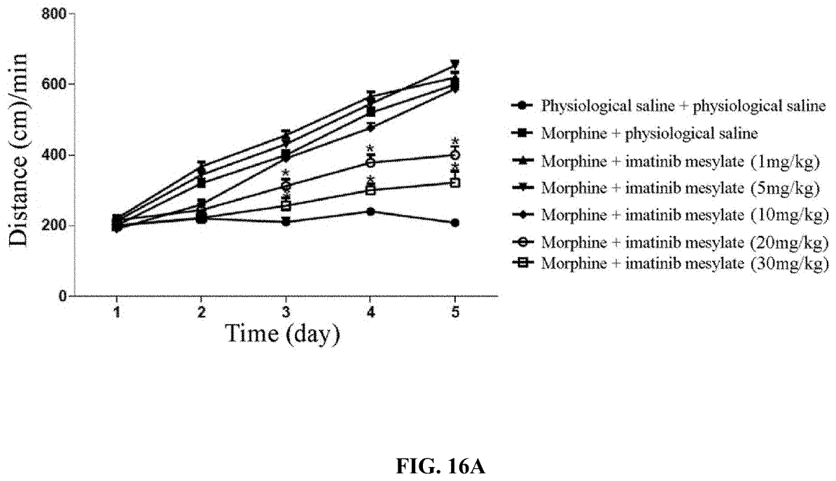

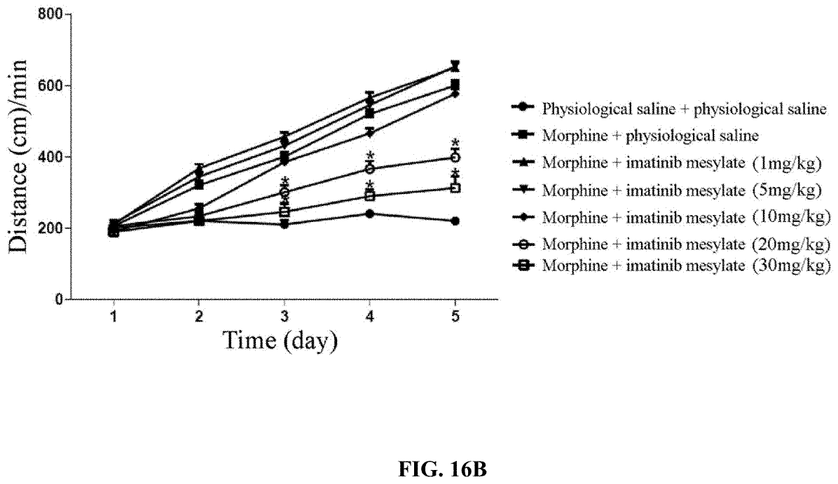

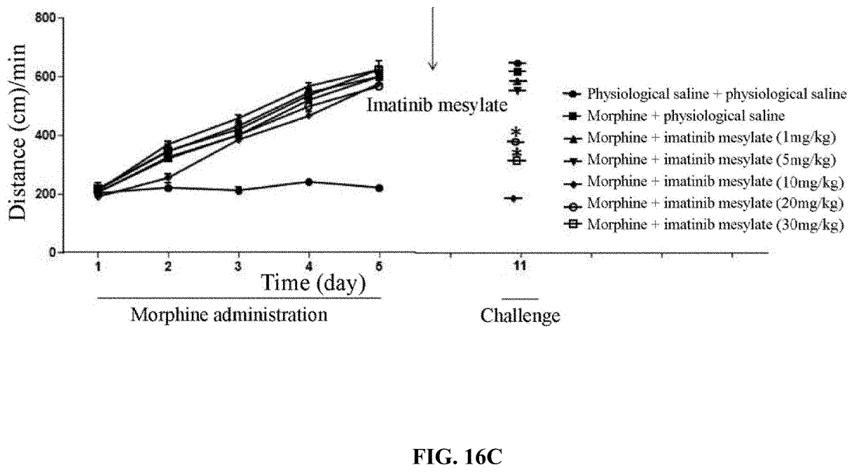

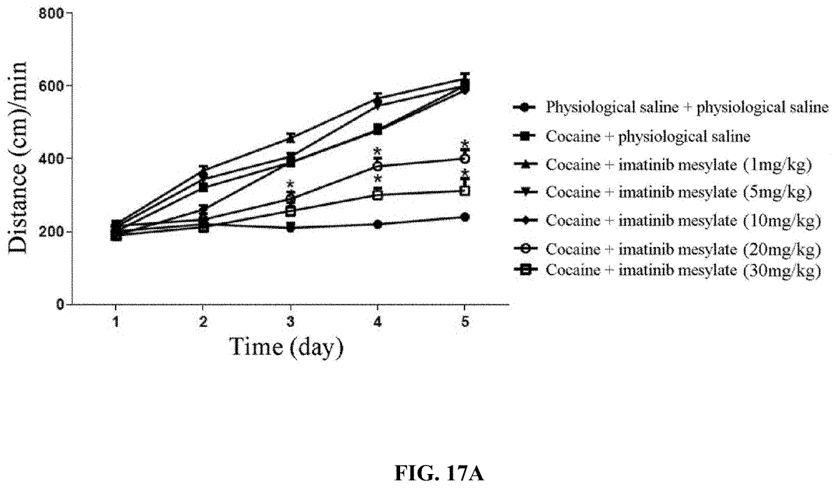

The results are shown in FIGS. 16A-16C. Effect on development: After 5 consecutive days of administration of morphine in rats, the rats given imatinib mesylate at a dose of 30 mg/kg have significant difference from the rats given physiological saline intervention, where the development of morphine sensitization in rats is inhibited.

Effects on expression: After subcutaneous injection of morphine on day 11, the rats given imatinib mesylate at a dose of 30 mg/kg are observed to have significant difference from the rats given physiological saline intervention, where the expression of morphine sensitization in rats is inhibited.

### Example 14

Dose-effect of imatinib mesylate on development and expression of cocaine sensitization in rats

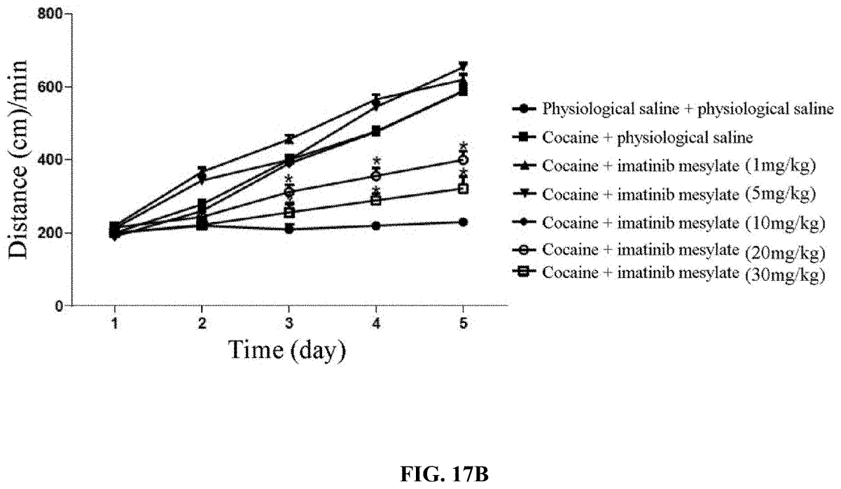

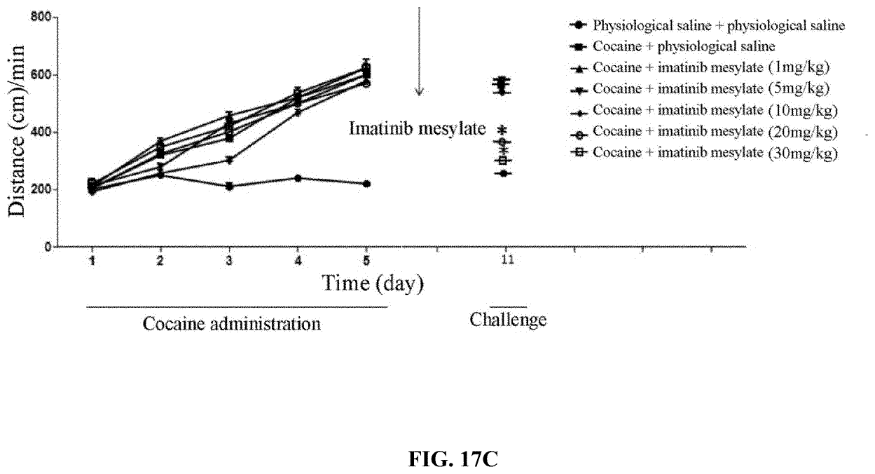

As described in Example 13, the addiction substance was cocaine in place of morphine, and similar results were obtained (FIGS. 17A-17C).

### Example 15

Effect of imatinib mesylate on development and expression of ethanol sensitization in rats

The dose of ethanol for model establishment was 0.75 g/kg, and the dose of imatinib mesylate was 1, 5, 10, 15, 20, or 30 mg/kg.

The rest process was the same as that in Example 13.

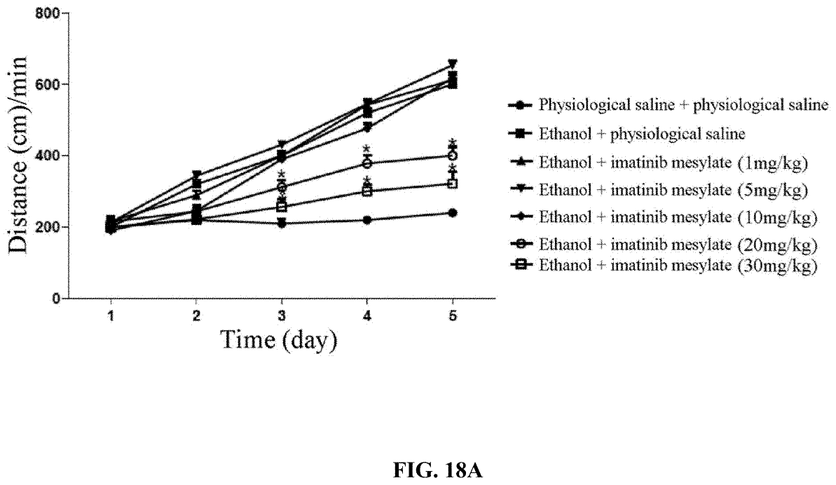

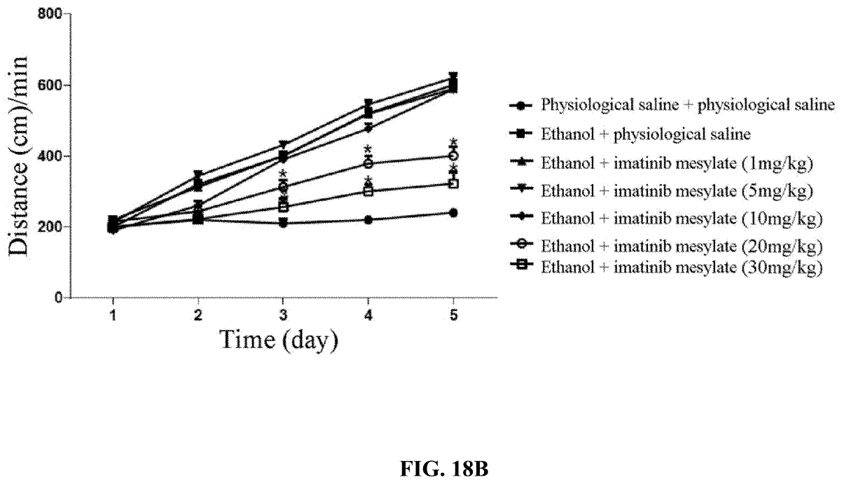

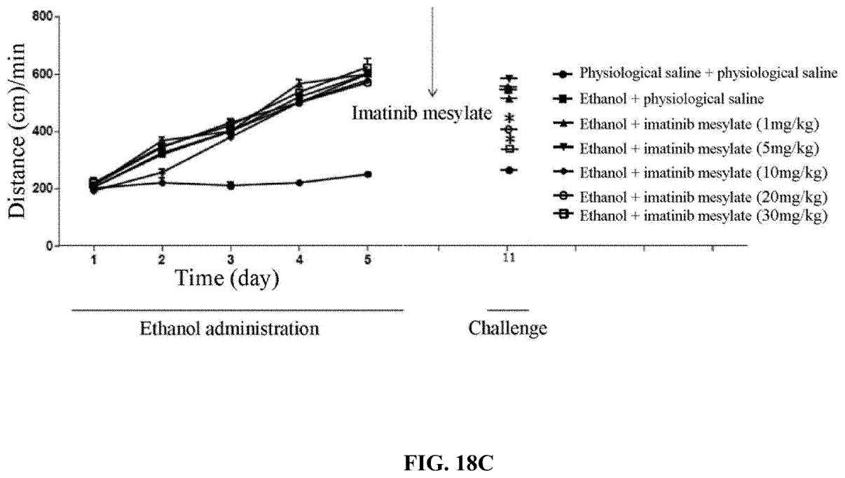

The results are shown in FIGS. 18A-18C. Effect on development: After 5 consecutive days of administration of ethanol in rats, the rats given imatinib mesylate at a dose of 15, 20, or 30 mg/kg have significant difference from the rats given physiological saline intervention, where the development of ethanol sensitization in rats is inhibited.

Effects on expression: After subcutaneous injection of ethanol on day 11, the rats given imatinib mesylate at a dose of 15, 20, or 30 mg/kg have significant difference from the rats given physiological saline intervention, where the expression of ethanol sensitization in rats is inhibited.

### Example 16

Dose-effect of imatinib mesylate on development of food addiction in rats

I. Material

Drug and reagent: self-made high-sugar and high-fat food (sucrose content: 10%, and fat content: 30%); imatinib mesylate (Novartis PharmaStein AG)

Test animals: SPF-grade male SD rats weighing 220-250 g, Provided by Hubei Laboratory Animal Research Center, Animal Certificate No.: 42010200001670, Production License No.: SCXK (Hubei) 2017-0012. Rat feed: purchased from the Laboratory Animal Center of Wuhan University.

Test instrument: the same as that in Example 1.

II. Experimental Method:

Establishment of high-sugar and high-fat food-induced CPP model

Baseline test: On day 1, the passages between three compartments were opened and the CPP program on the computer was started. The rats were transferred to the middle compartment, and allowed to move freely in the three compartments for 15 min. The time spent in each compartment was simultaneously recorded.

Groups of test animals: According to the baseline tested, the rats were divided into 12 groups (n=8):

| | Administration in the form of |
|---|---|
| Administration in combination | a compound preparation |
| High-sugar and high-fat food + | High-sugar and high-fat food + |
| physiological saline | physiological saline |
| High-sugar and high-fat food + | High-sugar and high-fat food + |
| imatinib mesylate (1, 5, 10, 20, or | imatinib mesylate (1, 5, 10, 20, or |
| 30 mg/kg) | 30 mg/kg) |

CPP training: On days 2 to 9, the passages between the three compartment were closed. On days 2, 4, 6, and 8, rats in the treatment groups were injected with different doses of imatinib mesylate (1, 5, 10, 20, or 30 mg/kg, i.p.), and then allowed to free access to food after 30 min, and transferred to the drug-paired side for 45 min. Rats in the control group were injected with normal saline (1 mL/kg, i.p.) at corresponding time point, and then allowed to free access to food and transferred to the nondrug-paired side for 45 min. On days 3, 5, 7, and 9, the rats in the treatment groups and control group were all injected with physiological saline, and allowed to access to water. The rats in the treatment group were transferred to the nondrug-paired side, the rats in the control group were transferred to the drug-paired side, and the time is 45 min in each case. The drug-paired side for each rat was constant. The rats in each group were returned to the rearing cage after the test.

CPP test: The method was the same as that in Example 1.

Test index: After the training of rats, a CPP test apparatus was used to test the addition to high-sugar and high-fat food addiction, where the CPP score reflects the development of an addictive behavior in rats, and an elevated CPP score indicates the development of the addictive behavior.

III. Experimental Results

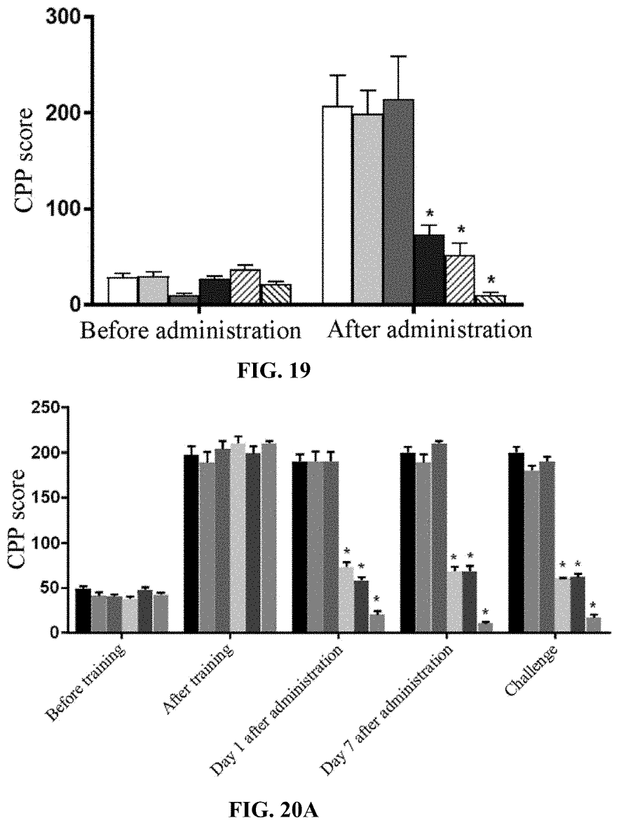

The results are shown in FIG. 19. Imatinib mesylate has a dose-dependent effect on the development of addiction to high-sugar and high-fat food. 1, 5, and 10 mg/kg of imatinib mesylate have no significant effect on the development of addiction to high-sugar and high-fat food; and 20, and 30 mg/kg of imatinib mesylate can significantly inhibit the development of addiction to high-sugar and high-fat food in rats.

### Example 17

Dose-effect of imatinib mesylate on reconsolidation after development of addiction to high-sugar and high-fat food and relapse after withdrawal

I. Material

Drug and reagent: self-made high-sugar and high-fat food (sucrose content: 10%, and fat content: 30%); imatinib mesylate (Novartis PharmaStein AG)

Test animals: SPF-grade male SD rats weighing 220-250 g, Provided by Hubei Laboratory Animal Research Center, Animal Certificate No.: 42010200001673, Production License No.: SCXK (Hubei) 2017-0012. Rat feed: purchased from the Laboratory Animal Center of Wuhan University.

Test instrument: the same as that in Example 1.

II. Experimental Method:

(1) Establishment of High-Sugar and High-Fat Food-Induced CPP Model

Baseline test: On day 1, the passages between three compartments were opened and the CPP program on the computer was started. The rats were transferred to the middle compartment, and allowed to move freely in the three compartments for 15 min. The time spent in each compartment was simultaneously recorded.

According to the baseline test result, the rats were divided into 18 groups (n=8):

| Induction with substance | Induction with | |
|---|---|---|
| clue (administration in | environmental | Direct |
| combination) | clue | administration |
| High-sugar and high-fat | Physiological | Physiological |
| food + physiological saline | saline | saline |
| High-sugar and high-fat | Imatinib mesylate | Imatinib mesylate |
| food + imatinib mesylate | (1, 5, 10, 20, | (1, 5, 10, 20, |
| (1, 5, 10, 20, and 30 mg/kg) | and 30 mg/kg) | and 30 mg/kg) |

CPP training: On days 2 to 9, the passages between the three compartment were closed. On days 2, 4, 6, and 8, the rats in the treatment groups were allowed to free access to high-sugar and high-fat food, and transferred to the drug-paired side for 45 min; and the rats in the control group were allowed to free access to water and transferred to the nondrug-paired side for 45 min. On days 3, 5, 7, and 9, the rats in the treatment groups and control group were all allowed to free access to water. The rats in the treatment group were transferred to the nondrug-paired side, the rats in the control group were transferred to the drug-paired side, and the time was 45 min in each case. The drug-paired side for each rat was constant. The rats in each group were returned to the rearing cage after the test.

CPP test: The CPP test was performed on day 10. Similar to the baseline test, the passages between three compartments were opened, the rats did not receive any treatment, and the CPP program on the computer was started. The rats were transferred to the middle compartment, and allowed to move freely in the three compartments for 15 min. The time spent in each compartment was simultaneously recorded. The CPP score is defined as difference between the time spent in the drug-paired compartment and the time spent in the nondrug-paired compartment. The CPP score of rats before and after training in the CPP test apparatus were compared to determine whether CPP was developed in the rats, and the rats where CPP was not developed were excluded according to the CPP score tested after training. Then the rats were grouped.

(2) Establishment of Drug-Seeking Behavior Model Induced by Environmental Clue or Unconditioned Stimulus

On day 11 of the test, for induction with substance clue, a small amount of high-sugar and high-fat food was given and then different doses of imatinib mesylate (1, 5, 10, 20, and 30 mg/kg) were injected; for induction with environmental clue, the rats were exposed in the drug-paired compartment for 15 min and then imatinib mesylate (1, 5, 10, 20, and 30 mg/kg) were injected; and for the rats receiving direct administration, they were directly given different doses of imatinib mesylate (1, 5, 10, 20, and 30 mg/kg) without induction. The rats were then all returned to the rearing cage.

(3) Retest of CPP

On days 1 and 7 after the administration of imatinib mesylate, that is, days 12 and 18 of the test, the rats' preference for the drug-paired compartment within 15 min was tested, which was similar to the baseline test. On the intermediate days 12 and 17, the rats did not receive any treatment. On day 19, the rats were primed by giving a small amount of high-sugar and high-fat food, and tested for the CPP score.

Test index: After the training of rats, a CPP test apparatus was used to test the addition to high-sugar and high-fat food addiction, where the CPP score reflects the development of an addictive behavior in rats, and an elevated CPP score indicates the development of the addictive behavior.

III. Experimental Results

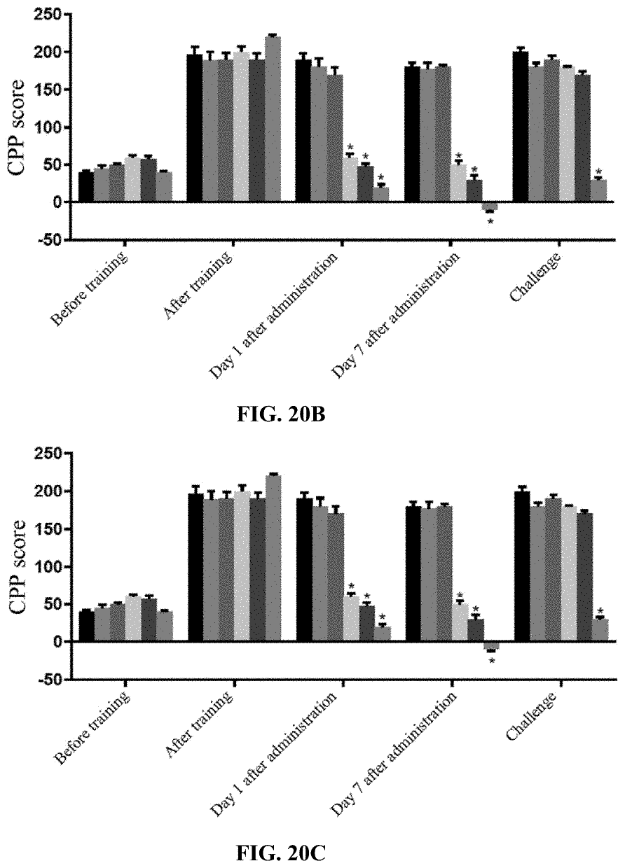

The results are shown in FIGS. 20A-20C. For the rats induced with substance cues, environmental cue, or receiving direct administration without induction, CPP still exists in rats without treatment; and treatment with 10, 20, and 30 mg/kg of imatinib mesylate can inhibit the food-seeking behavior. However, after 1 week, for the rats induced with environmental clue, only 30 mg/kg of imatinib mesylate can prevent the rats from being primed; and for the rats induced with substance clue, 10, 20, and 30 mg/kg of imatinib mesylate can prevent the rats from being primed; and for the rats receiving direct administration, the use of all doses of imatinib mesylate cannot prevent the rats from being primed. The results indicate that the administration of imatinib mesylate after induction with substance cue is more effective than the administration after induction with environmental clue or the direct administration.

### Example 18

Dose-effect of imatinib mesylate on gambling behavior in rats under gambling task conditions

I. Material

Test animals: SPF-grade male SD rats weighing 275-300 g, Provided by Hubei Laboratory Animal Research Center, Animal Certificate No.: 42010200001574, Production License No.: SCXK (Hubei) 2017-0012. Rat feed: purchased from the Laboratory Animal Center of Wuhan University.

Test instrument: Five-hole operating chambers. Each operant chamber was enclosed in a ventilated sound-attenuating cabinet. Each operant chamber was fitted with an array of five response holes positioned 2 cm above the floor A stimulus light was set at the back of each hole. Nose-poke responses into these apertures were detected by a horizontal infrared beam. A food magazine, also equipped with an infrared beam and a tray light, was located in the middle of the opposite wall and sucrose pellets of 45 mg could be delivered into it from an external pellet dispenser. Chambers could be illuminated using a house light, and were controlled by software written in Med PC by CAW running on an IBM-compatible computer.

II. Experimental Method:

Groups of test animals: 6 groups in total (n=10), including physiological saline group, and groups with imatinib mesylate of 1, 5, 10, 20, and 30 mg/kg.

Establishment of gambling behavior model in rats: Animals were first habituated to the operant chambers over two daily 30-min sessions, during which sucrose pellets were transferred to the response holes and in the food magazine. After habituation, animals were then trained to make a nose-poke response into an illuminated response hole within 10 s to earn reward where the spatial location of the stimulus light varied between trials across holes 1, 2, 4, and 5. Each session consisted of 100 trials and lasted approximately 30 min. Animals were then trained 7 times on a forced-choice version of the rGT (or variant thereof in the case of the control groups) before moving on to the full free choice task. This ensured all animals had equal experience with all of the four reinforcement contingencies, and aimed to prevent simple biases toward a particular hole from developing. The percentage of trials on which an animal chose a particular option was calculated according to the following formula: number of choices of a particular option/number of total choices×100 in the reference (Di C P, Manvich D F, Pushparaj A, et al. Effects of disulfiram on choice behavior in a rodent gambling task: association with catecholamine levels[J]. Psychopharmacology, 2018, 235(1): 23-35). Each session lasted for 30 min. Subjects made a nose-poke response in the illuminated food magazine. This response extinguished the tray light and triggered the start of a 5-s inter-trial interval (ITT). At the end of the ITI, holes 1, 2, 4, and 5 were illuminated for 10 s (in the forced-choice version of the task used in training, only one hole was illuminated). if animals failed to respond within 10 s, the trial was scored as an omission, at which point the tray-light was re-illuminated and animals could start a new trial.

| Option 1 | Option 2 | Option 3 | Option 4 |
|---|---|---|---|
| 1 sucrose pellet, | 2 sucrose pellets, | 3 sucrose pellets, | 4 sucrose pellets, |
| p = 0.9 | p = 0.8 | p = 0.5 | p = 0.4 |
| 5s punishment | 10s punishment | 30s punishment | 40s punishment |
| time, | time, | time, | time, |
| p = 0.1 | p = 0.2 | p = 0.5 | p = 0.6 |

Interpretation of the above table: Four holes in the test chamber were respectively set with different reward probability and punishment probability, as well as corresponding sucrose pellets for reward and punishment time. There was no food reward during the punishment time, which indicates that in 30 minutes, the best gain will get if only option 2 is chosen after completing a series of choices.

The training was continued until the baseline of the rats was stable, and the rats in the rGT group always showed a preference for the choice of two sucrose pellets, that is, the best gain of choices. The overall tendency was P2>P4>P1>P3, but in fact, the best ranking of choices was P2>P1>P3>P4.

After training, the rats were administered. On day 1 after the baseline was stabilized, the rats for induction with environmental clue were transferred to the chamber as did in the habituation period, but the test was not started. Then imatinib mesylate (1, 5, 10, 20, or 30 mg/kg, i.p.) or physiological saline (1 mL/kg, i.p.) was administered. The rats in the group receiving direct administration were directly administered with imatinib mesylate (1, 5, 10, 20, or 30 mg/kg, i.p.) or physiological saline (1 mL/kg, i.p.) without being transferred to the chamber. Then all rats were returned to their cages. On day 1 after administration, a behavioral test was conducted. On day 7 after administration, a behavioral test was conducted again.

III. Experimental Results

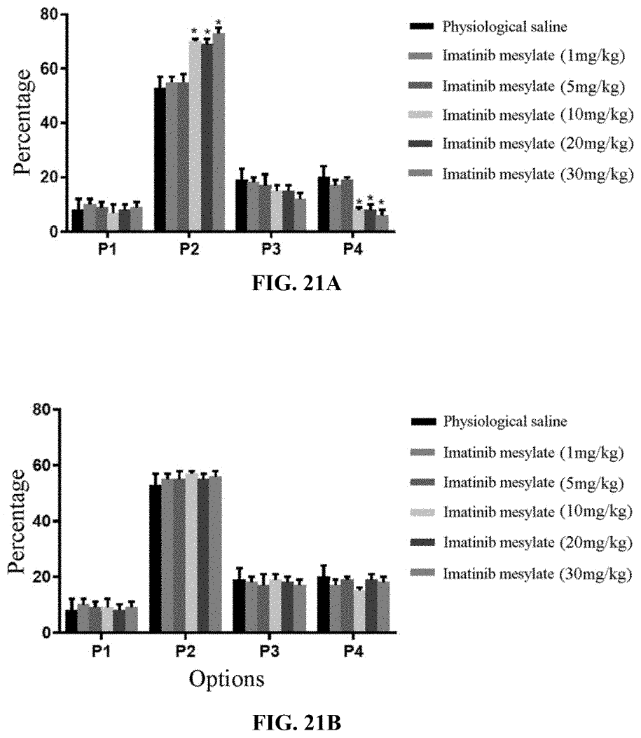

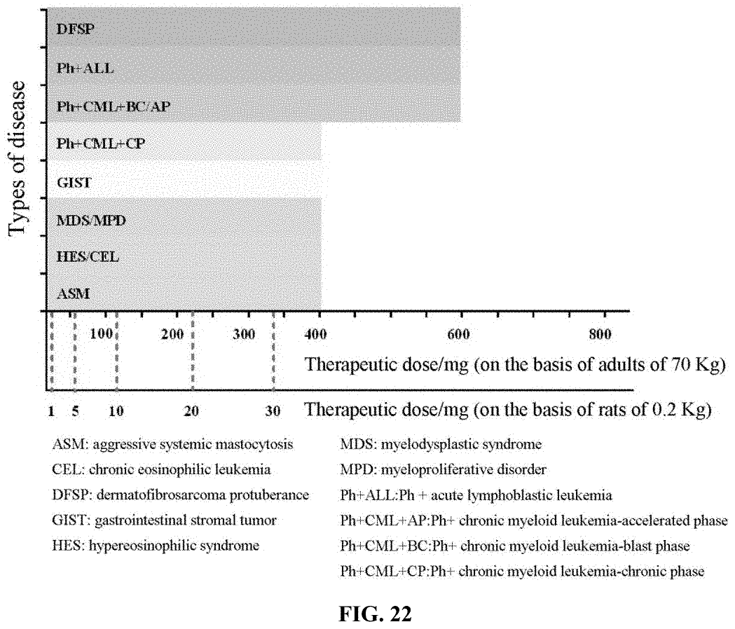

The results are shown in FIGS. 21A-21B. After exposure to environmental clue, administration of imatinib mesylate at a dose of 10, 20, and 30 mg/kg significantly increase the choice of the best option, that is, P2, and reduce the choice of option P, compared to the control group. However, no significant effect of imatinib mesylate is found at any dose in rats receiving direct administration. The results show that for the gambling task of rats, after the baseline is stabilized, induction of the animals with an environmental cue before administration enhances the improvement compared to direct administration.

Conclusion: The results of the above test show that the addictive substance specifically activates the c-kit receptor and its downstream ERK, AKT, and PKMzeta signaling molecules in neurons in nucleus accumbens instead of other brain regions; and imatinib mesylate inhibits the psychological craving behavior by blocking the c-kit receptor and its signaling pathway, thereby preventing the addiction to addictive substances and addictive behaviors and controlling the relapse. Similarly, imatinib or a derivative thereof administered in combination with or in a compound preparation with an addictive substance or an addictive behavior-related clue can prevent and treat the addiction and relapse after withdrawal.

Examples 19 to 23 provide use of imatinib or a derivative thereof with nicotine or an analogue thereof in the addiction treatment. Materials and reagents involved in the examples are commercially available, unless otherwise specified.

### Example 19

Effect of imatinib mesylate with nicotine at a certain dose ratio on development of ethanol addiction in rats and molecular mechanism

Test 19: Effect of imatinib mesylate with nicotine at a certain dose ratio on development of ethanol addiction in rats

I. Material

Drug: nicotine (Apexbio), imatinib mesylate (Selleck Chemicals).

Test animals: SPF-grade male SD rats weighing 180-220 g, provided by the Laboratory Animal Center of China Three Gorges University. Animal Certificate No.: 42010200001750, Production License No.: SCXK (Hubei) 2017-0012. Rat feed: purchased from the Laboratory Animal Center of Wuhan University.

Test instrument: Conditional place preference (CPP) test apparatus (developed by the Institute of Chinese Materia Medica, China Academy of Chinese Medical Sciences). The test was automatically controlled by a computer. The apparatus is a three-compartmented CPP box, inducing two side compartments and a middle compartment, where the three compartments are separated by removable partitions and are black internally and externally. The compartments A and B are located at two sides of the middle box and have the same size. 9 yellow light-emitting diodes are provided on a side wall of the compartment A to form a square array, and the bottom plate is formed of stainless steel strips. The bottom plate of the compartment B is formed of a stainless steel mesh. The data regarding time of rats spent in each compartment and the number of entries can be transmitted to the computer, and the behavioral data is automatically collected and recorded.

II. Experimental Method:

Baseline test: On day 1, the partitions were removed to open the passages between the three compartments, and the CPP program on the computer was started. The rats were transferred to the middle compartment, and allowed to move freely in the three compartments for 15 min. The time spent in each compartment was simultaneously recorded. According to the test results, some rats were excluded, and the remaining rats were divided into 6 groups (nicotine+physiological saline group, nicotine+imatinib mesylate group (1 mg/kg), nicotine+imatinib mesylate group (5 mg/kg), nicotine+imatinib mesylate group (10 mg/kg), nicotine+imatinib mesylate group (20 mg/kg), nicotine+imatinib mesylate group (30 mg/kg)) each group having ten animals, assigned to a drug-paired side or a nondrug-paired side.

CPP training: On days 2 to 9, the passages between the three compartment were closed. Imatinib mesylate was administered in combination with or in the form of a compound preparation with nicotine. On days 2, 4, 6, and 8, the rats in each group were intraperitoneally injected with different doses of imatinib mesylate (1, 5, 10, 20, and 30 mg/kg, i.p.) or physiological saline (1 mL/kg, i.p.), and then subcutaneously injected with nicotine (0.25, and 0.5 mg/kg, s.c.) 30 min later; or the rats in each group were subcutaneously injected with a mixed agent of imatinib mesylate (1, 5, 10, 20, and 30 mg/kg, s.c.) or physiological saline (1 mL/kg, s.c.) with nicotine (0.25, and 0.5 mg/kg, s.c.) at various dose ratios. Then the rats were transferred to the drug-paired side for 45 min. On days 3, 5, 7, and 9, the rats in nicotine group were intraperitoneally injected with physiological saline (1 mL/kg, i.p.), and then subcutaneously injected with physiological saline (1 mL/kg, s.c.) 30 min later; or the rats in each group were subcutaneously injected with physiological saline (1 mL/kg, s.c.), and transferred to the nondrug-paired side for 45 min. The drug-paired side for each rat was constant. The rats in each group were returned to the rearing cage after the test.

Test of nicotine-induced CPP: On day 10, CPP was tested. Similar to the baseline test, the passages between the three compartments were opened and the rats did not receive any injection. the CPP program on the computer was started. The rats were transferred to the middle compartment, and allowed to move freely in the compartments for 15 min. The time spent in each compartment was simultaneously recorded. CPP score is the difference between the time spent in the drug-paired side and the time spent in the nondrug-paired side. The CPP scores tested before and after training in the CPP test apparatus were compared to determine whether CPP is developed in the rats.

III. Experimental Results

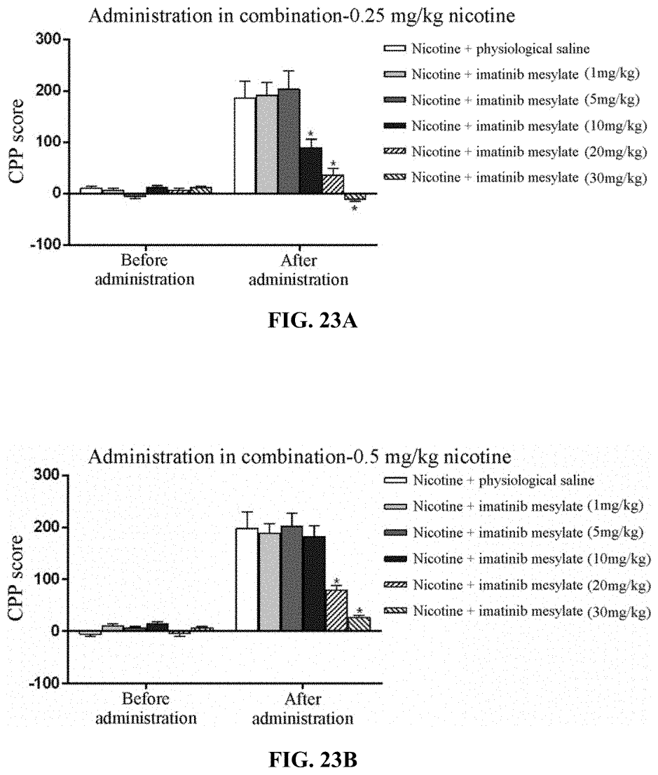

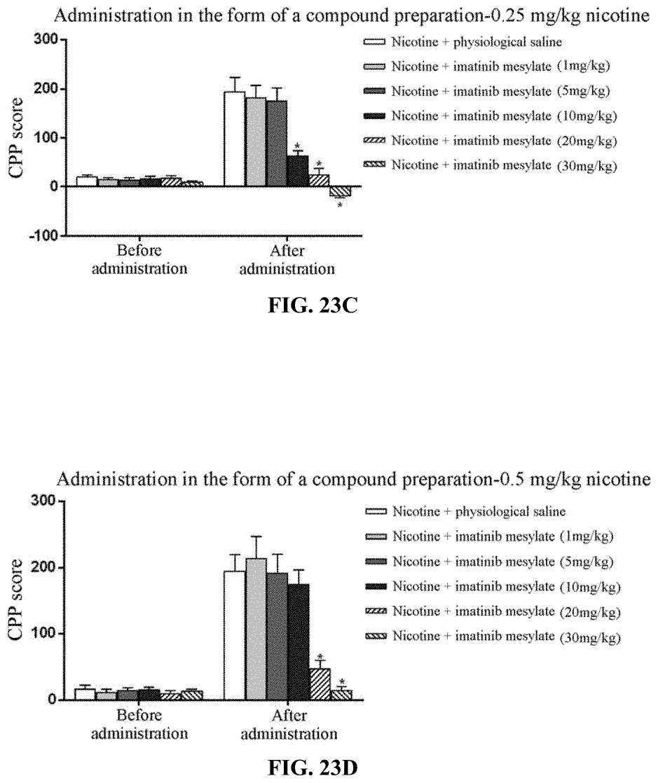

The results are shown in FIG. 23A. The CPP score in the groups receiving combined administration of imatinib mesylate+nicotine has significant difference from that in the physiological saline+nicotine group, and is dose-ratio dependent. Imatinib sulfonate intraperitoneally injected at a dose of 10, 20, and 30 mg/kg can inhibit the development of CPP in rats induced by 0.25 mg/kg of nicotine; and 1, and 5 mg/kg of imatinib mesylate do not have this inhibitory effect. CPP still exists in rats not receiving treatment with imatinib mesylate. FIG. 23B shows that imatinib sulfonate intraperitoneally injected at a dose of 20, and 30 mg/kg can inhibit the development of CPP in rats induced by 0.5 mg/kg of nicotine; and 1, 5, and 10 mg/kg of imatinib mesylate do not have this inhibitory effect. The results indicate that imatinib mesylate can inhibit the development of nicotine addiction in rats when the dose ratio of imatinib mesylate to nicotine is 40:1 or greater. Similarly, similar results are obtained for the administration of imatinib mesylate and nicotine in the form of compound preparation in FIGS. 23C and 23D.

Test 20: New molecular mechanism of imatinib mesylate in preventing nicotine addiction

It can be known from the results of test 19 that regardless of administration in combination or in the form of a compound preparation, imatinib mesylate (10-30 mg/kg) has different inhibitions on the development of nicotine-induced CPP. In this test, immunohistochemistry and multicolor immunofluorescence co-labeling were used to detect the changes of phosphorylation level of c-kit and its downstream activated target molecules in the brain region of drug reward, to determine the new molecular mechanism of nicotine addiction and the mechanism of action of imatinib mesylate in controlling addiction.

I. Material

Drug: nicotine (Apexbio), imatinib mesylate (Selleck Chemicals).

Test animals: SPF-grade male SD rats weighing 180-220 g, provided by the Laboratory Animal Center of China Three Gorges University. Animal Certificate No.: 42010200001704, Production License No.: SCXK (Hubei) 2017-0012. Rat feed: purchased from the Laboratory Animal Center of Wuhan University.

II. Experimental Method:

The animals were randomized to a physiological saline+physiological saline group, a physiological saline+imatinib mesylate group, a nicotine+physiological saline group, and a nicotine+imatinib mesylate group, each group having ten animals. The rats in each group were intraperitoneally administered with physiological saline (1 mL/kg, i.p.) or imatinib mesylate (30 mg/kg, i.p.), and then subcutaneously injected with nicotine (0.5 mg/kg, s.c.) 30 min later. After 60 min, the changes of c-kit activity in the mesolimbic dopamine system, including VTA, nucleus accumbens, amygdala, hippocampus, and prefrontal cortex were observed, the changes in phosphorylation level of c-kit were observed by immunohistochemistry and the downstream activated target molecules were determined by multicolor immunofluorescence co-labeling, to determine the new molecular mechanism of nicotine addiction and the mechanism of action of imatinib mesylate in controlling addiction.

III. Experimental Results

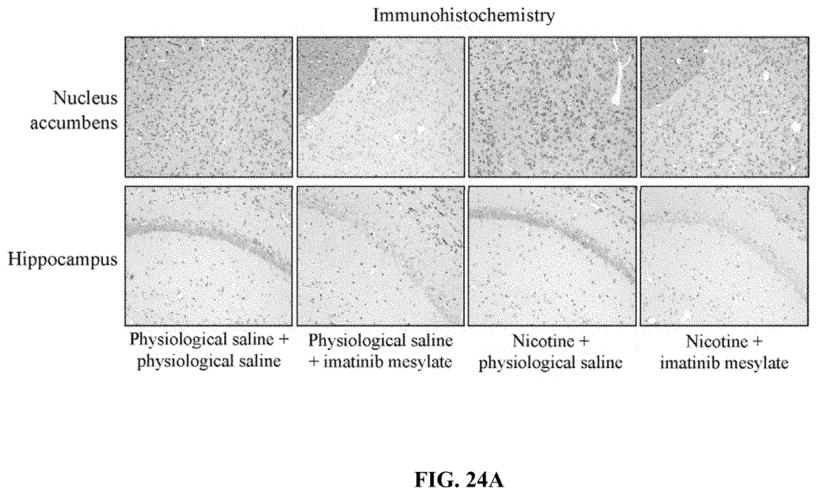

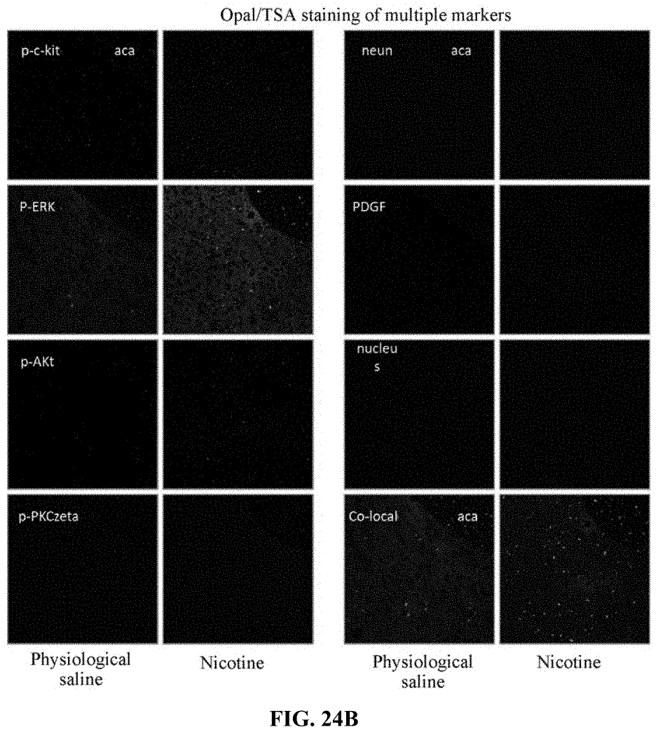

The results are shown in FIGS. 24A-24B, After acute nicotine administration, the results of immunohistochemistry and multi-color immunofluorescence co-labeling show that the c-kit receptor, and multiple downstream signaling molecules such as PKC, PI3K-AKT, ERK, and other regulatory kinases, protein expressions, gene expressions, and regulation and initiation of the process of nicotine reward, memory and neuroplasticity in neurons in nucleus accumbens instead of in other brain regions are specifically activated. The results suggest that the c-kit receptor in nucleus accumbens is a brain region specifically activated by acute nicotine ministration; and imatinib mesylate inhibits multiple signaling molecules such as PKC, PI3K-AKT, ERK, and other regulatory kinases, protein expressions, gene expressions, and regulation and initiation of the process of nicotine reward, memory and neuroplasticity by blocking the c-kit receptor, to achieve the effect of preventing nicotine addiction.

### Example 20

Effect of imatinib mesylate on drug-seeking behavior in rats after nicotine addiction after unconditioned stimulus-induced re-arousal of memory

According to Examples 19, imatinib or its derivative imatinib mesylate inhibits the development of nicotine addiction in rats by inhibiting the phosphorylation activity of c-kit in nucleus accumbens. In this example, a CPP model was established to investigate the effect of imatinib mesylate with nicotine at a certain dose ratio on the relapse after nicotine addiction in rats.

I. Material

Drug: nicotine (Apexbio), imatinib mesylate (Selleck Chemicals).

Test animals: SPF-grade male SD rats weighing 180-220 g, provided by the Laboratory Animal Center of China Three Gorges University. Animal Certificate No.: 42010200001855, Production License No.: SCXK (Hubei) 2017-0012. Rat feed: purchased from the Laboratory Animal Center of Wuhan University.

Test instrument: the same as that in Example 19.

II. Experimental Method:

Baseline test: The method was the same as that in Example 19.

CPP training: On days 2 to 9, the passages between the three compartment were closed. The rats were subcutaneously injected with 0.25, or 0.5 mg/kg of nicotine. Then the rats were transferred to the drug-paired side for 45 min. On days 3, 5, 7, and 9, each rat was subcutaneously injected with physiological saline (1 mL/kg, s.c.) and transferred to the nondrug-paired side for 45 min. The drug-paired side for each rat was constant. The rats in each group were returned to the rearing cage after the test.

Test of nicotine-induced CPP: The method was the same as that in Example 19.

Model establishment: On day 11, the rats were divided into 12 groups according to the CPP score, including:

| | |
|---|---|
| Induction with unconditioned | Induction with unconditioned |
| stimulus (where the dose of nicotine | stimulus (where the dose of nicotine |
| for training CPP is 0.5 mg/kg) | for training CPP is 0.5 mg/kg) |
| Physiological saline | Physiological saline |
| Imatinib mesylate (1, 5, 10, 20, | Imatinib mesylate (1, 5, 10, 20, |
| and 30 mg/kg) | and 30 mg/kg) |

The rats were exposed in the drug-paired side or subcutaneously injected with 0.25 or 0.5 mg/kg of nicotine and then intraperitoneally injected with different doses of imatinib mesylate (1, 5, 10, 20, and 30 mg/kg, i.p.) or physiological saline (1 mL/kg, i.p.) 15 min later; or each rat was subcutaneously injected with a mixed agent of imatinib mesylate (1, 5, 10, 20, and 30 mg/kg, s.c.) or physiological saline (1 mL/kg, s.c.) with nicotine (0.25, and 0.5 mg/kg, s.c.) at various dose ratios.

On day 11 of the test, the rats were injected with 0.15 mg/kg of nicotine, then transferred to the drug-paired side for 15 min, and then administered with various doses of imatinib mesylate (1, 5, 10, 20, and 30 mg/kg, i.p.) or physiological saline (1 mL/kg, i.p.); or the rats were injected with a mixed agent of nicotine with imatinib mesylate and then transferred to the drug-paired side for 15 min.

Retest of nicotine-induced CPP: On days 1 and 7 after the administration of imatinib mesylate, that is, days 12 and 18 of the test, the rats' preference for the drug-paired compartment was tested, which was similar to the baseline test. On the intermediate days 13 and 17, the rats did not receive any treatment.

III. Experimental Results

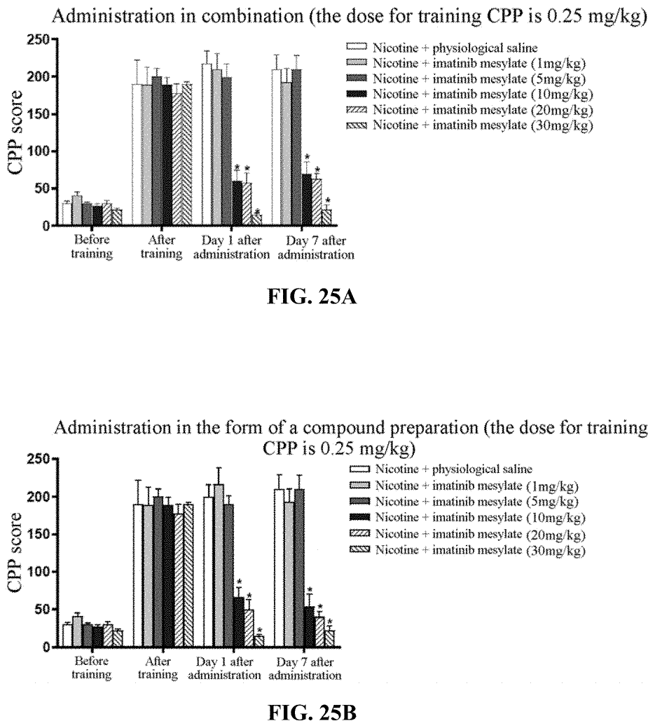

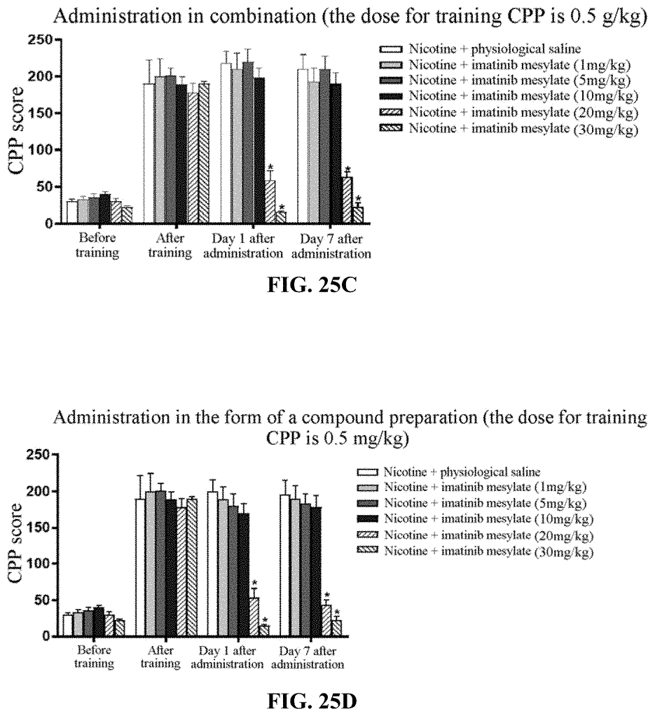

The results are shown in FIGS. 25A and 25B. Regardless of administration in combination or in the form of a compound preparation, for rats receiving training of CPP with 0.25 mg/kg of nicotine, 10, 20, and 30 mg/kg of imatinib mesylate have different inhibitions on the nicotine-seeking behavior caused by induction with conditioned clue. FIGS. 25C and 25D show that for rats receiving training of CPP with 0.5 mg/kg of nicotine, regardless of administration in combination or in the form of a compound preparation, 20 and 30 mg/kg of imatinib mesylate have obvious inhibition on the drug-seeking behavior. The results indicate that the drug-seeking behavior caused by unconditional cue after nicotine addiction can be effectively treated, where the ratio of dose for training nicotine-induced CPP to the dose of imatinib mesylate needs to be 1:40 or less, and this effect is present regardless of administration in combination or in the form of a compound preparation.

### Example 21

Effect of imatinib mesylate administered after induction with unconditioned stimulus (in combination, or in the form of a compound preparation), after induction with environmental clue, or directly on the drug-seeking behavior after ethanol addiction and relapse after withdrawal in rats.

I. Material

Drug: nicotine (Apexbio), imatinib mesylate (Selleck Chemicals).

Test animals: SPF-grade male SD rats weighing 180-220 g, provided by the Laboratory Animal Center of China Three Gorges University. Animal Certificate No.: 42010200001721, Production License No.: SCXK (Hubei) 2017-0012. Rat feed: purchased from the Laboratory Animal Center of Wuhan University.

Test instrument: The method was the same as that in Example 19.

Groups:

| | | | |
|---|---|---|---|
| Unconditioned | Unconditioned | Induction with | Direct |
| stimulus | stimulus | environmental | administration |
| (administration | (administration | clue | |
| in combination) | in the form of | | |
| | a compound | | |
| | preparation) | | |
| Nicotine (0.1, | Nicotine (0.1, | Physiological | Physiological |
| and 0.15 mg/kg | and 0.15 mg/kg | saline | saline |
| for induction) + | for induction) + | | |
| physiological | physiological | | |
| saline | saline | | |
| Nicotine (0.1, | Nicotine (0.1, | Imatinib | Imatinib |
| and 0.15 mg/kg | and 0.15 mg/kg | mesylate | mesylate |
| for induction) + | for induction) + | (20, and 30 | (20, and 30 |
| imatinib | imatinib | mg/kg) | mg/kg) |
| mesylate (20, | mesylate (20, | | |
| and 30 mg/kg) | and 30 mg/kg) | | |

CPP training: On days 2 to 9, the passages between the three compartment were closed. On days 2, 4, and 6, and 8, rats in the treatment groups were subcutaneously injected with nicotine (0.5 mg/kg), and then transferred to the drug-paired side for 45 min; and rats in the control group were subcutaneously injected with physiological saline (1 mL/kg) and then transferred to the nondrug-paired side for 45 min. On days 3, 5, 7, and 9, the rats in the treatment groups and control group were all injected with physiological saline (1 mL/kg). The rats in the treatment group were transferred to the nondrug-paired side, the rats in the control group were transferred to the drug-paired side, and the time is 45 min in each case. The drug-paired side for each rat was constant. The rats in each group were returned to the rearing cage after the test.

Test of morphine-induced CPP: The method was the same as that in Example 19.

(2) Test of Effect of Imatinib Mesylate on Drug-Seeking Behavior

On day 11 of the test, for induction with substance clue, the rats in the group receiving administration in combination were injected with nicotine (0.1, or 0.15 mg/kg, s.c.) or physiological saline (1 mL/kg, s.c.) and then intraperitoneally administered with 20, or 30 mg/kg of imatinib mesylate 30 min later; and the rats in the group receiving administration in the form of a compound preparation were administered with a mixture of 30 mg/kg of imatinib mesylate with nicotine (0.1, or 0.15 mg/kg, i.p.) or physiological saline (1 mL/kg). The rats for induction with environmental clue were returned to the drug-paired side for 15 min, and then intraperitoneally injected with imatinib mesylate at a dose of 20 or 30 mg/kg, or physiological saline at a dose of 1 mL/kg. The rats without induction were directly administered with imatinib mesylate (20, or 30 mg/kg, i.p.) or physiological saline (1 mL/kg, i.p.). The rats were then returned to the rearing cage, and tested for CPP after 24 hrs.

(3) Retest of Morphine-Induced CPP

On days 1 and 7 after the administration of imatinib mesylate, that is, days 12 and 18 of the test, the rats' preference for the drug-paired compartment within 15 min was tested, which was similar to the baseline test. On day 19, the rats were primed with a small dose of nicotine (0.15 mg/kg, s.c.). After nicotine injection, the rats were transferred to the middle compartment, and subjected to CPP tests for 15 min. The rats did not receive any treatment during the non-test period.

III. Experimental Results

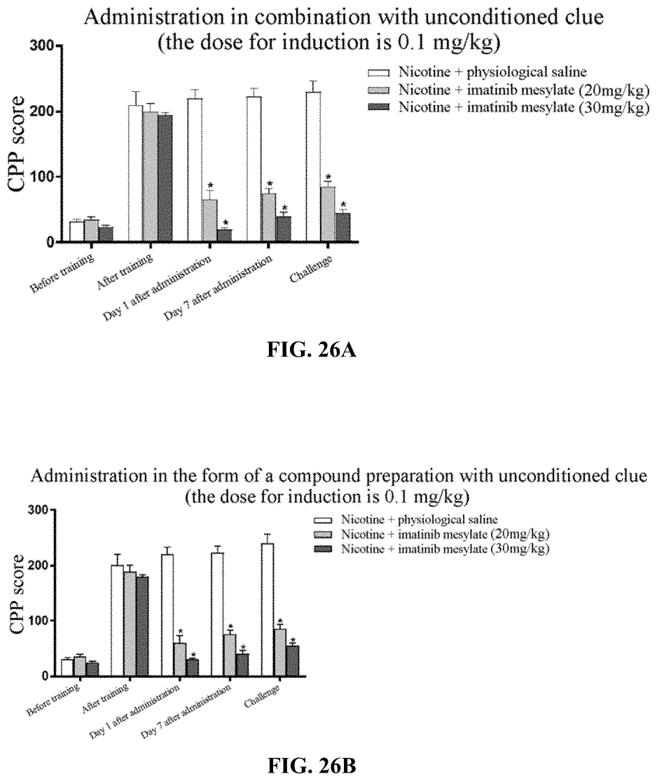

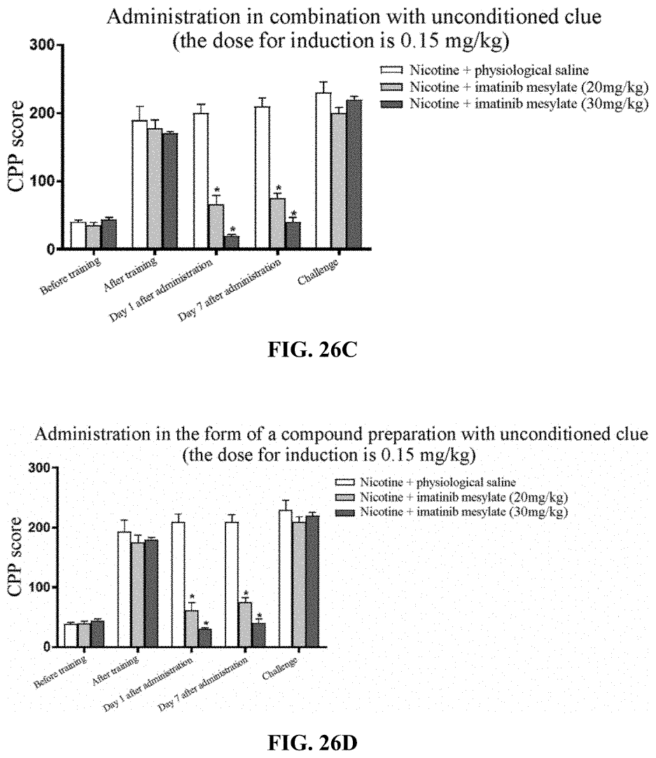

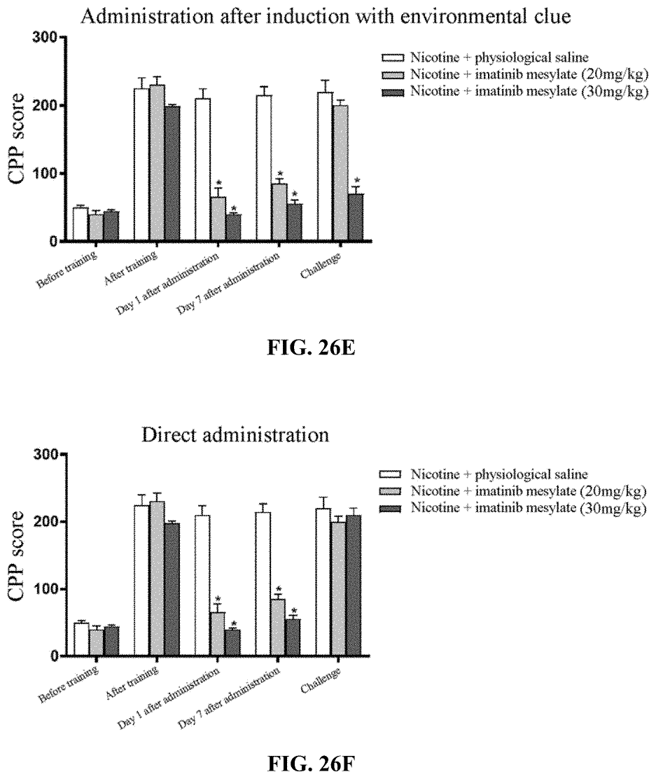

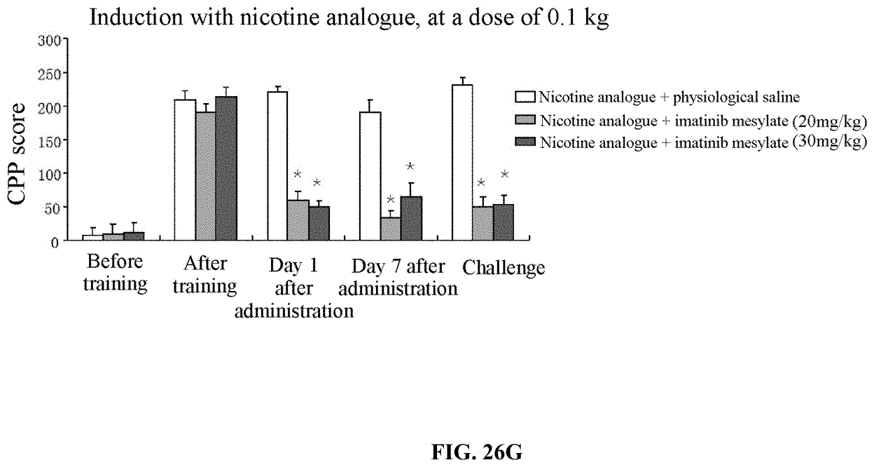

The results are shown in FIGS. 26A-26G. The treatment groups have significant difference from the control groups. 20 and 30 mg/kg of imatinib mesylate administered can significantly reduce the CPP in all treatment groups. CPP still exists in rats administered at other doses. The results indicate that imatinib mesylate administered with nicotine at a ratio of 40:1 can block the drug-seeking behavior after addiction, and suppress the uncontrollable craving. After challenge with a small dose of nicotine or an analogue thereof, for induction with unconditioned stimulus both in the group receiving administration in combination and in the group receiving administration in the form of a compound preparation, when the dose of nicotine for induction is 0.1 mg/kg, the rats in groups receiving 20, and 30 mg/kg of imatinib mesylate (including administration in combination and administration in the form of a compound preparation) are not primed; for induction with environmental clue, only the rats in the group administered with 30 mg/kg of imatinib mesylate are not primed; and the rats in the group receiving direct administration of imatinib mesylate are all primed. The results indicate that relapse can be prevented when administered after induction with unconditioned stimulus (in combination and in the form of a compound preparation). All the doses can inhibit the drug-seeking behavior, prevent the rats from being primed, and well control the relapse only when the ratio of dose of imatinib mesylate used to the dose of nicotine for training is 40:1 or greater, and the ratio of dose of nicotine for unconditioned stimulus to the dose of nicotine for training is 3:10 or less. When administered after induction with environmental clue (in combination and in the form of a compound preparation), the drug-seeking behavior can only be partially prevented, and the effect of preventing relapse is weak. The direct administration of imatinib mesylate can block the drug-seeking behavior only when the dose of imatinib mesylate is as high as 30 mg/kg, and the effect of preventing relapse is extremely weak.

### Example 22

Effect of imatinib mesylate with nicotine at a certain dose ratio on nicotine withdrawal symptoms

According to Examples 19, imatinib or its derivative imatinib mesylate inhibits the development of nicotine addiction and relapse in rats by inhibiting the phosphorylation activity of c-kit in nucleus accumbens. In this example, a nicotine addiction model was established to investigate the effect of imatinib mesylate with nicotine at a certain dose ratio on nicotine withdrawal symptoms in rats.

I. Material

Drug: nicotine (Apexbio), imatinib mesylate (Selleck Chemicals).

Test animals: SPF-grade male SD rats weighing 180-220 g, provided by the Laboratory Animal Center of China Three Gorges University. Animal Certificate No.: 42010200001750, Production License No.: SCXK (Hubei) 2017-0012. Rat feed: purchased from the Laboratory Animal Center of Wuhan University.

Test instrument: Conditional place preference (CPP) test apparatus (the same as that in Example 19); and spontaneous activity detecting apparatus (developed by the Institute of Chinese Materia Medica, China Academy of Chinese Medical Sciences). The test was automatically controlled by a computer. The apparatus includes four spontaneous activity observation chamber, a video synthesizer, a video pattern sampling card, and analysis software. The system performs video tracking of the rat activity and automatically records the rat activity track and activity times. The spontaneous activity evaluation index is the total activity times in a certain period of time (such as 60 min), and an increase in the total activity times indicates an increase in spontaneous activity.

II. Experimental Method:

Establishment of nicotine addiction model: After nicotine addiction in rats trained by 0.25 and 0.5 mg/kg of nicotine in Example 19, the rats were divided into three groups (a nicotine+physiological saline group, a nicotine+mecamylamine group (0.5 mg/kg), and a nicotine+imatinib mesylate group, each group having 10 animals). In addition, a physiological saline+physiological saline group without nicotine treatment was used as the control group.

Establishment of nicotine withdrawal model: After 24 hrs, the rats in nicotine group were intraperitoneally injected with 1, 5, 10, 20, and 30 mg/kg of imatinib mesylate; and then subcutaneously injected with 0.15 mg/kg of nicotine 30 min later; or 1, 5, 10, 20, or 30 mg/kg of imatinib mesylate is prepared into a mixed injection with 0.15 mg/kg of nicotine and then administered. After 60 min, a spontaneous activity detecting apparatus was used to detect the spontaneous activities.

III. Experimental Results

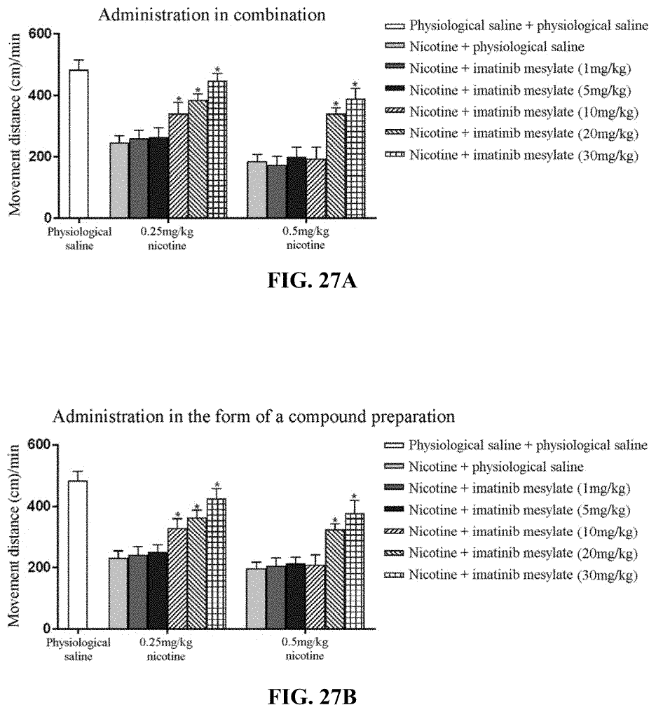

The results are shown in FIG. 27A. after the rats with nicotine addiction are given mecamylamine, the withdrawal symptoms are aroused, and the spontaneous activities decreases significantly. Regardless of administration in combination or in the form of a compound preparation, after imatinib mesylate is administered, 10, 20, and 30 mg/kg, but not 1 and 5 mg/kg, of imatinib mesylate can differently alleviate the withdrawal symptoms of addiction trained by 0.25 mg/kg of nicotine, and increase the activities. FIG. 27B shows that 20 and 30 mg/kg, but not 1, 5, and 10 mg/kg of imatinib mesylate can alleviate the withdrawal symptoms of addiction trained by 0.5 mg/kg of nicotine, and increase the activities. The results indicate that imatinib mesylate can alleviate the nicotine withdrawal symptoms when the dose ratio of imatinib mesylate to nicotine is 40:1 or greater.

### Example 23

Effect of imatinib mesylate on nicotine addiction in patients

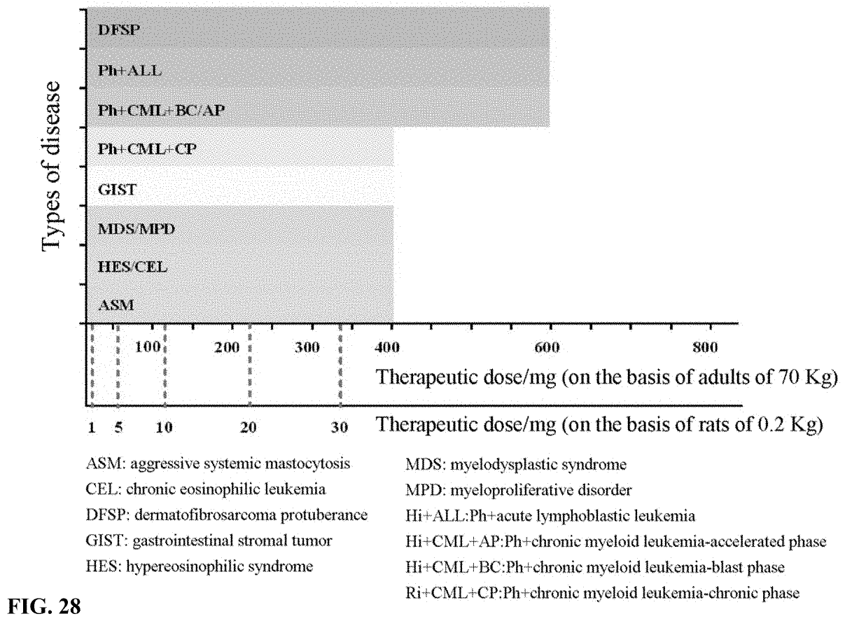

According to the above examples, imatinib mesylate inhibits the development of nicotine addiction and relapse in rats by inhibiting the phosphorylation activity of c-kit in nucleus accumbens. The effective dose is at 10-30 mg/kg, and the corresponding safe dose ranges are shown in FIG. 28. Imatinib mesylate is a clinically used drug, having few side effects when used at a daily dose of 400 mg or less. Based on the above efficacy and safety, an ICH-GCP clinical trial (registration batch number: CHICTR1800019507) was approved by the ethics committee to explore the effect of imatinib mesylate on nicotine addiction in patients.

I. Drug: imatinib mesylate (100 mg/tablet, available from Novartis).

Objective: To evaluate the efficacy of imatinib mesylate tablets in the treatment of patients with substance addiction.

Evaluation Indicators:

1) the changes in withdrawal symptoms of patients after taking the drug;

2) the changes in the patient's psychological craving after taking the drug;

3) vital signs (including heart rate, body temperature, respiration, blood pressure), and physical examination, etc.

II. Experimental Design

A randomized double-blind control design was used, including a screening period, a treatment period, and a 30-day follow-up period.

Criteria of Inclusion:

patients of voluntary abstinence, meeting the DSM-V-TR diagnostic criteria for substance dependence;

aged 18-60 (inclusive), having no other mental and physical diseases, male or female;

BMI: 19-35 kg/m² (inclusive);

opioid-dependent patients in 1-180 days after withdrawal, patents in 1-3 days after nicotine and alcohol withdrawal;

able to understand the procedures and methods of this study, willing to strictly follow the clinical trial protocol to complete this trial, and voluntarily sign the informed consent form.

Criteria of Exclusion:

hematological diseases;

history of allergy to alcohol or iodophor;

gastrointestinal diseases;

people who have received blood transfusion or blood donation;

pregnant or breastfeeding women, or men or women of childbearing potential who are unwilling to use contraception during the trial;

cardiovascular and cerebrovascular diseases and metabolic diseases, etc.;

people having active infection;

any other factors present in the patients that may affect the efficacy or safety evaluation of this study determined by the researchers.

III. Safety Evaluation

Any adverse events (especially adverse events such as urinary retention, systemic edema, and, increased granulocytes), vital signs, physical examination, electrocardiogram and other examination results are all used as indicators for safety evaluation.

IV Test Protocol

After a screening period of <1 day, the screened voluntary subjects with nicotine dependence entered a 3-day treatment period, and 10 patients in each group received drug treatment. After smoking a small amount of cigarettes in the morning, the patients were orally administered once a day, 3 tablets each time, for 3 consecutive days. After the treatment period, the patients were followed up by telephone calls once a day for 7 days, and once a week for 30 days. During the whole study period, the withdrawal symptoms and psychological craving were evaluated respectively before and after administration and during the follow-up period, and a relevant evaluation form was filled out according to the physical and mental state. During the study period, the patients were hospitalized for observation, could not be given other drug treatment than those specified in the protocol, and had to follow the doctor's treatment protocol.

V. Experimental Results

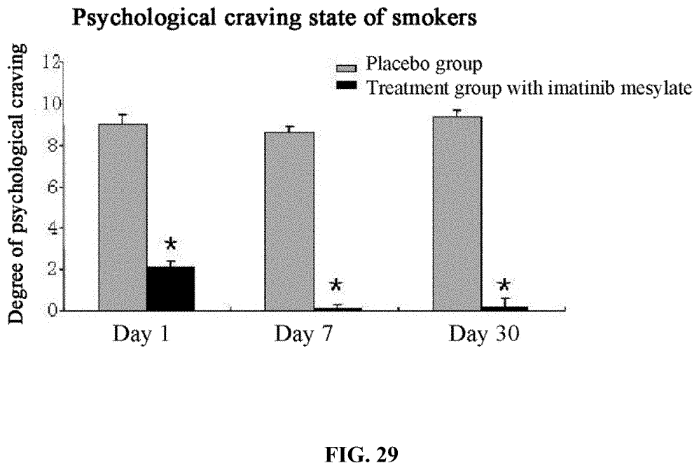

The results of the clinical study of smoking cessation in subjects show that after 3 days of administration and follow-up on days 1, 7, and 30, the nicotine addiction can be controlled by 80-90% or more, and can be completely quit; and the effect was very good, as shown in FIG. 29. The results have laid a solid foundation for drug efficacy and safety research and application of new drugs, and are expected to be applied to the clinical pharmaceutical market, to change the current situation of no drug treatment available.

Conclusions: imatinib or its derivative imatinib mesylate with nicotine or analogue thereof at a dose ratio of 40:1 or greater can control the development and relapse of addiction, and relieve the withdrawal symptoms after nicotine addiction, where the dose is in a safe range of clinical dose at present, as shown in FIG. 28. Moreover, compared with the relapse of nicotine addiction induced by environmental clue, imatinib mesylate has better preventive and therapeutic effect on relapse induced with a small dose of nicotine or an analogue thereof, where the ratio of dose of nicotine for induction to the dose for training nicotine addiction needs to be less than 3:10. In this way, the use of imatinib or a derivative thereof in combination with or in the form of a compound preparation with nicotine or an analogue thereof is feasible, effective, highly safe, and highly clinically controllable in the treatment of nicotine addiction, and in the control of relapse, making a substantial progress in nicotine addiction treatment and relapse control.

Examples 24 to 30 provide use of imatinib or a derivative thereof with an analgesic agent in analgesics.

The analgesic agent used in the following examples is morphine. Morphine is widely representative, and those skilled in the art can reproduce similar research results with other opioids having similar mechanism of action to morphine. Materials and reagents involved in the examples are commercially available, unless otherwise specified.

### Example 24

Effect of imatinib mesylate with morphine at a certain dose ratio on development of morphine addiction in rats and molecular mechanism

Test 24: Effect of imatinib mesylate with morphine at a certain dose ratio on development of morphine addiction in rats

I. Material

Drug: morphine (Qinghai Pharmaceutical), imatinib mesylate (Selleck Chemicals).

Test animals: SPF-grade male SD rats weighing 180-220 g, provided by Hunan SJA Laboratory Animal Co., Ltd, Animal Certificate No.: 43004700040706, and Production License No.: SCXK (Hunan) 2016-0002. Rat feed: purchased from the Laboratory Animal Center of Wuhan University.

Test instrument: Conditional place preference (CPP) test apparatus (developed by the Institute of Chinese Materia Medica, China Academy of Chinese Medical Sciences). The test was automatically controlled by a computer. The apparatus is a three-compartmented CPP box, inducing two side compartments and a middle compartment, where the three compartments are separated by removable partitions and are black internally and externally. The compartments A and B are located at two sides of the middle box and have the same size. 9 yellow light-emitting diodes are provided on a side wall of the compartment A to form a square array, and the bottom plate is formed of stainless steel strips. The bottom plate of the compartment B is formed of a stainless steel mesh. The data regarding time of rats spent in each compartment and the number of entries can be transmitted to the computer, and the behavioral data is automatically collected and recorded.

II. Experimental Method:

Baseline test: On day 1, the partitions were removed to open the passages between the three compartments, and the CPP program on the computer was started. The rats were transferred to the middle compartment, and allowed to move freely in the three compartments for 15 min. The time spent in each compartment was simultaneously recorded. According to the test results, some rats were excluded, and the remaining rats were divided into 6 groups (morphine+physiological saline group, morphine+imatinib mesylate group (1 mg/kg), morphine+imatinib mesylate group (5 mg/kg), morphine+imatinib mesylate group (10 mg/kg), morphine+imatinib mesylate group (20 mg/kg), and morphine+imatinib mesylate group (30 mg/kg)) each having 10 animals, and assigned to a drug-paired side or a nondrug-paired side.

CPP training: On days 2 to 9, the passages between the three compartment were closed. Imatinib mesylate was administered in combination with or in the form of a compound preparation with morphine. On days 2, 4, 6, and 8, the rats in each group were intraperitoneally injected with different doses of imatinib mesylate (1, 5, 10, 20, and 30 mg/kg, i.p.) or physiological saline (1 mL/kg, i.p.), and then subcutaneously injected with morphine (5, or 10 mg/kg, S.c.) 30 min later; or the rats in each group were subcutaneously injected with a mixed agent of imatinib mesylate (1, 5, 10, 20, 30 mg/kg, s.c.) or physiological saline (1 mL/kg, s.c.) with morphine (5, or 10 mg/kg, s.c.) at various dose ratios. Then the rats were transferred to the drug-paired side for 45 min. On days 3, 5, 7, and 9, the rats in morphine group were intraperitoneally injected with physiological saline (1 mL/kg, i.p.), and then subcutaneously injected with physiological saline (1 mL/kg, s.c.) 30 min later; or the rats in each group were subcutaneously injected with physiological saline (1 mL/kg, s.c.), and transferred to the nondrug-paired side for 45 min. The drug-paired side for each rat was constant. The rats in each group were returned to the rearing cage after the test.

Test of morphine-induced CPP: On day 10, CPP was tested. Similar to the baseline test, the passages between the three compartments were opened and the rats did not receive any injection. the CPP program on the computer was started. The rats were transferred to the middle compartment, and allowed to move freely in the compartments for 15 min. The time spent in each compartment was simultaneously recorded. CPP score is the difference between the time spent in the drug-paired side and the time spent in the nondrug-paired side. The CPP scores tested before and after training in the CPP test apparatus were compared to determine whether CPP is developed in the rats.

III. Experimental Results

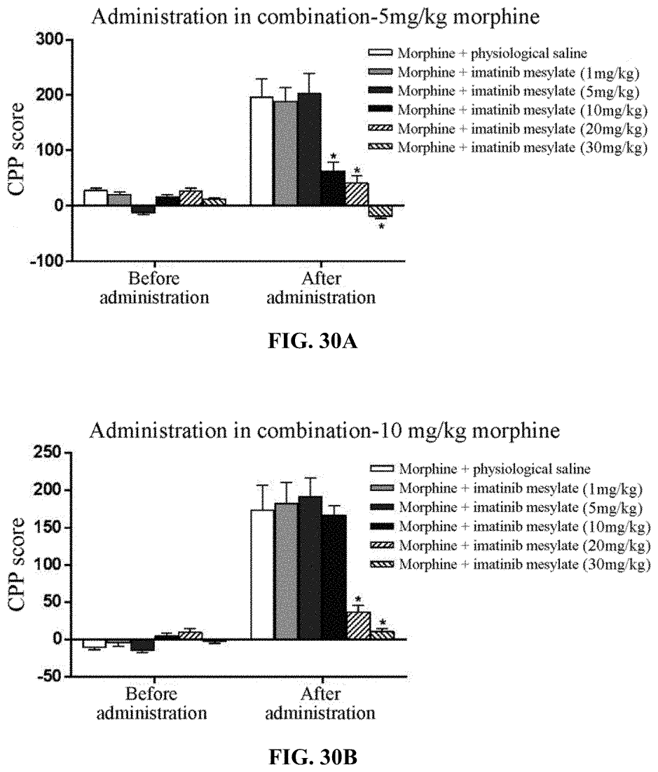

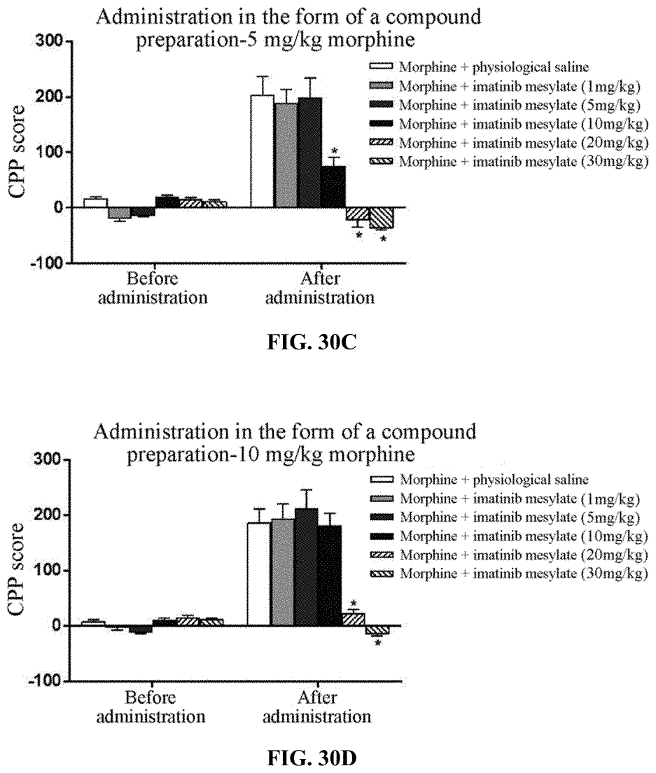

The results are shown in FIG. 30A. The CPP score in the groups receiving combined administration of imatinib mesylate+morphine has significant difference from that in the physiological saline+morphine group, and is dose-ratio dependent. Imatinib sulfonate intraperitoneally injected at a dose of 10, 20, and 30 mg/kg can inhibit the development of CPP in rats induced by 5 mg/kg of morphine; and 1, and 5 mg/kg of imatinib mesylate do not have this inhibitory effect. CPP still exists in rats not receiving treatment with imatinib mesylate. FIG. 30B shows that imatinib sulfonate intraperitoneally injected at a dose of 20, and 30 mg/kg can inhibit the development of CPP in rats trained by 10 mg/kg of morphine; and 1, 5, and 10 mg/kg of imatinib mesylate do not have this inhibitory effect. The results indicate that imatinib mesylate can inhibit the development of morphine addiction in rats when the dose ratio of imatinib mesylate to morphine is 2:1 or greater. Similarly, similar results are obtained for the administration of imatinib mesylate and nicotine in the form of a compound preparation in FIGS. 30C and 30D.

Test 25: New molecular mechanism of imatinib mesylate in preventing morphine addiction

It can be known from the results of test 24 that regardless of administration in combination or in the form of a compound preparation, imatinib mesylate (10-30 mg/kg) has different inhibitions on the development of morphine-induced CPP. In this test, immunohistochemistry, western-blot and multicolor immunofluorescence co-labeling were used to detect the changes of phosphorylation level of c-kit and its downstream activated target molecules in the brain region of drug reward, to determine the new molecular mechanism of morphine addiction and the mechanism of action of imatinib mesylate in controlling addiction.

I. Material

Drug: morphine (Qinghai Pharmaceutical), imatinib mesylate (Selleck Chemicals).

Test animals: SPF-grade male SD rats weighing 180-220 g, provided by the Laboratory Animal Center of China Three Gorges University. Animal Certificate No.: 42010200001637, Production License No.: SCXK (Hubei) 2016-0002. Rat feed: purchased from the Laboratory Animal Center of Wuhan University.

II. Experimental Method:

The animals were randomized to a physiological saline+physiological saline group, a physiological saline+imatinib mesylate group, a morphine+physiological saline group, and a morphine+imatinib mesylate group, each group having ten animals. The rats in each group were intraperitoneally administered with physiological saline (1 ml/kg, i.p.) or imatinib mesylate (30 mg/kg, i.p.), and then subcutaneously injected with morphine (10 mg/kg, s.c.) 30 min later. After 60 min, the changes of c-kit activity in the mesolimbic dopamine system, including VTA, nucleus accumbens, amygdala, hippocampus, and prefrontal cortex were observed, the changes in phosphorylation level of c-kit were observed by immunohistochemistry combined with western-blot, the distribution of activated cells was observed by immunofluorescence co-labeling, and the downstream activated target molecules were determined by multicolor immunofluorescence co-labeling, to determine the new molecular mechanism of morphine addiction and the mechanism of action of imatinib mesylate in controlling addiction.

III. Experimental Results

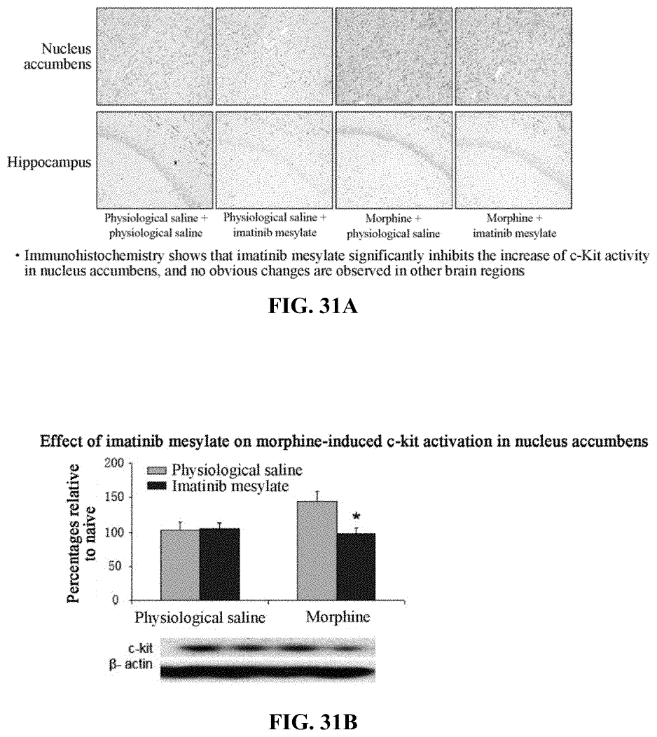

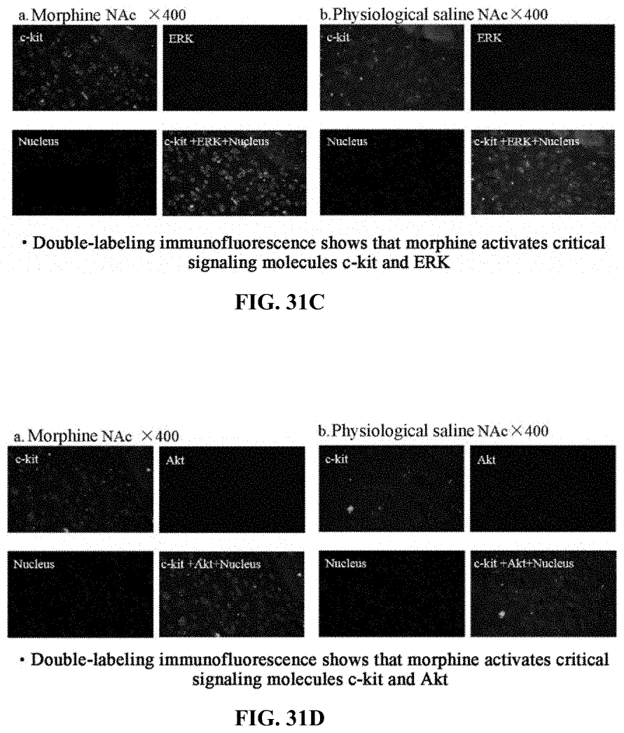

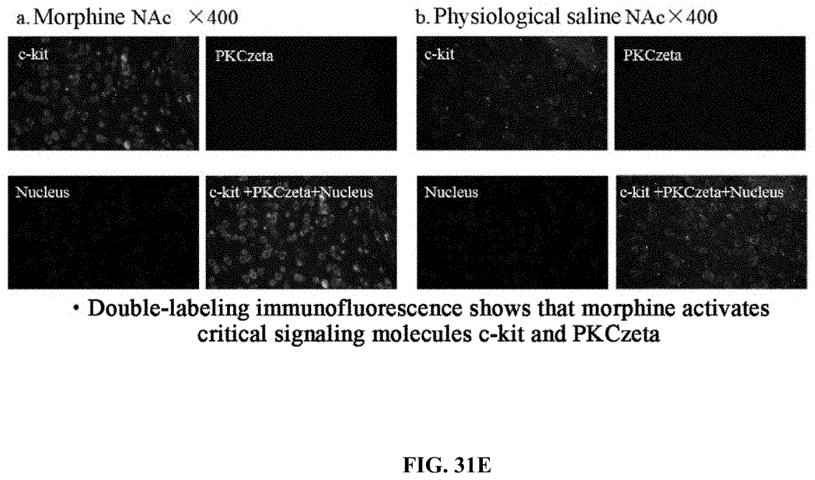

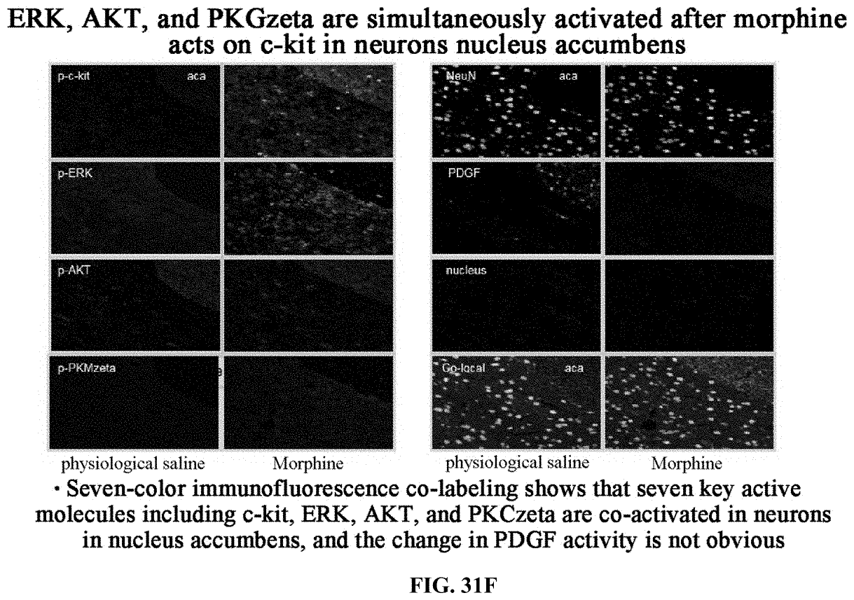

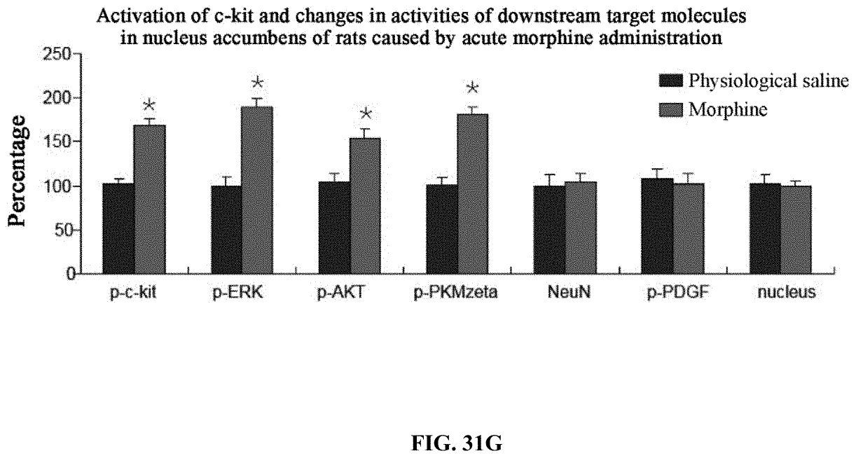

The results are shown in FIGS. 31A-31G. After acute morphine administration, the results of immunohistochemistry, western-blot and multi-color immunofluorescence co-labeling show that the c-kit receptor, and multiple downstream signaling molecules such as PKC, PI3K-AKT, ERK, and other regulatory kinases, protein expressions, gene expressions, and regulation and initiation of the process of morphine reward, memory and neuroplasticity in neurons in nucleus accumbens instead of in other brain regions are specifically activated. The results suggest that the c-kit receptor in nucleus accumbens is a brain region specifically activated by acute morphine administration. Imatinib mesylate inhibits multiple signaling molecules such as PKC, PI3K-AKT, ERK, and other regulatory kinases, protein expressions, gene expressions, and regulation and initiation of the process of morphine reward, memory and neuroplasticity by blocking the c-kit receptor, to achieve the effect of preventing morphine addiction.

### Example 25

Effect of imatinib mesylate with morphine at a certain dose ratio on development of addiction during morphine analgesia

According to Example 24, imatinib or its derivative imatinib mesylate inhibits the development of morphine addiction in rats by inhibiting the phosphorylation activity of c-kit in nucleus accumbens. Opioids such as morphine are commonly used analgesics in clinic. When imatinib mesylate is used with opioids at a dose ratio of 2:1 or greater, the side effect of morphine addiction can be prevented. In this example, imatinib mesylate and morphine were used as an analgesic agent for acute visceral pain caused by acetic acid in mice. An acetic acid-induced pain model and a CPP model in mice were established, to investigate the effect of imatinib mesylate with morphine at a certain dose ratio on the development of morphine addiction in mice with acute visceral pain.

I. Material

Drug: morphine (Qinghai Pharmaceutical), imatinib mesylate (Selleck Chemicals).

Test animals: SPF-grade male Kunming mice weighing 18-22 g, provided by the Laboratory Animal Center of China Three Gorges University. Animal Certificate No.: 42010200001574, Production License No.: SCXK (Hubei) 2017-0012. Rat feed: purchased from the Laboratory Animal Center of Wuhan University.

Test instrument: Conditional place preference (CPP) test apparatus (developed by the Institute of Chinese Materia Medica, China Academy of Chinese Medical Sciences). The test was automatically controlled by a computer. The apparatus is a three-compartmented CPP box, inducing two side compartments and a middle compartment, where the three compartments are separated by removable partitions and are black internally and externally. The compartments A and B are located at two sides of the middle box and have the same size. 9 yellow light-emitting diodes are provided on a side wall of the compartment A to form a square array, and the bottom plate is formed of stainless steel strips. The bottom plate of the compartment B is formed of a stainless steel mesh. The data regarding time of mice spent in each compartment and the number of entries can be transmitted to the computer, and the behavioral data is automatically collected and recorded.

II. Experimental Method:

Baseline test: The method was the same as that in Example 24.

CPP training: On days 2 to 9, the passages between the three compartment were closed. Imatinib mesylate was administered in the form of a compound preparation with morphine. On days 2, 4, 6, and 8, the mice in each group were intraperitoneally injected with 0.6% acetic solution (0.2 ml, i.p.), and then subcutaneously injected with a mixed agent of imatinib mesylate imatinib mesylate (1.5, 7.5, 15, 30, or 45 mg/kg, respectively corresponding to an equivalent dose in rats of 1, 5, 10, 20, and 30 mg/kg, s.c.) or physiological saline (1 mL/kg, s.c.) with morphine (10, or 15 mg/kg, s.c.) at various dose ratios. Then the mice were transferred to the drug-paired side for 45 min. On days 3, 5, 7, and 9, the mice in each group were intraperitoneally injected with 0.6% acetic solution (0.2 mL, i.p.), then subcutaneously injected with physiological saline (1 mL/kg, s.c.). transferred to the nondrug-paired side for 45 min. The drug-paired side for each mouse was constant. The mice in each group were returned to the rearing cage after the test.

Test of morphine-induced CPP: The method was the same as that in Example 24.

III. Experimental Results

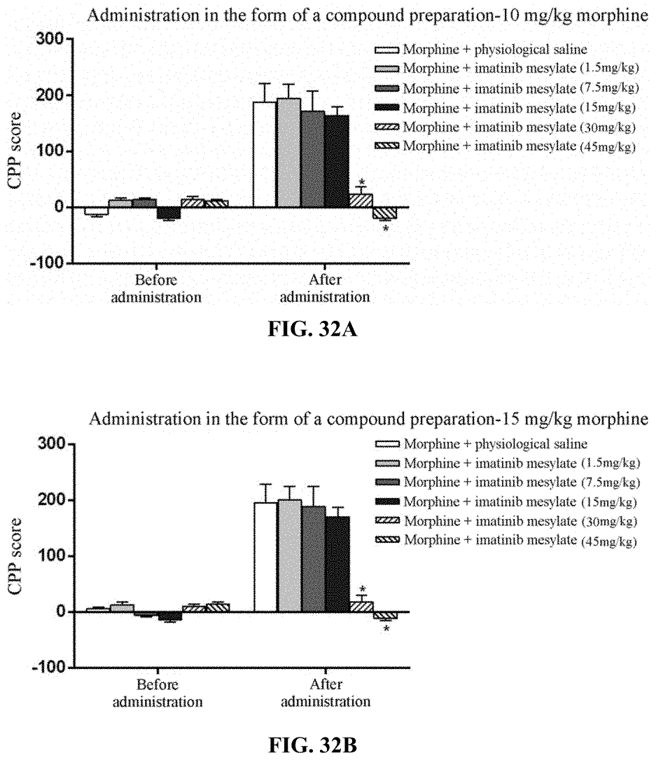

The results are shown in FIG. 32A. During morphine analgesia, the treatment group receiving administration of imatinib mesylate in the form of a compound preparation with morphine has significant difference from the morphine+physiological saline group, and is dose-ratio dependent. After the mice are subcutaneously injected with 30, or 45 mg/kg of imatinib mesylate, CPP cannot be developed in mice receiving 10 mg/kg of morphine; however, 1.5, 7.5, and 15 mg/kg of imatinib mesylate do not have this inhibitory effect. In mice not receiving imatinib mesylate, CPP still exists. FIG. 32B shows that after the mice are subcutaneously injected with 30, or 45 mg/kg of imatinib mesylate, CPP cannot be developed in mice receiving 15 mg/kg of morphine; however, 1.5, 7.5, and 15 mg/kg of imatinib mesylate do not have this inhibitory effect. The results indicate that imatinib mesylate with morphine can inhibit the development of addiction in rats during morphine analgesia in a dose ratio-dependent manner, where the dose ratio of imatinib mesylate to morphine is 2:1 or greater.

### Example 26

Effect of imatinib mesylate on morphine tolerance in rats

According to the results of Example 24 and Example 25, a composition of imatinib mesylate with morphine at a dose ratio of 2:1 or greater can significantly prevent the development of morphine addiction in rats or mice. In this example, imatinib mesylate and morphine were used as an analgesic agent for central analgesia in rats. A morphine tolerance and a hot-plate pain model in rats were established, to explore the effect of imatinib mesylate administered in combination with or in the form of a compound preparation with morphine on morphine tolerance.

I. Material

Drug: morphine (Qinghai Pharmaceutical), imatinib mesylate (Selleck Chemicals).

Test animals: SPF-grade female SD rats, weighing 180-220 g, provided by the Laboratory Animal Center of China Three Gorges University. Animal Certificate No.: 42010200001863, Production License No.: SCXK (Hubei) 2017-0012.

II. Experimental Method:

Determination of baseline pain threshold: The baseline pain threshold of rats was measured by the hot-plate method at 50±0.5° C. and expressed as the time when the rat licked the hind paw for the first time. To avoid the scalding of rats, the measurement time was less than 60 s, and rats having a time of greater than 30 s and less than 5 s were excluded. Two measurements were averaged. According to the baseline pain threshold, the rats were divided into 6 groups (morphine+physiological saline group, morphine+imatinib mesylate group (1 mg/kg), morphine+imatinib mesylate group (5 mg/kg), morphine+imatinib mesylate group (10 mg/kg), morphine+imatinib mesylate group (20 mg/kg), and morphine+imatinib mesylate group (30 mg/kg)) each having 10 animals.

The rats in each group were intraperitoneally injected with different doses of imatinib mesylate (1, 5, 10, 20, and 30 mg/kg, i.p.) or physiological saline (1 mL/kg, i.p.), and then subcutaneously injected with morphine (10 mg/kg, s.c.) 30 min later; or the rats in each group were subcutaneously injected with a mixed agent of imatinib mesylate (1, 5, 10, 20, or 30 mg/kg, s.c.) or physiological saline (1 mL/kg, s.c.) with morphine (10 mg/kg, s.c.) at various dose ratios. The pain threshold of the rats was measured after 30 min. The administration was continued for four consecutive days, and the pain threshold was measured daily after administration. On day 5, the rats in each group were given morphine subcutaneously (10 mg/kg, s.c.), and the heat pain threshold was determined.

III. Experimental Results

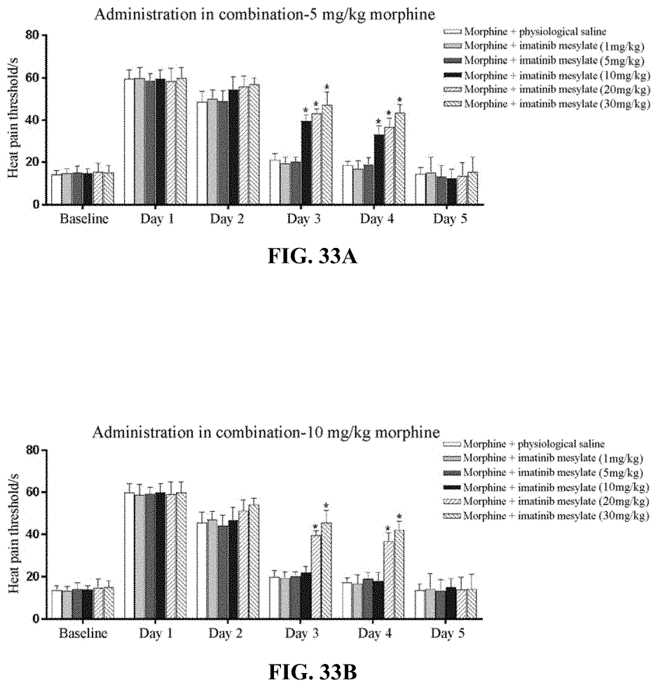

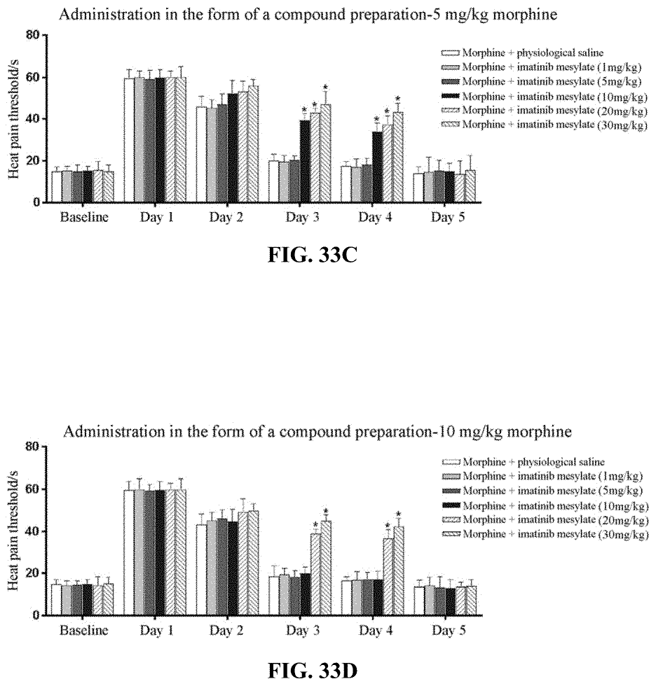

The results are shown in FIG. 33A. The heat pain threshold in the groups receiving combined administration of imatinib mesylate+morphine has significant difference from that in the physiological saline+morphine group, and is dose-ratio dependent. Imatinib sulfonate intraperitoneally injected at a dose of 20, and 30 mg/kg can significantly reduce the expression of morphine-induced tolerance in rats; however, 1, 5, and 10 mg/kg of imatinib mesylate do not have this inhibitory effect. In mice not receiving imatinib mesylate, the morphine tolerance still exists in rats. The results indicate that imatinib mesylate with morphine can inhibit the development of morphine tolerance in rats in a dose ratio-dependent manner, where the dose ratio of imatinib mesylate to morphine is 2:1 or greater. Similarly, similar results are obtained for the administration of imatinib mesylate and nicotine in the form of compound preparation in FIG. 33B.

### Example 27

Effect of imatinib mesylate with morphine at a certain dose ratio on central analgesic effect of morphine in rats

According to the results of Examples 24, 25 and 26, imatinib or its derivative imatinib mesylate can prevent the development of morphine addiction and the expression of morphine tolerance. Opioids such as morphine are commonly used analgesics in clinic. When imatinib mesylate is used with opioids at a dose ratio of 2:1 or greater, the side effect can be prevented. In this example, imatinib mesylate and morphine were used as an analgesic agent for central hot-plate pain in rats. A hot-plate pain model in rats was established, to investigate the effect of imatinib mesylate administered in combination with or in the form of a compound preparation with morphine on central analgesic effect of morphine in hot-plate induced pain in rats.

I. Material

Drug: morphine (Qinghai Pharmaceutical), imatinib mesylate (Selleck Chemicals).

Test animals: SPF-grade female SD rats, weighing 180-220 g, Provided by Hubei Laboratory Animal Research Center, Animal Certificate No.: 42000600025195, Production License No.: SCXK (Hubei) 2015-0018. Rat feed: purchased from the Laboratory Animal Center of Wuhan University.

II. Experimental Method:

Determination of baseline pain threshold: The method was the same as that in Example 26. According to the pain threshold time, the rats were randomly divided into 4 groups (imatinib mesylate+morphine group, imatinib mesylate+physiological saline group, physiological saline+morphine group, and physiological saline+physiological saline group), each having 10 animals.

The rats in each group were intraperitoneally injected with different doses of imatinib mesylate (1, 5, 10, 20, and 30 mg/kg, i.p.) or physiological saline (1 mL/kg, i.p.), and then subcutaneously injected with morphine (10 mg/kg, s.c.) or physiological saline (1 mL/kg, s.c.) 30 min later; or the four groups of rats were subcutaneously injected with imatinib mesylate (1, 5, 10, 20, or 30 mg/kg, s.c.) or physiological saline (1 mL/kg, s.c.) with morphine (10 mg/kg, s.c.) or physiological saline (1 mL/kg, s.c.) at various dose ratios. After 30 min, the rats were gently placed on a hot plate at 50±0.5° C., and the time when the rats licked the hind paw for the first time was recorded. If the rats did not lick the paw after 60 s, the rat were removed, and the pain threshold time was recorded as 60 s. The performance of rats before and after administration was observed, and the observations were carefully recorded.

III. Experimental Results

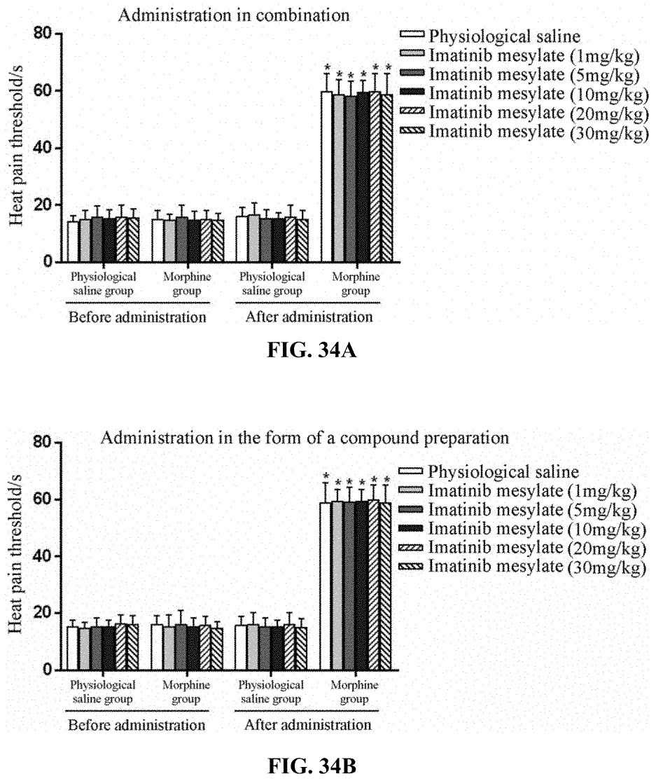

The results are shown in FIG. 34A. the heat pain threshold in rats in the groups receiving combined administration of imatinib mesylate+morphine has no obvious change compared with the morphine+physiological saline group; and HPT in rats in groups receiving imatinib mesylate+physiological saline also has no obvious change compared with the physiological saline+physiological saline group. The results show that in the hot-plate pain test, the combination of imatinib mesylate and morphine will not weaken the central analgesic effect of morphine regardless of the dose ratio. Similarly, similar results are obtained for the administration of imatinib mesylate and nicotine in the form of compound preparation in FIG. 34B.

### Example 28

Effect of imatinib mesylate with morphine at a certain dose ratio on analgesic effect of morphine in acute visceral pain of morphine in mice

According to the results of Example 27, imatinib mesylate has no obvious influence on the central analgesic effect of morphine in rats. In this example, imatinib mesylate and morphine were used as an analgesic agent for acute visceral pain in mice. An acetic acid-induced writhing pain model in mice was established, to investigate the effect of imatinib mesylate administered in combination with or in the form of a compound preparation with morphine on visceral analgesic effect of morphine in acetic acid-induced writhing in mice.

I. Material

Drug: morphine (Qinghai Pharmaceutical), imatinib mesylate (Selleck Chemicals).

Test animals: SPF-grade male Kunming mice weighing 18-22 g, provided by the Laboratory Animal Center of China Three Gorges University. Animal Certificate No.: 42010200001139, License No.: SOCK (Hubei) 2015-0012.

II. Experimental Method:

The mice were randomized into four groups, including a physiological saline+physiological saline group, a physiological saline+morphine group, an imatinib mesylate+physiological saline group, and an imatinib mesylate+morphine group, each having 10 animals. The mice in each group were intraperitoneally injected with different doses of imatinib mesylate (1.5, 7.5, 15, 30, and 45 mg/kg, respectively corresponding to an equivalent dose in rats of 1, 5, 10, 20, and 30 mg/kg, i.p.) or physiological saline (1 mL/kg, i.p.), and then subcutaneously injected with morphine (10 mg/kg, s.c.) or physiological saline (1 mL/kg, s.c.) 30 min later; or the rats in each group were subcutaneously injected with different doses of imatinib mesylate (1.5, 7.5, 15, 30, and 45 mg/kg, respectively corresponding to an equivalent dose in rats of 1, 5, 10, 20, and 30 mg/kg, i.p.) or physiological saline (1 mL/kg, i.p.). 30 minutes later, the mice were intraperitoneally administered with 0.2 mL of 0.6% acetic acid solution, the latency of writhing response and the writhing times appearing within 0-20 minutes were observed. The writhing response was that the abdomen of the mice was contracted and concave, the body and hind limbs were stretched, the buttocks were cocked and the mice crawled wiggly.

III. Experimental Results

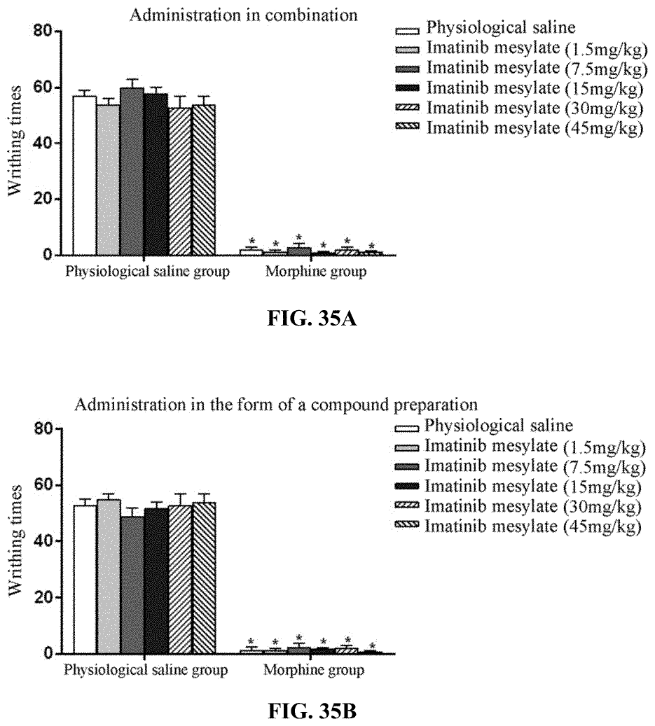

The results are shown in FIG. 35A. Compared with the morphine+physiological saline group, the writhing times in mice in the imatinib mesylate+morphine groups have no obvious changes. Compared with the physiological saline+physiological saline group, the writhing times in mice in the imatinib mesylate+physiological saline groups also have no obvious changes. The results show that in the acetic acid-induced writhing test, the combination of imatinib mesylate with morphine has no obvious influence on the analgesic effect of morphine in acute visceral pain of morphine in mice regardless of the dose ratio. Similarly, similar results are obtained for the administration of imatinib mesylate and nicotine in the form of compound preparation in FIG. 35B.

### Example 29

Effect of imatinib mesylate with morphine at a certain dose ratio on analgesic effect of morphine in acute chemical-induced inflammatory pain in rats

According to the results of Examples 27, and 28, imatinib mesylate has no obvious influence on the central analgesic effect of morphine in rats and the analgesic effect of morphine in acute visceral pain in mice. In this example, imatinib mesylate and morphine were used as an analgesic agent for acute inflammatory pain in rats. A test model of 5% formalin-induced pain in rats was established, to investigate the effect of imatinib mesylate administered in combination with or in the form of a compound preparation with morphine on the analgesic effect of morphine in 5% formalin-induced acute visceral pain in rats.

I. Material

Drug and reagent: morphine (Qinghai Pharmaceutical), imatinib mesylate (Selleck Chemicals).

Test animals: SPF-grade male SD rats weighing 180-220 g, provided by Hunan SJA Laboratory Animal Co., Ltd, Animal Certificate No.: 43004700041685, Production License No.: SOCK (Hunan) 2016-0002.

II. Experimental Method:

The mice were randomized into four groups, including a physiological saline+physiological saline group, a physiological saline+morphine group, an imatinib mesylate+physiological saline group, and an imatinib mesylate+morphine group, each having 10 animals. The rats in each group were intraperitoneally injected with different doses of imatinib mesylate (1, 5, 10, 20, or 30 mg/kg, i.p.) or physiological saline (1 mL/kg, i.p.), and then subcutaneously injected with morphine (10 mg/kg, s.c.) or physiological saline (1 mL/kg, s.c.) 30 min later; or the rats in each group were subcutaneously injected with a mixed agent of imatinib mesylate (1, 5, 10, 20, or 30 mg/kg, s.c.) or physiological saline (1 mL/kg, s.c.) with morphine (10 mg/kg, s.c.) or physiological saline (1 mL/kg, s.c.) at various dose ratios. After 30 min, 50 μL of 5% formalin solution was administered to the surface of the left hind paw of the rat, and the total time of licking to cause the left hind paw to become red and swollen was observed and recorded every five minutes for one hour. The formalin-induced pain test was divided into two stages, where 0-10 minutes was the first stage involving acute pain, which could be inhibited by central analgesic agents such as morphine; and 10-60 minutes was the second stage involving acute pathological pain caused by inflammatory reaction partially mediated by prostaglandin.

III. Experimental Results

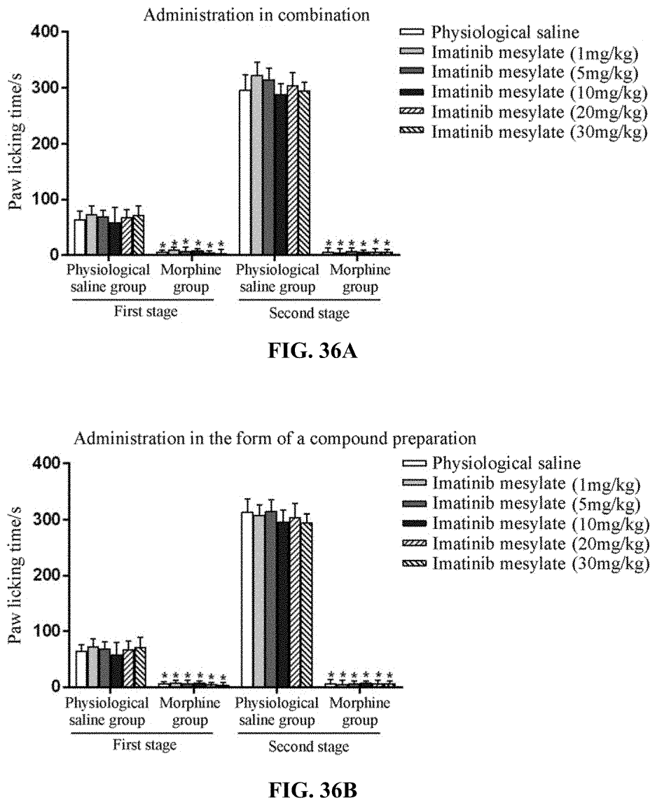

The results are shown in FIG. 36A. Compared with the morphine+physiological saline group, the hind paw licking time of rats in the imatinib mesylate+morphine groups have no obvious changes. Compared with the physiological saline+physiological saline group, the hind paw licking time of rats in the imatinib mesylate+physiological saline groups also have no obvious changes. The results show that in the formalin-induced pain test, the combination of imatinib mesylate with morphine has no influence on the analgesic effect of morphine regardless of the dose ratio. Similarly, similar results are obtained for the administration of imatinib mesylate and nicotine in the form of compound preparation in FIG. 36B.

### Example 30

Effect of imatinib mesylate with morphine at a certain dose ratio on analgesic effect of morphine in chronic inflammatory pain in rats

According to the results of Examples 27, 28 and Example 29, imatinib mesylate has no obvious influence on the analgesic effect of morphine in acute pain in rats and mice. In this example, imatinib mesylate and morphine were used as an analgesic agent for chronic inflammatory pain in rats. A rat model of chronic plantar pain induced by complete Freund's adjuvant (CFA) was established, to investigate the effect of imatinib mesylate administered in combination with or in the form of a compound preparation with morphine on the CFA-induced chronic inflammatory pain in rats.

I. Material

Drug: morphine (Qinghai Pharmaceutical), imatinib mesylate (Selleck Chemicals).

Test animals: SPF-grade female SD rats, weighing 180-220 g, provided by the Laboratory Animal Center of China Three Gorges University. Animal Certificate No.: 42010200001281, Production License No.: SOCK (Hubei) 2017-0012.

II. Experimental Method:

Determination of baseline pain threshold: The method was the same as that in Example 26.

Establishment of chronic inflammatory pain model: After determining the baseline pain threshold, rats with a normal threshold were selected, and randomly assigned to a CFA group (40) and an NS group (40, physiological saline). Establishment of CFA-induced chronic inflammatory plantar pain model: The rats in the CFA group were subcutaneously intraplantarly injected with 125 μL of CFA by a 1 mL syringe at the left hind paw, and the needle hole was pressed and massaged for a few minutes after injection to promote the drug diffusion. The rats in the NS group were injected with physiological saline of the same volume through the same route, to develop a control model. After the model establishment, the rats were weighed and the changes of the left paws of the rats (such as redness, swelling, presence of exudate or infection at the plantar injection site) were observed every day. the circumference crossing the highest part of the left paw was measured, and the circumference at the same part of the right paw was also measured for comparison.

Through days after the successful model establishment, the rats were administered. The pain threshold of the rats was measured on a hot plate before administration. Then, according to the circumference crossing the highest part of the left paw, the rats in the CFA group were randomly divided into the following four groups: a physiological saline+physiological saline group, a physiological saline+morphine group, an imatinib mesylate+physiological saline group, and an imatinib mesylate+morphine group, each group having 10 animals. The rats in the NS group were also randomly divided into the following four groups: a physiological saline+physiological saline group, a physiological saline+morphine group, an imatinib mesylate+physiological saline group, and an imatinib mesylate+morphine group, each having 10 animals. The rats in each group were intraperitoneally injected with different doses of imatinib mesylate (1, 5, 10, 20, or 30 mg/kg, i.p.) or physiological saline (1 mL/kg, i.p.), and then subcutaneously injected with morphine (10 mg/kg, s.c.) or physiological saline (1 mL/kg, s.c.) 30 min later; or the rats in each group were subcutaneously injected with a mixed agent of imatinib mesylate (1, 5, 10, 20, or 30 mg/kg, s.c.) or physiological saline (1 mL/kg, s.c.) with morphine (10 mg/kg, s.c.) or physiological saline (1 mL/kg, s.c.) at various dose ratios. The pain threshold of the rats was measured after 30 min. The administration was continued for three consecutive days, and the heat pain threshold was measured daily after administration. The duration of the entire experiment was one week.

III. Experimental Results

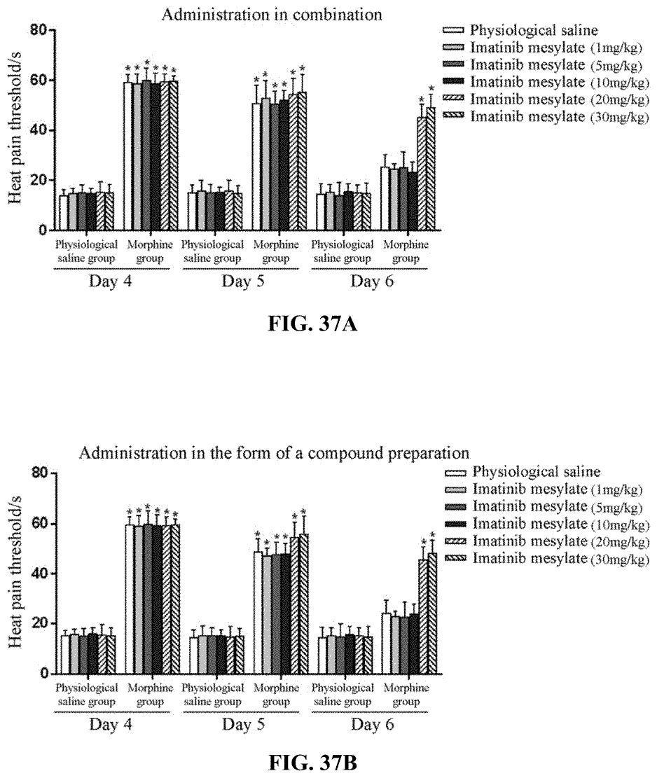

The results are shown in FIG. 37A. On days 4 and 5 after the model establishment, the heat pain threshold in rats in the groups receiving combined administration of imatinib mesylate+morphine has no obvious change compared with the morphine+physiological saline group; and on day 6 after the model establishment morphine tolerance is developed in rats. 20, and 30 mg/kg of imatinib mesylate can significantly prevent the development of morphine tolerance, and increase the heat pain threshold; however, 1, 5, and 10 mg/kg of imatinib mesylate have no this effect. The results show that in the test of chronic inflammatory plantar pain induced by Freund's complete adjuvant, imatinib mesylate has no influence on the analgesic effect of morphine, and can prevent the expression of morphine tolerance at a dose ratio to morphine of 2:1 or greater after the development of morphine tolerance. Similarly, similar results are obtained for the administration of imatinib mesylate and nicotine in the form of compound preparation in FIG. 37B.

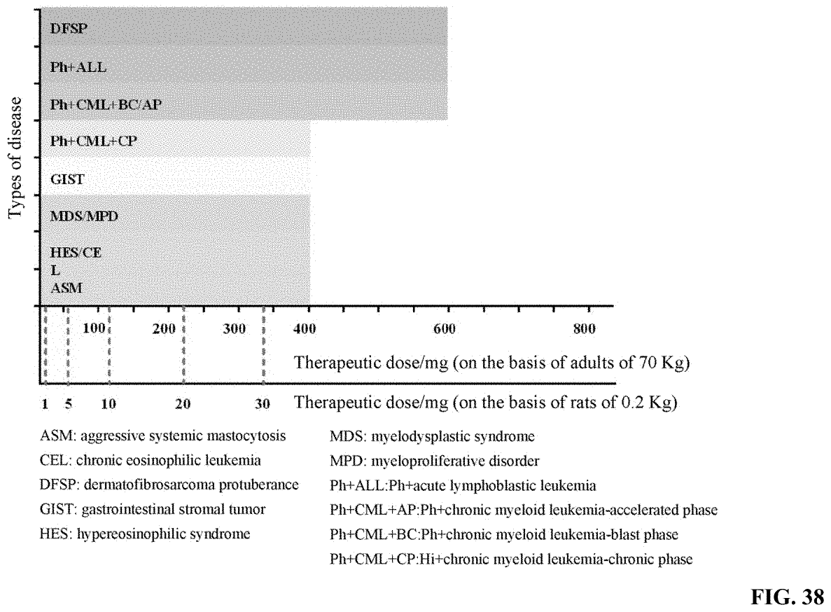

Conclusions: Imatinib or its derivative imatinib mesylate with morphine at a dose ratio of 2:1 or greater can control the development of addiction, and the expression of tolerance, where the dose is in a safe range of clinical dose at present, as shown in FIG. 38. Moreover, imatinib mesylate can used with opioid analgesics such as morphine in acute and chronic pains induced by hot plate, acetic acid, formalin and Freund's complete adjuvant, without affecting the analgesic effect of morphine. Therefore, imatinib or a derivative thereof can be used with opioid analgesics such as morphine at a certain dose ratio to prevent the development of addiction and tolerance side effects during pain treatment with opioids. With the formulation for preventing side effects, the two at a dose ratio of 2:1 or greater are applicable in the treatment of various types of pain, thus widening the scope of indications to which opioid analgesics are applicable.

Preferred embodiments of the disclosure have been described above; however, the disclosure is not limited thereto. Any other changes, modifications, alternatives, combinations, simplifications made without departing from the spirit and principle of the disclosure are all equivalent replacements, and embraced in the protection scope of the disclosure.

What is claimed is:

1. A method for prevention and treatment of addictions and control of relapse comprising administering a patient in need thereof a composition comprising imatinib or a derivative thereof, and an addictive substance or an analgesic agent; wherein the addictive substance comprises: (1) narcotic drugs, (2) psychotropic drugs, (3) ethanol, tobacco, volatile organic solvents, and other substances leading to addictions in organisms, and analogues thereof; and (4) nicotine or an analogue thereof.

2. The method of claim 1, wherein the composition comprises imatinib or a derivative thereof and the addictive substance, and is used for prevention and treatment of addictions, and control of relapse.

3. A method for prevention and treatment of addictions and control of relapse comprising administering a patient in need thereof a composition comprising imatinib or a derivative thereof, and an addictive food; and the addictive food comprising high-fat food, sweets, chocolate and other delicious foods.

4. The method of claim 3, wherein imatinib or a derivative thereof is in combination with or in the form of a compound preparation with the addictive food.

5. A method for prevention and treatment of addictive behaviors and control of relapse comprising administering a patient in need thereof imatinib or a derivative thereof; wherein the addictive behaviors comprise gambling addiction, Internet addiction, and other behaviors leading to addictions in organisms.

6. The method of claim 5, wherein the imatinib or a derivative thereof is used after induction with an addictive behavior clue.

7. The method of claim 2, wherein imatinib or a derivative thereof is used at a dose of 100-400 mg/day.

8. The method of claim 2, wherein when the addictive substance is morphine or cocaine, a dose ratio of imatinib or a derivative thereof to the addictive substance is 2:1 or greater; and when the addictive substance is ethanol, a dose ratio of imatinib or a derivative thereof to the addictive substance is 1:50 or greater.

9. The method of claim 2, wherein a ratio of a dose of the addictive substance in the prevention and treatment of addiction and control of relapse to a dose for training addiction is 1:3 or less.

10. The method of claim 2, wherein the composition is prepared into one of the following dosage forms: an injection, an infusion, a pill, a tablet, a powder, granules, a capsule, a powder, an oral liquid, a sustained-release preparation, a tincture, a suppository, and a patch.

11. The method of claim 2, wherein the addictive substance is nicotine or an analogue thereof.

12. The method of claim 11, wherein the composition comprises imatinib or a derivative thereof and nicotine or an analogue thereof, and is used for prevention and treatment of nicotine addiction, and for control of relapse.

13. The method of claim 11, wherein upon arousal of nicotine addiction memory, the dose used needs to be at a ratio to the dose for developing addiction of 3:10 or less based on the active ingredient, then imatinib or a derivative thereof is effective in the prevention and treatment of nicotine addiction and in the control of relapse.

14. The method of claim 1, wherein the composition comprises imatinib or a derivative thereof and an analgesic agent, and is used for treatment of pains.

15. The method of claim 14, wherein:
(1) the composition comprising imatinib or a derivative thereof and an analgesic agent is used for treatment of pains; and
(2) the composition comprising imatinib or a derivative thereof and an analgesic agent is used for preventing tolerance and addiction side effects of the analgesic drug.

16. The method of claim 15, wherein a ratio of imatinib or a derivative thereof to the analgesic agent in the composition is 2:1 or greater based on the active ingredients; and
clinically, and a dose of imatinib or a derivative thereof is 100-400 mg/day.

17. The method of claim 15, wherein the analgesic agent comprises addictive opioids that act on a central analgesia system to produce analgesic effects, comprising morphine, codeine, pethidine, fentanyl, methadone, oxycodone, hydromorphone, nalbuphine, and marijuana, and various non-opioid addictive compounds or their salts.

18. The method of claim 15, wherein the pains refer to the indications and other various types of acute and chronic pains that are suitably treated by the administration of the analgesic agent alone.

19. The method of claim 15, wherein in the composition comprising imatinib or a derivative thereof and an analgesic agent, imatinib or a derivative thereof and the analgesic agent are administered simultaneously, separately or sequentially.

20. The method of claim 15, wherein the composition is prepared into one of the following dosage forms: an injection, an infusion, a subcutaneous implant, a pill, a tablet, a powder, granules, a capsule, a powder, an oral liquid, a sustained-release preparation, a tincture, a suppository, and a patch.
